SEQUENCE LISTING (421 sequences)

SEQ ID NO: 1 - DNA, 314 bp, Homo sapiens
gatgttctcagaattgtctacatattaattagaaacaagactaaagaaatgtcaatcctgaggacaacagtgaacccctgtaattctaagtagcagtttgtctctgagacttatcagacacagggttttcactgccaaaggattacataaatattcagaacataaaggattttagccatcttgagggtgcttactttgactgctaaaaactgaaaagtgattcaatactgcttaagtgcatggcttataatgtaattattgaatcaagttatgttctaagaaattttttatactaatcatttttcaagaggcat

SEQ ID NO: 2 - DNA, 339 bp, Homo sapiens
tggtcgtgccaaaaagggccgcggccacgtgcagcctattcgctgcactaactgtgcccgatgcgtgcccaaggacaaggccattaagaaattcgtcattcgaaacatagtggaggccgcagcagtcagggacatttctgaagcgagcgtcttcgatgcctatgtgcttcccaagctgtatgtgaagctacattactgtgtgagttgtgcaattcacagcaaagtagtcaggaatcgatctcgtgaagcccgcaaggaccgaacacccccaccccgatttagacctgcgggtgctgccccagctcccccaccaaagcccatgtaaggagctgagttctt

SEQ ID NO: 3 - DNA, 407 bp, Homo sapiens
tttctttcttgttcaattcatctagattataaccttaatgtgacacctgagacctttagacagttgaccctgaattaaatagtcacatggtaacaattatgcactgtgtaattttagtaatgtataacatgcaatgatgcactttaactgaagatagagactatgttagaaaattgaactaatttaattatttgattgttttaatcctaaagcataagttagtcttttcctgattcttaaaggtcatacttgaaatcctgccaattttccccaaagggaatatggaattttttttgactttcttttgagcaataaaataattgtcttgccattacttagtatatgtagacttcatcccaattgtcaaacatcctaggtaagtggttgacatttcttacagcaattac

SEQ ID NO: 4 - DNA, 470 bp, Homo sapiens
gtgcagtgctacaagtaaatcgacttaaatcagcccagtgtggatcttcaatgatatatcattgtaagcataagctttagaagtaaagcatttgcgtttacagtgcatcagatacattttatatttcttaaaatagaaatattatgattgcataaatctgaaaatgaattatgttatttgctctaatacaaaaattctaaatcaattattgaaataggatgcacacaattactaaagtacagacatcctagcatttgtgtcgggctcattttgctcaacatggtatttgtggttttcagcctttctaaaagttgcatgttatgtgagtcagcttataggaagtaccaagaacagtcaaacccatggagacagaaagtagaatagtggttgccaatgtctcagggaggttgaaataggagatgaccactaattgatagaacgtttctttgtgtcgtgatgaaaactttcta

SEQ ID NO: 5 - DNA, 507 bp, Homo sapiens
  modified_base (65)..(65): a, c, t, or g
  modified_base (67)..(67): a, c, t, or g
  modified_base (70)..(70): a, c, t, or g
  modified_base (74)..(75): a, c, t, or g
  modified_base (81)..(81): a, c, t, or g
  modified_base (87)..(87): a, c, t, or g
  modified_base (100)..(100): a, c, t, or g
  modified_base (110)..(110): a, c, t, or g
  modified_base (114)..(114): a, c, t, or g
  modified_base (116)..(116): a, c, t, or g
  modified_base (127)..(127): a, c, t, or g
  modified_base (174)..(174): a, c, t, or g
atctgttcatccattgatgggttttgagtttacaccttgtggctattgtgaataactgctatgancnctnatgnncagatnatgttngcatttccgcttncagttctttngggngnatcttagaagnggaattgctgggtcataaaataattctgtgtttaatattttgagganccgcctgactgtcttcaacagtagctatactatttttgtttccaccatcaatgtgtaagggttccattttcttcatgtccttgccaacactgtcttttttaataatagtcatcctaatgggtataaagtggtatcttcttatgcatctcactaatgaaaacatttttttcatattttgcattagtttacatttgaaagtattgctttaaatttgtgaactatatatgagcaatgtagagaaatatttcgctttaagcaatggttgactggccaggtacaatgactcacacctgtaattccagcactttgggatgccgagttgagaggatcact

SEQ ID NO: 6 - DNA, 389 bp, Homo sapiens
  modified_base (125)..(125): a, c, t, or g
  modified_base (154)..(154): a, c, t, or g
  modified_base (242)..(242): a, c, t, or g
gtacatcagatacaacactcttctcatcttttttgtcattatttttcaatgttgttttaatctttccactgttgtttcatgtcatgcactttgttaatattctttaagttattcttagtttttancaatgatacttaagaaagacttaataggnctatagaataggtactccctattccccaaccctgggaacacaatataacatattagttcatttgtatttaagatctatcttgtttacncctcagtttagttattgctcttacctttcaaacagttaatattttgtatctagttctgtgaaagcaaaaagcaactttattctttcttacaattccccttttacttaatcggaacatttctttcctgtttgttcattagtcactctc

SEQ ID NO: 7 - DNA, 443 bp, Homo sapiens
  modified_base (308)..(308): a, c, t, or g
  modified_base (312)..(318): a, c, t, or g
  modified_base (320)..(327): a, c, t, or g
  modified_base (329)..(329): a, c, t, or g
  modified_base (331)..(333): a, c, t, or g
agattatggcctattgaacaaaatgtgaaactgggagtccacaaagacataaatggaaagatgaagtacctcctgacagagcaagaatggcactgaacaatgttatatggacaatggctattttattcaagactattacaatagggaaaagagactaggactcagtctgaactgaaatctgtcaaaacaaaggggggtggggctttaagagtgaaggtgaggaggagatcacacaccgtatgtgtttgctaactggctttacccaaaagaaaattaaactttctttgatctgtacaagttgatcaaanaaannnnnnnannnnnnnnanannnaactaaaacagggtagacaggccaggggaaaaaggcactcagggcacacagcattgcttcaaaatataattctctacaaacctagttgctaaaactacctgttgtaacct

SEQ ID NO: 8 - DNA, 500 bp, Homo sapiens
  modified_base (299)..(299): a, c, t, or g
  modified_base (456)..(456): a, c, t, or g
ttgtgtgtaatttcatggtggcctagtgttgtggtgcttctggtaatggtaatagaagctcaactatttttttgtggatttcagtttttatcatcagaagtcctagacagtgacatttcttaatggtgggagtccagctcatgcatttctgattatacaaaacagtttgcagtaggttatttgtcatttcagttttttactgaaatttgagctaaacatttttacatgtaaatacttgtatttaccaaagatttaaatcagttgattaattaattaactcaaatactgtgaactatctntaaaacactagaaaaaagaaatgttagtatctcaattacaccaactgtgcaaatgaactttgataaaatagaaataatctacattggcctttgtgaaatctggggaagagctttaggattctagtagatggatactgaatactcaggcccacttaanttattaatgtatacattgtgtttttgtctttatgctatgtacag

SEQ ID NO: 9 - DNA, 570 bp, Homo sapiens
  modified_base (159)..(159): a, c, t, or g
  modified_base (212)..(212): a, c, t, or g
ccatgtggatttgagggcagcagtccctggcctcaccctagccagcctgggtggctccctagccccaagaggccaggaagggctggaaggcagggcctgcaggtgctccccgccctgagacccaggccccaaatcagcaataatgaacaaacccttggncccagcctgggctggtgacctgggcaccagagaccttgcatccctcctcatcntaggaggcccctaggggtgccccatctcagtgtcccctgaactctttatttgcctaatttatatatatatatatgagatatataaatatatataaaatagctattttgcttaaatttctacagtatgtaaaagtgaaaaaatgatgaagacgggtgcacctgtctgagtttggccctcatgtgagctgtgcccttccctctcctcatgcccccttccagcggcttctgccaaccatggggggctggaccaccatggccactgacccagcccctcagaatcccacactccaatcctttccatttcagtttagtcctaaaagttcatcacagggtctttctttctactccaggactggtt

SEQ ID NO: 10 - DNA, 396 bp, Homo sapiens
tccctaacctacacccatagattaccaaggtttcagtgtactagttttgaatctgttctgaatggagtttttataccctcaatttctggcctttggctattttagcatttcaaagtgacttctatgaagctttttttttaatgtgaaattttcagaatgttgtttttttcatgtagatactccaggaagagttaagcactgctttcagttttaatatccaccttgaggggtcgctgcttgagggctcttatcccaggggactttttaattcggatgttacttaatgtggcttctctaatgtagtttctttgattaccgactacacaattatgtaccatcacagtattagtggaaaagtaccatgtgatttaattctccattcctccaatgtaactc

SEQ ID NO: 11 - protein, 297 aa, Artificial Sequence
  Description of Artificial Sequence: Synthetic polypeptide
  Isoform A precursor
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGEPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 12 - protein, 248 aa, Artificial Sequence
  Description of Artificial Sequence: Synthetic polypeptide
  Isoform B
MQRARPTLWAAALTLLVLLRGPPVARAGASSAGLGPVVRCEPCDARALAQCAPPPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 13 - skipped

SEQ ID NO: 14 - protein, 291 aa, Artificial Sequence
  Description of Artificial Sequence: Synthetic polypeptide
  Binding protein 3
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 15 - protein, 710 aa, Artificial Sequence
  Description of Artificial Sequence: Synthetic polypeptide
  Hepatocyte growth factor like protein precursor
MGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKHRGRVRESEMCTEGLLAPVGACEGDYGGPLACFTHNCWVLEGIIIPNRVCARSRWPAVFTRVSVFVDWIHKVMRLG

SEQ ID NO: 16 - protein, 565 aa, Artificial Sequence
  Description of Artificial Sequence: Synthetic polypeptide
  KDA protein
MTSRCSGAQSYLLHAVVPGPWQEDVADAEECAGRCGPLMDCWAFHYNVSSHGCQLLPWTQHSPHSRLWHSGRCDLFQEKDYIRTCIMNNGVGYRGTMATTVGGLSCQAWSHKFPNDHKYMPTLRNGLEENFCRNPDGDPGGPWCHTTDPAVRFQSCSIKSCRVAACVWCNGEEYRGAVDRTESGRECQRWDLQHPHQHPFEPGKFLDQGLDDNYCRNPDGSERPWCYTTDPQIEREFCDLPRCGSEAQPRQEATSVSCFRGKGEGYRGTANTTTGVPCQRWDAQIPHQHRFTPEKYACKDLRENFCWNLDGSEAPWCFTLRPGTRVGFCYQIRRCTDDVRPQDCYHGAGEQYRGTVSKTRKGVQCQRWSAETPHKLQFTFTSEPHAQLEENFCQPDGDSHGPWCYTMDPRTPFDYCALRRCADDQPPSILDPPNQVQFEKCGKRVDRLDQRRSKLRVAGGHPGNSPWTVSLRNRQGQHFCAGSLVKEQWILTARQCFSSCHMPLTGYEVWLGTLFQNPQHGEPGLQRVPVAKMLCGPSGSQLVLLKLERSVTLNQRVALICLPPE

SEQ ID NO: 17 - protein, 243 aa, Artificial Sequence
  Description of Artificial Sequence: Synthetic polypeptide
  Isoform short of complement factor H-related protein 2
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTSSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 18-20 - skipped (3 entries)

SEQ ID NO: 21 - protein, 270 aa, Artificial Sequence
  Description of Artificial Sequence: Synthetic polypeptide
  Isoform long of complement factor H-related protein 2
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 22 - protein, 458 aa, Artificial Sequence
  Description of Artificial Sequence: Synthetic polypeptide
  Carboxypeptidase N Catalytic chain
MSDLLSVFLHLLLLFKLVAPVTFRHHRYDDLVRTLYKVQNECPGITRVYSIGRSVEGRHLYVLEFSDHPGIHEPLEPEVKYVGNMHGNEALGRELMLQLSEFLCEEFRNRNQRIVQLIQDTRIHILPSMNPDGYEVAAAQGPNKPGYLVGRNNANGVDLNRNFPDLNTYIYYNEKYGGPNHHLPLPDNWKSQVEPETRAVIRWMHSFNFVLSANLHGGAVVANYPYDKSFEHRVRGVRRTASTPTPDDKLFQKLAKVYSYAHGWMFQGWNCGDYFPDGITNGASWYSLSKGMQDFNYLHTNCFEITLELSCDKFPPEEELQREWLGNREALIQFLEQVHQGIKGMVLDENYNNLANAVISVSGINHDVTSGDHGDYFRLLLPGIYTVSATAPGYDPETVTVTVGPAEPTLVNFHLKRSIPQVSPVRRAPSRRHGVRAKVQPQARKKEMEMRQLQRGPA

SEQ ID NO: 23 - protein, 784 aa, Artificial Sequence
  Description of Artificial Sequence: Synthetic polypeptide
  Cadherin-5
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 24 - skipped

SEQ ID NO: 25 - protein, 4563 aa, Artificial Sequence
  Description of Artificial Sequence: Synthetic polypeptide
  Apolipoprotein B-100
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 26 - skipped

SEQ ID NO: 27 - protein, 147 aa, Artificial Sequence
  Description of Artificial Sequence: Synthetic polypeptide
  Hemoglobin subunit delta
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENFRLLGNVLVCVLARNFGKEFTPQMQAAYQKVVAGVANALAHKYH

SEQ ID NO: 28 - protein, 147 aa, Artificial Sequence
  Description of Artificial Sequence: Synthetic polypeptide
  Hemoglobin subunit Beta
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 29-30 - skipped (2 entries)

SEQ ID NO: 31 - protein, 231 aa, Artificial Sequence
  Description of Artificial Sequence: Synthetic polypeptide
  Complement component 1, Q subcomponent, B chain
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNS

SEQ ID NO: 32 - protein, 253 aa, Artificial Sequence
  Description of Artificial Sequence: Synthetic polypeptide
  Complement component 1, Q subcomponent, B chain precursor
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 33 - protein, 474 aa, Artificial Sequence [sequence omitted]

SEQ ID NO: 34 - protein, 474 aa, Artificial Sequence
  Description of Artificial Sequence: Synthetic polypeptide
  Vitamin D-binding protein precursor
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 35 - protein, 559 aa, Artificial Sequence
  Description of Artificial Sequence: Synthetic polypeptide
  Complement component C9
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 36 - protein, 567 aa, Artificial Sequence [sequence omitted]

SEQ ID NO: 37 - DNA, 482 bp, Homo sapiens
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagcagtgacagtgcccagggctctaatgtgtctctcacggcttgtaaattgtgaaacagctgccctgtgtgggactgagt

SEQ ID NO: 38 - DNA, 465 bp, Homo sapiens
  modified_base (368)..(368): a, c, t, or g
cacagacccagacccagagctgtgtgcctcccactgcaggagccagacaagcccctgagtctccatctaccatccctgtcccttcacacccgtcctctccccttcctgtccctctgccttccaggccacagaactccacgctccgccctggccctgcagcccccattgccctggtgcctgtgttctgtggactcctcgtagccaagagcctggtgctgtcagccctgctcgtctggtgggttttaaggaatcggcacatgcagcatcaagggaggtctctgctgcacccagctcagcccaggccccaggcccatagacacttcccactgagccacagggcaccaggggggacatatggtggaaaaccntgatggagctcaggagcctggatacccaaaaagccacctgccaccttcaacaggtcacggaccttccctggacctcagtttcctcacctgtagagag

SEQ ID NO: 39 - DNA, 342 bp, Homo sapiens
gacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtgcatgtgtagcctgtgcatgtatgtcttggacactgtacaaggtacccctttctggctctcccatttcctgagaccaccagagagaggggagaagcctgggattgacagaagcttctgcccacctacttttctttcctcagatcatccagaagttcctgaagggccaggactttatctaatacctctgtgtgctc

SEQ ID NO: 40 - DNA, 442 bp, Homo sapiens
gagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctgg

SEQ ID NO: 41 - DNA, 588 bp, Homo sapiens
  modified_base (47)..(47): a, c, t, or g
  modified_base (63)..(63): a, c, t, or g
  modified_base (260)..(260): a, c, t, or g
aagggccggaggtcatggccttcatcgagcagggcaagcggatggantgcccaccagagtgtnccacccgaactgtacgcactcatgagtgactgctggatctacaagtgggaggatcgccccgacttcctgaccgtggagcagcgcatgcgagcctgttactacagcctggccagcaaggtggaagggcccccaggcagcacacagaaggctgaggctgcctgtgcctgagctcccgctgcccaggggagccctccacnccggctcttccccaccctcagccccaccccaggtcctgcagtctggctgagccctgcttggttgtctccacacacagctgggctgtggtagggggtgtctcaggccacaccggccttgcattgcctgcctggccccctgtcctctctggctggggagcagggaggtccgggagggtgcggctgtgcagcctgtcctgggctggtggctcccggagggccctgagctgagggcattgcttacacggatgccttcccctgggccctgacattggagcctgggcatcctcaggtggtcaggcgtagatcaccagaataaacccagcttccc

SEQ ID NO: 42 - DNA, 431 bp, Homo sapiens
  modified_base (272)..(272): a, c, t, or g
  modified_base (329)..(329): a, c, t, or g
  modified_base (348)..(349): a, c, t, or g
  modified_base (352)..(352): a, c, t, or g
  modified_base (354)..(354): a, c, t, or g
  modified_base (370)..(370): a, c, t, or g
  modified_base (373)..(373): a, c, t, or g
  modified_base (377)..(377): a, c, t, or g
  modified_base (381)..(381): a, c, t, or g
  modified_base (386)..(386): a, c, t, or g
  modified_base (388)..(388): a, c, t, or g
  modified_base (391)..(391): a, c, t, or g
  modified_base (399)..(399): a, c, t, or g
  modified_base (404)..(404): a, c, t, or g
gcccctgcagcctacaaactgggcctcgggcgactcaaaataagtgctcttgggggtggctctaccccattacctcccccagccacaactcctggccttcgacttctggctgggttagccagaccctggtttctctaccctgatgttgcatgagacctggtaacagtgtctccctcccagctccttgccaaagcctctgttgagacctgggcttcttgtagccccttctccctctggccagctgcacagcctgtgggaggtgcccggcccangctgggtgtgggggaagctggtccctgctgtgggtggcgctggggacctaggggctnccttctgaggttggccttnnggncnctgggctgtatgcctnctngggngtanggaanangcngggaggantcatngggtatggtgaccccacctaagcattt

SEQ ID NO: 43 - DNA, 406 bp, Homo sapiens
tcccacgacaaaaacaagactgcctccctctcttccgggagctggtgacagccttgggcctttcagtcccaaagcggccgatgggagtctccctccgactccagatatgaacagggcccaggcctggagcgtttgctgtgccaggaggcggcagctcttctgggcagagcctgtccccgccttccctcactcttcctcatcctgcttctcttttcctcgcagatgataaaaggaatctggcattctacacctggaccatttgattgttttattttggaattggtgtatatcatgaagccttgctgaactaagttttgtgtgtatatatttaaaaaaaaaatcagtgtttaaataaagacctatgtacttaatcctttaactctgcggatagcatttggtaggtagt

SEQ ID NO: 44 - DNA, 297 bp, Homo sapiens
gccccatggcccaagttagtatatttggtttaagactcataattcttgctttggctttaaaaatcaaaccaagtgtaacctttcctgctaaaactcttcttgggtctggcagcattggtaccttgcttgcaacatcctagaagtgaggccttcacttgtgctatctttagatcataagtgatttgcttaaatttatatatttctcatgctttttataaaaccttgtttctctcttaaaagaacatattaaataagtggcttccacatttaaccatttcttttttccaactatagagt

SEQ ID NO: 45 - DNA, 411 bp, Homo sapiens
gtgtctgttgctgatgcctcaaaaagtgtgcaggtctcgactctgaagacagagttcctgccgctcctaagtgtgtcatttgtctcagagaacagcgtcgtggctgctggccatgactgctgcccaatgctctttatctacgatgaccgcggctgcctgaccttcgtctccaagttagatattccaaaacagagcatccaacgcaacatgtctgccatggaacgcttccgcaacatggacaagagagccacaactgaggaccgcaacacggccttggagacgctgcaccagaatagcatcactcaagtctctatttatgaggtggacaagcaagattgtcgcaaattttgcactactggcatcgatggagccatgacaatttgggatttcaagaccctcgagtcttccatc

SEQ ID NO: 46 - DNA, 462 bp, Homo sapiens
  modified_base (42)..(42): a, c, t, or g
  modified_base (53)..(53): a, c, t, or g
  modified_base (61)..(61): a, c, t, or g
  modified_base (65)..(65): a, c, t, or g
  modified_base (67)..(67): a, c, t, or g
  modified_base (70)..(71): a, c, t, or g
  modified_base (78)..(78): a, c, t, or g
  modified_base (85)..(85): a, c, t, or g
  modified_base (89)..(89): a, c, t, or g
  modified_base (100)..(100): a, c, t, or g
  modified_base (113)..(113): a, c, t, or g
  modified_base (118)..(118): a, c, t, or g
  modified_base (130)..(130): a, c, t, or g
  modified_base (133)..(133): a, c, t, or g
  modified_base (179)..(179): a, c, t, or g
  modified_base (190)..(190): a, c, t, or g
  modified_base (253)..(253): a, c, t, or g
  modified_base (264)..(264): a, c, t, or g
gcgcacgcctagatgccgagaaaaaagaaatgaaaacaatcncggggaagacnttttcatnccgntnatnntgggaantaagagnaggnagcggctggcnactgaaggctggnacacntgctactggatnatngtagcttttaatgttgcgcctcttcaggttcttaagggattctccnttttggttccnttttgtacacgtttggaaaataatctgcagaaacgagctgtgcttgcaaagacttcatagttncccaagaattnaaaaaaaaaaaaaaaagaattccacttgatcaacttaattccttttctttatcttccctccctcacttcccttttctcccaccctcttttccaagctgtttcgctttgcaatatattactggtaatgagttgcaggataatgcagtcataacttgttttctcctaagtatttgagttcaaaactcctgtatctaaaga

SEQ ID NO: 47 - protein, 13 aa, Artificial Sequence
  Description of Artificial Sequence: Synthetic peptide
  Mass spectrometry peptide 47
AKLPDATPTELAK

SEQ ID NO: 48 - protein, 9 aa, Artificial Sequence
  Description of Artificial Sequence: Synthetic peptide
  Mass spectrometry peptide No. 48
DATPTELAK

SEQ ID NO: 49 - protein, 7 aa, Artificial Sequence
  Description of Artificial Sequence: Synthetic peptide
  Mass spectrometry peptide No. 49
LPDATPK

SEQ ID NO: 50 - protein, 11 aa, Artificial Sequence
  Description of Artificial Sequence: Synthetic peptide
  Mass spectrometry peptide No. 50
LPDATPTELAK

SEQ ID NO: 51 - protein, 17 aa, Artificial Sequence
  Description of Artificial Sequence: Synthetic peptide
  Mass spectrometry peptide No. 51
AEQCCEETASSISLHGK

SEQ ID NO: 52 - protein, 12 aa, Artificial Sequence
  Description of Artificial Sequence: Synthetic peptide
  Mass spectrometry peptide No. 52
AIEDYINEFSVR

SEQ ID NO: 53 - protein, 8 aa, Artificial Sequence
  Description of Artificial Sequence: Synthetic peptide
  Mass spectrometry peptide No. 53
ALPTTYEK

SEQ ID NO: 54 - protein, 8 aa, Artificial Sequence
  Description of Artificial Sequence: Synthetic peptide
  Mass spectrometry peptide No. 54
APVLISQK

SEQ ID NO: 55 - protein, 8 aa, Artificial Sequence
  Description of Artificial Sequence: Synthetic peptide
  Mass spectrometry peptide No. 55
CTDAVGDR

SEQ ID NO: 56 - protein, 9 aa, Artificial Sequence
  Description of Artificial Sequence: Synthetic peptide
  Mass spectrometry peptide No. 56
CTDAVGDRR

SEQ ID NO: 57 - protein, 9 aa, Artificial Sequence
  Description of Artificial Sequence: Synthetic peptide
  Mass spectrometry peptide No. 57
DGNTLTYYR

SEQ ID NO: 58 - protein, 11 aa, Artificial Sequence
  Description of Artificial Sequence: Synthetic peptide
  Mass spectrometry peptide No. 58
DRDGNTLTYYR

SEQ ID NO: 59 - protein, 11 aa, Artificial Sequence
  Description of Artificial Sequence: Synthetic peptide
  Mass spectrometry peptide No. 59
DRVVEESELAR

SEQ ID NO: 60 - protein, 13 aa, Artificial Sequence
  Description of Artificial Sequence: Synthetic peptide
  Mass spectrometry peptide No. 60
DVVLTTTFVDDIK

SEQ ID NO: 61 - protein, 10 aa, Artificial Sequence
  Description of Artificial Sequence: Synthetic peptide
  Mass spectrometry peptide No. 61
FEGIACEISK

SEQ ID NO: 62 - protein, 5 aa, Artificial Sequence
  Description of Artificial Sequence: Synthetic peptide
  Mass spectrometry peptide No. 62
FSYSK

SEQ ID NO: 63 - protein, 8 aa, Artificial Sequence
  Description of Artificial Sequence: Synthetic peptide
  Mass spectrometry peptide No. 63
FTPTETNK

SEQ ID NO: 64 - protein, 7 aa, Artificial Sequence
  Description of Artificial Sequence: Synthetic peptide
  Mass spectrometry peptide No. 64
GEIHLGR

SEQ ID NO: 65 - protein, 11 aa, Artificial Sequence
  Description of Artificial Sequence: Synthetic peptide
  Mass spectrometry peptide No. 65
LSPIYNLVPVK

SEQ ID NO: 66 - protein, 6 aa, Artificial Sequence
  Description of Artificial Sequence: Synthetic peptide
  Mass spectrometry peptide No. 66
MFLHVK

SEQ ID NO: 67 - protein, 15 aa, Artificial Sequence
  Description of Artificial Sequence: Synthetic peptide
  Mass spectrometry peptide No. 67
NRDVVLTTTFVDDIK

SEQ ID NO: 68 - protein, 13 aa, Artificial Sequence
  Description of Artificial Sequence: Synthetic peptide
  Mass spectrometry peptide No. 68
RPWNVASLIYETK

SEQ ID NO: 69 - protein, 11 aa, Artificial Sequence
  Description of Artificial Sequence: Synthetic peptide
  Mass spectrometry peptide No. 69
SIEVFGQFNGK

SEQ ID NO: 70 - protein, 12 aa, Artificial Sequence
  Description of Artificial Sequence: Synthetic peptide
  Mass spectrometry peptide No. 70
SIEVFGQFNGKR

SEQ ID NO: 71 - protein, 6 aa, Artificial Sequence
  Description of Artificial Sequence: Synthetic peptide
  Mass spectrometry peptide No. 71
SIIQEK

SEQ ID NO: 72 - protein, 12 aa, Artificial Sequence
  Description of Artificial Sequence: Synthetic peptide
  Mass spectrometry peptide No. 72
TEHYEEQIEAFK

SEQ ID NO: 73 - protein, 6 aa, Artificial Sequence
  Description of Artificial Sequence: Synthetic peptide
  Mass spectrometry peptide No. 73
TLTYYR

SEQ ID NO: 74 - protein, 11 aa, Artificial Sequence
  Description of Artificial Sequence: Synthetic peptide
  Mass spectrometry peptide No. 74
TSNFNAAISLK

SEQ ID NO: 75 - protein, 9 aa, Artificial Sequence
  Description of Artificial Sequence: Synthetic peptide
  Mass spectrometry peptide No. 75
VVEESELAR

SEQ ID NO: 76 - protein, 6 aa, Artificial Sequence
  Description of Artificial Sequence: Synthetic peptide
  Mass spectrometry peptide No. 76
YAFELK

SEQ ID NO: 77 - protein, 7 aa, Artificial Sequence
  Description of Artificial Sequence: Synthetic peptide
  Mass spectrometry peptide No. 77
AMCQNGK

SEQ ID NO: 78 - protein, 9 aa, Artificial Sequence
  Description of Artificial Sequence: Synthetic peptide
  Mass spectrometry peptide No. 78
EATFCDFPK

SEQ ID NO: 79 - protein, 7 aa, Artificial Sequence
  Description of Artificial Sequence: Synthetic peptide
  Mass spectrometry peptide No. 79
GWSTPPK

SEQ ID NO: 80 - protein, 11 aa, Artificial Sequence
  Description of Artificial Sequence: Synthetic peptide
  Mass spectrometry peptide No. 80
INHGILYDEEK

SEQ ID NO: 81 - protein, 13 aa, Artificial Sequence
  Description of Artificial Sequence: Synthetic peptide
  Mass spectrometry peptide No. 81
ITCTEEGWSPTPK

SEQ ID NO: 82 - protein, 13 aa, Artificial Sequence
  Description of Artificial Sequence: Synthetic peptide
  Mass spectrometry peptide No. 82
LQNNENNISCVER

SEQ ID NO: 83 - protein, 26 aa, Artificial Sequence
  Description of Artificial Sequence: Synthetic peptide
  Mass spectrometry peptide No. 83
QCQNLYQLEGNNQITCRNGQWSEPPK

SEQ ID NO: 84 - protein, 5 aa, Artificial Sequence
  Description of Artificial Sequence: Synthetic peptide
  Mass spectrometry peptide No. 84
SFWTR

SEQ ID NO: 85 - protein, 20 aa, Artificial Sequence
  Description of Artificial Sequence: Synthetic peptide
  Mass spectrometry peptide No. 85
STDTSCVNPPTVQNAYIVSR

SEQ ID NO: 86 - protein, 10 aa, Artificial Sequence
  Description of Artificial Sequence: Synthetic peptide
  Mass spectrometry peptide No. 86
TGDIVEFVCK

SEQ ID NO: 87 - protein, 6 aa, Artificial Sequence
  Description of Artificial Sequence: Synthetic peptide
  Mass spectrometry peptide No. 87
WTNQQK

SEQ ID NO: 88 - protein, 4 aa, Artificial Sequence
  Description of Artificial Sequence: Synthetic peptide
  Mass spectrometry peptide No. 88
YNIK

SEQ ID NO: 89 - protein, 15 aa, Artificial Sequence
  Description of Artificial Sequence: Synthetic peptide
  Mass spectrometry peptide No. 89
EALIQFLEQVHQGIK

SEQ ID NO: 90 - protein, 9 aa, Artificial Sequence
  Description of Artificial Sequence: Synthetic peptide
  Mass spectrometry peptide No. 90
EMEMRQLQR

SEQ ID NO: 91 - protein, 6 aa, Artificial Sequence
  Description of Artificial Sequence: Synthetic peptide
  Mass spectrometry peptide No. 91
EWLGNR

SEQ ID NO: 92 - protein, 29 aa, Artificial Sequence
  Description of Artificial Sequence: Synthetic peptide
  Mass spectrometry peptide No. 92
IHILPSMNPDGYEVAAAQGPNKPGYLVGR

SEQ ID NO: 93 - protein, 9 aa, Artificial Sequence
  Description of Artificial Sequence: Synthetic peptide
  Mass spectrometry peptide No. 93
IVQLIQDTR

SEQ ID NO: 94 - protein, 5 aa, Artificial Sequence
  Description of Artificial Sequence: Synthetic peptide
  Mass spectrometry peptide No. 94
LLLFK

SEQ ID NO: 95 - protein, 14 aa, Artificial Sequence
  Description of Artificial Sequence: Synthetic peptide
  Mass spectrometry peptide No. 95
NFPDLNTYIYYNEK

SEQ ID NO: 96 - protein, 10 aa, Artificial Sequence
  Description of Artificial Sequence: Synthetic peptide
  Mass spectrometry peptide No. 96
NNANGVDLNR

SEQ ID NO: 97 - protein, 6 aa, Artificial Sequence
  Description of Artificial Sequence: Synthetic peptide
  Mass spectrometry peptide No. 97
QLQRGP

SEQ ID NO: 98 - protein, 10 aa, Artificial Sequence
  Description of Artificial Sequence: Synthetic peptide
  Mass spectrometry peptide No. 98
RSIPQVSPVR

SEQ ID NO: 99 - protein, 9 aa, Artificial Sequence
  Description of Artificial Sequence: Synthetic peptide
  Mass spectrometry peptide No. 99
SIPQVSPVR

SEQ ID NO: 100 - protein, 10 aa, Artificial Sequence
  Description of Artificial Sequence: Synthetic peptide
  Mass spectrometry peptide No. 100
TASTPTPDDK

SEQ ID NO: 101 - protein, 10 aa, Artificial Sequence
  Description of Artificial Sequence: Synthetic peptide
  Mass spectrometry peptide No. 101
VQNECPGITR

SEQ ID NO: 102 - protein, 6 aa, Artificial Sequence
  Description of Artificial Sequence: Synthetic peptide
  Mass spectrometry peptide No. 102
VYSIGR

SEQ ID NO: 103 - protein, 6 aa, Artificial Sequence
  Description of Artificial Sequence: Synthetic peptide
  Mass spectrometry peptide No. 103
YDDLVR

SEQ ID NO: 104 - protein, 13 aa, Artificial Sequence
  Description of Artificial Sequence: Synthetic peptide
  Mass spectrometry peptide No. 104
YVGNMHGNEALGR

SEQ ID NO: 105 - protein, 14 aa, Artificial Sequence
  Description of Artificial Sequence: Synthetic peptide
  Mass spectrometry peptide No. 105
DHVITNMNNNYEPR

SEQ ID NO: 106 - protein, 15 aa, Artificial Sequence
  Description of Artificial Sequence: Synthetic peptide
  Mass spectrometry peptide No. 106
FDHVITNMNNNYEPR

SEQ ID NO: 107 - protein, 11 aa, Artificial Sequence
  Description of Artificial Sequence: Synthetic peptide
  Mass spectrometry peptide No. 107
GDPGIPGNPGK

SEQ ID NO: 108 - protein, 12 aa, Artificial Sequence
  Description of Artificial Sequence: Synthetic peptide
  Mass spectrometry peptide No. 108
GGPGAPGAPGPK

SEQ ID NO: 109 - protein, 9 aa, Artificial Sequence
  Description of Artificial Sequence: Synthetic peptide
  Mass spectrometry peptide No. 109
GNLCVNLMR

SEQ ID NO: 110 - protein, 7 aa, Artificial Sequence
  Description of Artificial Sequence: Synthetic peptide
  Mass spectrometry peptide No. 110
IAFSATR

SEQ ID NO: 111 - protein, 14 aa, Artificial Sequence
  Description of Artificial Sequence: Synthetic peptide
  Mass spectrometry peptide No. 111
LEQGENVFLQATDK

SEQ ID NO: 112 - protein, 8 aa, Artificial Sequence
  Description of Artificial Sequence: Synthetic peptide
  Mass spectrometry peptide No. 112
MNNNYEPR

SEQ ID NO: 113 - protein, 11 aa, Artificial Sequence
  Description of Artificial Sequence: Synthetic peptide
  Mass spectrometry peptide No. 113
NVPLRRDQTIR

SEQ ID NO: 114 - protein, 7 aa, Artificial Sequence
  Description of Artificial Sequence: Synthetic peptide
  Mass spectrometry peptide No. 114
TINVPLR

SEQ ID NO: 115 - protein, 8 aa, Artificial Sequence
  Description of Artificial Sequence: Synthetic peptide
  Mass spectrometry peptide No. 115
TINVPLRR

SEQ ID NO: 116 - protein, 14 aa, Artificial Sequence
  Description of Artificial Sequence: Synthetic peptide
  Mass spectrometry peptide No. 116
VPGLYYFTYHASSR

SEQ ID NO: 117 - protein, 12 aa, Artificial Sequence
  Description of Artificial Sequence: Synthetic peptide
  Mass spectrometry peptide No. 117
EFTPQMQAAYQK

SEQ ID NO: 118 - protein, 19 aa, Artificial Sequence
  Description of Artificial Sequence: Synthetic peptide
  Mass spectrometry peptide No. 118
FFESFGDLSTPDAVMGNPK

SEQ ID NO: 119 - protein, 13 aa, Artificial Sequence
  Description of Artificial Sequence: Synthetic peptide
  Mass spectrometry peptide No. 119
GTFATLSELHCDK

SEQ ID NO: 120 - protein, 9 aa, Artificial Sequence
  Description of Artificial Sequence: Synthetic peptide
  Mass spectrometry peptide No. 120
LHVDPENFR

SEQ ID NO: 121 - protein, 12 aa, Artificial Sequence
  Description of Artificial Sequence: Synthetic peptide
  Mass spectrometry peptide No. 121
LLGNVLVCVLAR

SEQ ID NO: 122 - protein, 10 aa, Artificial Sequence
  Description of Artificial Sequence: Synthetic peptide
  Mass spectrometry peptide No. 122
LLVVYPWTQR

SEQ ID NO: 123 - protein, 9 aa, Artificial Sequence
  Description of Artificial Sequence: Synthetic peptide
  Mass spectrometry peptide No. 123
SAVTALWGK

SEQ ID NO: 124 - protein, 11 aa, Artificial Sequence
  Description of Artificial Sequence: Synthetic peptide
  Mass spectrometry peptide No. 124
VDEVGGEALGR

SEQ ID NO: 125 - protein, 16 aa, Artificial Sequence
  Description of Artificial Sequence: Synthetic peptide
  Mass spectrometry peptide No. 125
VLGAFSDGLAHLDNLK

SEQ ID NO: 126 - protein, 13 aa, Artificial Sequence
  Description of Artificial Sequence: Synthetic peptide
  Mass spectrometry peptide No. 126
VNVDEVGGEALGR

SEQ ID NO: 127 - protein, 12 aa, Artificial Sequence
  Description of Artificial Sequence: Synthetic peptide
  Mass spectrometry peptide No. 127
VVAGVANALAHK

SEQ ID NO: 128 - protein, 8 aa, Artificial Sequence
  Description of Artificial Sequence: Synthetic peptide
  Mass spectrometry peptide No. 128
AAFCYQIR

SEQ ID NO: 129 - protein, 6 aa, Artificial Sequence
  Description of Artificial Sequence: Synthetic peptide
  Mass spectrometry peptide No. 129
CDLFQK

SEQ ID NO: 130 - protein, 7 aa, Artificial Sequence
  Description of Artificial Sequence: Synthetic peptide
  Mass spectrometry peptide No. 130
DYCALRR

SEQ ID NO: 131 - protein, 7 aa, Artificial Sequence
  Description of Artificial Sequence: Synthetic peptide
  Mass spectrometry peptide No. 131
EFCDLPR

SEQ ID NO: 132 - protein, 8 aa, Artificial Sequence
  Description of Artificial Sequence: Synthetic peptide
  Mass spectrometry peptide No. 132
EQWILTAR

SEQ ID NO: 133 - protein, 18 aa, Artificial Sequence
  Description of Artificial Sequence: Synthetic peptide
  Mass spectrometry peptide No. 133
GTMATTVGGLPCQAWSHK

SEQ ID NO: 134 - protein, 20 aa, Artificial Sequence
  Description of Artificial Sequence: Synthetic peptide
  Mass spectrometry peptide No. 134
LRVVGGHPGNSPWTVSLRNR

SEQ ID NO: 135 - protein, 9 aa, Artificial Sequence
  Description of Artificial Sequence: Synthetic peptide
  Mass spectrometry peptide No. 135
NGLEENFCR

SEQ ID NO: 136 - protein, 19 aa, Artificial Sequence
  Description of Artificial Sequence: Synthetic peptide
  Mass spectrometry peptide No. 136
NPDGDPGGPWCYTTDPAVR

SEQ ID NO: 137 - protein, 10 aa, Artificial Sequence
  Description of Artificial Sequence: Synthetic peptide
  Mass spectrometry peptide No. 137
SPLNDFQVLR

SEQ ID NO: 138 - protein, 9 aa, Artificial Sequence
  Description of Artificial Sequence: Synthetic peptide
  Mass spectrometry peptide No. 138
TPFDYCALR

SEQ ID NO: 139 - protein, 16 aa, Artificial Sequence
  Description of Artificial Sequence: Synthetic peptide
  Mass spectrometry peptide No. 139
VVGGHPGNSPWTVSLR

SEQ ID NO: 140 - protein, 7 aa, Artificial Sequence
  Description of Artificial Sequence: Synthetic peptide
  Mass spectrometry peptide No. 140
WPAVFTR

SEQ ID NO: 141 - protein, 6 aa, Artificial Sequence
  Description of Artificial Sequence: Synthetic peptide
  Mass spectrometry peptide No. 141
YTPTLR

SEQ ID NO: 142 - protein, 11 aa, Artificial Sequence
  Description of Artificial Sequence: Synthetic peptide
  Mass spectrometry peptide No. 142
ASSAGLGPVVR

SEQ ID NO: 143 - protein, 9 aa, Artificial Sequence
  Description of Artificial Sequence: Synthetic peptide
  Mass spectrometry peptide No. 143
CQPSPDEAR

SEQ ID NO: 144 - protein, 10 aa, Artificial Sequence
  Description of Artificial Sequence: Synthetic peptide
  Mass spectrometry peptide No. 144
EMEDTLNHLK

SEQ ID NO: 145 - protein, 8 aa, Artificial Sequence
  Description of Artificial Sequence: Synthetic peptide
  Mass spectrometry peptide No. 145
ETEYGPCR

SEQ ID NO: 146 - protein, 8 aa, Artificial Sequence
  Description of Artificial Sequence: Synthetic peptide
  Mass spectrometry peptide No. 146
FLNVLSPR

SEQ ID NO: 147 - protein, 12 aa, Artificial Sequence
  Description of Artificial Sequence: Synthetic peptide
  Mass spectrometry peptide No. 147
GASSAGLGPVVR

SEQ ID NO: 148 - protein, 4 aa, Artificial Sequence
  Description of Artificial Sequence: Synthetic peptide
  Mass spectrometry peptide No. 148
GFYK

SEQ ID NO: 149 - protein, 9 aa, Artificial Sequence
  Description of Artificial Sequence: Synthetic peptide
  Mass spectrometry peptide No. 149
PLQALLDGR

SEQ ID NO: 150 - protein, 15 aa, Artificial Sequence
  Description of Artificial Sequence: Synthetic peptide
  Mass spectrometry peptide No. 150
SAGSVESPSVSSTHR

SEQ ID NO: 151 - protein, 11 aa, Artificial Sequence
  Description of Artificial Sequence: Synthetic peptide
  Mass spectrometry peptide No. 151
YGQPLPGYTTK

SEQ ID NO: 152 - protein, 6 aa, Artificial Sequence
  Description of Artificial Sequence: Synthetic peptide
  Mass spectrometry peptide No. 152
DAQGLR

SEQ ID NO: 153 - protein, 11 aa, Artificial Sequence
  Description of Artificial Sequence: Synthetic peptide
  Mass spectrometry peptide No. 153
ELDSTGTPTGK

SEQ ID NO: 154 - protein, 12 aa, Artificial Sequence
  Description of Artificial Sequence: Synthetic peptide
  Mass spectrometry peptide No. 154
GKEYFAIDNSGR

SEQ ID NO: 155 - protein, 14 aa, Artificial Sequence
  Description of Artificial Sequence: Synthetic peptide
  Mass spectrometry peptide No. 155
RDWIWNQMHIDEEK

SEQ ID NO: 156 - protein, 16 aa, Artificial Sequence
  Description of Artificial Sequence: Synthetic peptide
  Mass spectrometry peptide No. 156
VHFLPVVISDNGMPSR

SEQ ID NO: 157 - protein, 8 aa, Artificial Sequence
  Description of Artificial Sequence: Synthetic peptide
  Mass spectrometry peptide No. 157
YEIVVEAR

SEQ ID NO: 158 - protein, 6 aa, Artificial Sequence
  Description of Artificial Sequence: Synthetic peptide
  Mass spectrometry peptide No. 158
YGQFDR

SEQ ID NO: 159 - protein, 9 aa, Artificial Sequence
  Description of Artificial Sequence: Synthetic peptide
  Mass spectrometry peptide No. 159
YMSPPAGNR

SEQ ID NO: 160 - protein, 10 aa, Artificial Sequence
  Description of Artificial Sequence: Synthetic peptide
  Mass spectrometry peptide No. 160
YTFVVPEDTR

SEQ ID NO: 161 - protein, 7 aa, Artificial Sequence
  Description of Artificial Sequence: Synthetic peptide
  Mass spectrometry peptide No. 161
AAIQALR

SEQ ID NO: 162 - protein, 22 aa, Artificial Sequence
  Description of Artificial Sequence: Synthetic peptide
  Mass spectrometry peptide No. 162
AALTELSLGSAYQAMILGVDSK

SEQ ID NO: 163 - protein, 13 aa, Artificial Sequence
  Description of Artificial Sequence: Synthetic peptide
  Mass spectrometry peptide No. 163
AASGTTGTYQEWK

SEQ ID NO: 164 - protein, 23 aa, Artificial Sequence
  Description of Artificial Sequence: Synthetic peptide
  Mass spectrometry peptide No. 164
ADSVVDLLSYNVQGSGETTYDHK

SEQ ID NO: 165 - protein, 6 aa, Artificial Sequence
  Description of Artificial Sequence: Synthetic peptide
  Mass spectrometry peptide No. 165
AEFTGR

SEQ ID NO: 166 - protein, 13 aa, Artificial Sequence
  Description of Artificial Sequence: Synthetic peptide
  Mass spectrometry peptide No. 166
AEPLAFTFSHDYK

SEQ ID NO: 167 - protein, 10 aa, Artificial Sequence
  Description of Artificial Sequence: Synthetic peptide
  Mass spectrometry peptide No. 167
AEWVYQGAIR

SEQ ID NO: 168 - protein, 8 aa, Artificial Sequence
  Description of Artificial Sequence: Synthetic peptide
  Mass spectrometry peptide No. 168
AFTDLHLR

SEQ ID NO: 169 - protein, 6 aa, Artificial Sequence
  Description of Artificial Sequence: Synthetic peptide
  Mass spectrometry peptide No. 169
AIQSLK

SEQ ID NO: 170 - protein, 7 aa, Artificial Sequence
  Description of Artificial Sequence: Synthetic peptide
  Mass spectrometry peptide No. 170
ALVDTLK

SEQ ID NO: 171 - protein, 13 aa, Artificial Sequence
  Description of Artificial Sequence: Synthetic peptide
  Mass spectrometry peptide No. 171
ALVEQGFTVPEIK

SEQ ID NO: 172 - protein, 15 aa, Artificial Sequence
  Description of Artificial Sequence: Synthetic peptide
  Mass spectrometry peptide No. 172
ALYWVNGQVPDGVSK

SEQ ID NO: 173 - protein, 6 aa, Artificial Sequence
  Description of Artificial Sequence: Synthetic peptide
  Mass spectrometry peptide No. 173
ANLFNK

SEQ ID NO: 174 - protein, 7 aa, Artificial Sequence
  Description of Artificial Sequence: Synthetic peptide
  Mass spectrometry peptide No. 174
AQLSNPK

SEQ ID NO: 175 - protein, 21 aa, Artificial Sequence
  Description of Artificial Sequence: Synthetic peptide
  Mass spectrometry peptide No. 175
AQNLYQELLTQEGQASFQGLK

SEQ ID NO: 176 - protein, 19 aa, Artificial Sequence
  Description of Artificial Sequence: Synthetic peptide
  Mass spectrometry peptide No. 176
ASGSLPYTQTLQDHLNSLK

SEQ ID NO: 177 - protein, 16 aa, Artificial Sequence
  Description of Artificial Sequence: Synthetic peptide
  Mass spectrometry peptide No. 177
ATFQTPDFIVPLTDLR

SEQ ID NO: 178 - protein, 11 aa, Artificial Sequence
  Description of Artificial Sequence: Synthetic peptide
  Mass spectrometry peptide No. 178
ATGVLYDYVNK

SEQ ID NO: 179 - protein, 15 aa, Artificial Sequence
  Description of Artificial Sequence: Synthetic peptide
  Mass spectrometry peptide No. 179
AVSMPSFSILGSDVR

SEQ ID NO: 180 - protein, 13 aa, Artificial Sequence
  Description of Artificial Sequence: Synthetic peptide
  Mass spectrometry peptide No. 180
CVQSTKPSLMIQK

SEQ ID NO: 181 - protein, 14 aa, Artificial Sequence
  Description of Artificial Sequence: Synthetic peptide
  Mass spectrometry peptide No. 181
DAVEKPQEFTIVAF

SEQ ID NO: 182 - protein, 16 aa, Artificial Sequence
  Description of Artificial Sequence: Synthetic peptide
  Mass spectrometry peptide No. 182
DAVEKPQEFTIVAFVK

SEQ ID NO: 183 - protein, 7 aa, Artificial Sequence
  Description of Artificial Sequence: Synthetic peptide
  Mass spectrometry peptide No. 183
DFSLWEK

SEQ ID NO: 184 - protein, 20 aa, Artificial Sequence
  Description of Artificial Sequence: Synthetic peptide
  Mass spectrometry peptide No. 184
DKDQEVLLQTFLDDASPGDK

SEQ ID NO: 185 - protein, 10 aa, Artificial Sequence
  Description of Artificial Sequence: Synthetic peptide
  Mass spectrometry peptide No. 185
DKIGVELTGR

SEQ ID NO: 186 - protein, 7 aa, Artificial Sequence
  Description of Artificial Sequence: Synthetic peptide
  Mass spectrometry peptide No. 186
DLGQCDR

SEQ ID NO: 187 - protein, 11 aa, Artificial Sequence
  Description of Artificial Sequence: Synthetic peptide
  Mass spectrometry peptide No. 187
DLKVEDIPLAR

SEQ ID NO: 188 - protein, 9 aa, Artificial Sequence
  Description of Artificial Sequence: Synthetic peptide
  Mass spectrometry peptide No. 188
DNVFDGLVR

SEQ ID NO: 189 - protein, 14 aa, Artificial Sequence
  Description of Artificial Sequence: Synthetic peptide
  Mass spectrometry peptide No. 189
DPNTGRLNGESNLR

SEQ ID NO: 190 - protein, 5 aa, Artificial Sequence
  Description of Artificial Sequence: Synthetic peptide
  Mass spectrometry peptide No. 190
DYVNK

SEQ ID NO: 191 - protein, 15 aa, Artificial Sequence
  Description of Artificial Sequence: Synthetic peptide
  Mass spectrometry peptide No. 191
EALKESQLPTVMDFR

SEQ ID NO: 192 - protein, 7 aa, Artificial Sequence
  Description of Artificial Sequence: Synthetic peptide
  Mass spectrometry peptide No. 192
EAQEVFK

SEQ ID NO: 193 - protein, 14 aa, Artificial Sequence
  Description of Artificial Sequence: Synthetic peptide
  Mass spectrometry peptide No. 193
EEEMLENVSLVCPK

SEQ ID NO: 194 - protein, 9 aa, Artificial Sequence
  Description of Artificial Sequence: Synthetic peptide
  Mass spectrometry peptide No. 194
EELCTMFIR

SEQ ID NO: 195 - protein, 11 aa, Artificial Sequence
  Description of Artificial Sequence: Synthetic peptide
  Mass spectrometry peptide No. 195
EFQVPTFTIPK

SEQ ID NO: 196 - protein, 7 aa, Artificial Sequence
  Description of Artificial Sequence: Synthetic peptide
  Mass spectrometry peptide No. 196
EIFNMAR

SEQ ID NO: 197 - protein, 6 aa, Artificial Sequence
  Description of Artificial Sequence: Synthetic peptide
  Mass spectrometry peptide No. 197
EIQIYK

SEQ ID NO: 198 - protein, 11 aa, Artificial Sequence
  Description of Artificial Sequence: Synthetic peptide
  Mass spectrometry peptide No. 198
ENFAGEATLQR

SEQ ID NO: 199 - protein, 8 aa, Artificial Sequence
  Description of Artificial Sequence: Synthetic peptide
  Mass spectrometry peptide No. 199
EQHLFLPF

SEQ ID NO: 200 - protein, 11 aa, Artificial Sequence
  Description of Artificial Sequence: Synthetic peptide
  Mass spectrometry peptide No. 200
EQHLFLPFSYN

SEQ ID NO: 201 - protein, 9 aa, Artificial Sequence
  Description of Artificial Sequence: Synthetic peptide
  Mass spectrometry peptide No. 201
ESDEETQIK

SEQ ID NO: 202 - protein, 11 aa, Artificial Sequence
  Description of Artificial Sequence: Synthetic peptide
  Mass spectrometry peptide No. 202
ESQLPTVMDFR

SEQ ID NO: 203 - protein, 7 aa, Artificial Sequence
  Description of Artificial Sequence: Synthetic peptide
  Mass spectrometry peptide No. 203
ETLEDTR

SEQ ID NO: 204 - protein, 9 aa, Artificial Sequence
  Description of Artificial Sequence: Synthetic peptide
  Mass spectrometry peptide No. 204
ETLEDTRDR

SEQ ID NO: 205 - protein, 10 aa, Artificial Sequence
  Description of Artificial Sequence: Synthetic peptide
  Mass spectrometry peptide No. 205
EVYGFNPEGK

SEQ ID NO: 206 - protein, 17 aa, Artificial Sequence
  Description of Artificial Sequence: Synthetic peptide
  Mass spectrometry peptide No. 206
EYSGTIASEANTYLNSK

SEQ ID NO: 207 - protein, 19 aa, Artificial Sequence
  Description of Artificial Sequence: Synthetic peptide
  Mass spectrometry peptide No. 207
FDHTNSLNIAGLSLDFSSK

SEQ ID NO: 208 - protein, 7 aa, Artificial Sequence
  Description of Artificial Sequence: Synthetic peptide
  Mass spectrometry peptide No. 208
FFGEGTK

SEQ ID NO: 209 - protein, 6 aa, Artificial Sequence
  Description of Artificial Sequence: Synthetic peptide
  Mass spectrometry peptide No. 209
FIAESK

SEQ ID NO: 210 - protein, 7 aa, Artificial Sequence
  Description of Artificial Sequence: Synthetic peptide
  Mass spectrometry peptide No. 210
FIIPSPK

SEQ ID NO: 211 - protein, 7 aa, Artificial Sequence
  Description of Artificial Sequence: Synthetic peptide
  Mass spectrometry peptide No. 211
FLDMLIK

SEQ ID NO: 212 - protein, 7 aa, Artificial Sequence
  Description of Artificial Sequence: Synthetic peptide
  Mass spectrometry peptide No. 212
FLDSNIK

SEQ ID NO: 213 - protein, 5 aa, Artificial Sequence
  Description of Artificial Sequence: Synthetic peptide
  Mass spectrometry peptide No. 213
FNQYK

SEQ ID NO: 214 - protein, 7 aa, Artificial Sequence
  Description of Artificial Sequence: Synthetic peptide
  Mass spectrometry peptide No. 214
FPDSVNK

SEQ ID NO: 215 - protein, 9 aa, Artificial Sequence
  Description of Artificial Sequence: Synthetic peptide
  Mass spectrometry peptide No. 215
FPEVDVLTK

SEQ ID NO: 216 - protein, 6 aa, Artificial Sequence
  Description of Artificial Sequence: Synthetic peptide
  Mass spectrometry peptide No. 216
FQFPGK

SEQ ID NO: 217 - protein, 12 aa, Artificial Sequence
  Description of Artificial Sequence: Synthetic peptide
  Mass spectrometry peptide No. 217
FQFPGKPGIYTR

SEQ ID NO: 218 - protein, 9 aa, Artificial Sequence
  Description of Artificial Sequence: Synthetic peptide
  Mass spectrometry peptide No. 218
FRETLEDTR

SEQ ID NO: 219 - protein, 6 aa, Artificial Sequence
  Description of Artificial Sequence: Synthetic peptide
  Mass spectrometry peptide No. 219
FSLDGK

SEQ ID NO: 220 - protein, 18 aa, Artificial Sequence
  Description of Artificial Sequence: Synthetic peptide
  Mass spectrometry peptide No. 220
FSVPAGIVIPSFQALTAR

SEQ ID NO: 221 - protein, 6 aa, Artificial Sequence
  Description of Artificial Sequence: Synthetic peptide
  Mass spectrometry peptide No. 221
FVRKYR

SEQ ID NO: 222 - protein, 9 aa, Artificial Sequence
  Description of Artificial Sequence: Synthetic peptide
  Mass spectrometry peptide No. 222
FVTQAEGAK

SEQ ID NO: 223 - protein, 9 aa, Artificial Sequence
  Description of Artificial Sequence: Synthetic peptide
  Mass spectrometry peptide No. 223
GAYQNNEIK

SEQ ID NO: 224 - protein, 12 aa, Artificial Sequence
  Description of Artificial Sequence: Synthetic peptide
  Mass spectrometry peptide No. 224
GFEPTLEALFGK

SEQ ID NO: 225 - protein, 16 aa, Artificial Sequence
  Description of Artificial Sequence: Synthetic peptide
  Mass spectrometry peptide No. 225
GIISALLVPPETEEAK

SEQ ID NO: 226 - protein, 26 aa, Artificial Sequence
  Description of Artificial Sequence: Synthetic peptide
  Mass spectrometry peptide No. 226
GISTSAASPAVGTVGMDMDEDDDFSK

SEQ ID NO: 227 - protein, 9 aa, Artificial Sequence
  Description of Artificial Sequence: Synthetic peptide
  Mass spectrometry peptide No. 227
GMALFGEGK

SEQ ID NO: 228 - protein, 11 aa, Artificial Sequence
  Description of Artificial Sequence: Synthetic peptide
  Mass spectrometry peptide No. 228
GNVATEISTER

SEQ ID NO: 229 - protein, 9 aa, Artificial Sequence
  Description of Artificial Sequence: Synthetic peptide
  Mass spectrometry peptide No. 229
GQVPDGVSK

SEQ ID NO: 230 - protein, 9 aa, Artificial Sequence
  Description of Artificial Sequence: Synthetic peptide
  Mass spectrometry peptide No. 230
GTYGLSCQR

SEQ ID NO: 231 - protein, 15 aa, Artificial Sequence
  Description of Artificial Sequence: Synthetic peptide
  Mass spectrometry peptide No. 231
GTYGLSCQRDPNTGR

SEQ ID NO: 232 - protein, 7 aa, Artificial Sequence
  Description of Artificial Sequence: Synthetic peptide
  Mass spectrometry peptide No. 232
GVISIPR

SEQ ID NO: 233 - protein, 13 aa, Artificial Sequence
  Description of Artificial Sequence: Synthetic peptide
  Mass spectrometry peptide No. 233
GVISIPRLQAEAR

SEQ ID NO: 234 - protein, 6 aa, Artificial Sequence
  Description of Artificial Sequence: Synthetic peptide
  Mass spectrometry peptide No. 234
GYVFPK

SEQ ID NO: 235 - protein, 9 aa, Artificial Sequence
  Description of Artificial Sequence: Synthetic peptide
  Mass spectrometry peptide No. 235
HINIDQFVR

SEQ ID NO: 236 - protein, 15 aa, Artificial Sequence
  Description of Artificial Sequence: Synthetic peptide
  Mass spectrometry peptide No. 236
HIYAISSAALSASYK

SEQ ID NO: 237 - protein, 14 aa, Artificial Sequence
  Description of Artificial Sequence: Synthetic peptide
  Mass spectrometry peptide No. 237
HLIDSLIDFLNFPR

SEQ ID NO: 238 - protein, 8 aa, Artificial Sequence
  Description of Artificial Sequence: Synthetic peptide
  Mass spectrometry peptide No. 238
HRHSITNP

SEQ ID NO: 239 - protein, 22 aa, Artificial Sequence
  Description of Artificial Sequence: Synthetic peptide
  Mass spectrometry peptide No. 239
IADFELPTIIVPEQTIEIPSIK

SEQ ID NO: 240 - protein, 13 aa, Artificial Sequence
  Description of Artificial Sequence: Synthetic peptide
  Mass spectrometry peptide No. 240
IAELSATAQEIIK

SEQ ID NO: 241 - protein, 13 aa, Artificial Sequence
  Description of Artificial Sequence: Synthetic peptide
  Mass spectrometry peptide No. 241
IAIANIIDEIIEK

SEQ ID NO: 242 - protein, 10 aa, Artificial Sequence
  Description of Artificial Sequence: Synthetic peptide
  Mass spectrometry peptide No. 242
IDDIWNLEVK

SEQ ID NO: 243 - protein, 6 aa, Artificial Sequence
  Description of Artificial Sequence: Synthetic peptide
  Mass spectrometry peptide No. 243
IDGQFR

SEQ ID NO: 244 - protein, 9 aa, Artificial Sequence
  Description of Artificial Sequence: Synthetic peptide
  Mass spectrometry peptide No. 244
IEDGTLASK

SEQ ID NO: 245 - protein, 15 aa, Artificial Sequence
  Description of Artificial Sequence: Synthetic peptide
  Mass spectrometry peptide No. 245
IEGNLIFDPNNYLPK

SEQ ID NO: 246 - protein, 10 aa, Artificial Sequence
  Description of Artificial Sequence: Synthetic peptide
  Mass spectrometry peptide No. 246
IEIPLPFGGK

SEQ ID NO: 247 - protein, 15 aa, Artificial Sequence
  Description of Artificial Sequence: Synthetic peptide
  Mass spectrometry peptide No. 247
IGQDGISTSATTNLK

SEQ ID NO: 248 - protein, 8 aa, Artificial Sequence
  Description of Artificial Sequence: Synthetic peptide
  Mass spectrometry peptide No. 248
IGVELTGR

SEQ ID NO: 249 - protein, 18 aa, Artificial Sequence
  Description of Artificial Sequence: Synthetic peptide
  Mass spectrometry peptide No. 249
IHSGSFQSQVELSNDQEK

SEQ ID NO: 250 - protein, 10 aa, Artificial Sequence
  Description of Artificial Sequence: Synthetic peptide
  Mass spectrometry peptide No. 250
IISDYHQQFR

SEQ ID NO: 251 - protein, 9 aa, Artificial Sequence
  Description of Artificial Sequence: Synthetic peptide
  Mass spectrometry peptide No. 251
IKGVISIPR

SEQ ID NO: 252 - protein, 19 aa, Artificial Sequence
  Description of Artificial Sequence: Synthetic peptide
  Mass spectrometry peptide No. 252
ILGEELGFASLHDLQLLGK

SEQ ID NO: 253 - protein, 7 aa, Artificial Sequence
  Description of Artificial Sequence: Synthetic peptide
  Mass spectrometry peptide No. 253
ILGVDSK

SEQ ID NO: 254 - protein, 6 aa, Artificial Sequence
  Description of Artificial Sequence: Synthetic peptide
  Mass spectrometry peptide No. 254
INFNEK

SEQ ID NO: 255 - protein, 8 aa, Artificial Sequence
  Description of Artificial Sequence: Synthetic peptide
  Mass spectrometry peptide No. 255
INIDQFVR

SEQ ID NO: 256 - protein, 12 aa, Artificial Sequence
  Description of Artificial Sequence: Synthetic peptide
  Mass spectrometry peptide No. 256
INNQLTLDSNTK

SEQ ID NO: 257 - protein, 7 aa, Artificial Sequence
  Description of Artificial Sequence: Synthetic peptide
  Mass spectrometry peptide No. 257
INPLALK

SEQ ID NO: 258 - protein, 9 aa, Artificial Sequence
  Description of Artificial Sequence: Synthetic peptide
  Mass spectrometry peptide No. 258
IPSVQINFK

SEQ ID NO: 259 - protein, 6 aa, Artificial Sequence
  Description of Artificial Sequence: Synthetic peptide
  Mass spectrometry peptide No. 259
IQIQEK

SEQ ID NO: 260 - protein, 11 aa, Artificial Sequence
  Description of Artificial Sequence: Synthetic peptide
  Mass spectrometry peptide No. 260
IQIQEKLQQLK

SEQ ID NO: 261 - protein, 14 aa, Artificial Sequence
  Description of Artificial Sequence: Synthetic peptide
  Mass spectrometry peptide No. 261
ITENDIQIALDDAK

SEQ ID NO: 262 - protein, 7 aa, Artificial Sequence
  Description of Artificial Sequence: Synthetic peptide
  Mass spectrometry peptide No. 262
ITLPDFR

SEQ ID NO: 263 - protein, 14 aa, Artificial Sequence
  Description of Artificial Sequence: Synthetic peptide
  Mass spectrometry peptide No. 263
IVQILPWEQNEQVK

SEQ ID NO: 264 - protein, 10 aa, Artificial Sequence
  Description of Artificial Sequence: Synthetic peptide
  Mass spectrometry peptide No. 264
IYSLWEHSTK

SEQ ID NO: 265 - protein, 11 aa, Artificial Sequence
  Description of Artificial Sequence: Synthetic peptide
  Mass spectrometry peptide No. 265
KLTISEQNIQR

SEQ ID NO: 266 - protein, 21 aa, Artificial Sequence
  Description of Artificial Sequence: Synthetic peptide
  Mass spectrometry peptide No. 266
KYTYNYEAESSSGVPGTADSR

SEQ ID NO: 267 - protein, 9 aa, Artificial Sequence
  Description of Artificial Sequence: Synthetic peptide
  Mass spectrometry peptide No. 267
LAAYLMLMR

SEQ ID NO: 268 - protein, 7 aa, Artificial Sequence
  Description of Artificial Sequence: Synthetic peptide
  Mass spectrometry peptide No. 268
LAIPEGK

SEQ ID NO: 269 - protein, 14 aa, Artificial Sequence
  Description of Artificial Sequence: Synthetic peptide
  Mass spectrometry peptide No. 269
LALWGEHTGQLYSK

SEQ ID NO: 270 - protein, 9 aa, Artificial Sequence
  Description of Artificial Sequence: Synthetic peptide
  Mass spectrometry peptide No. 270
LDDASPGDK

SEQ ID NO: 271 - protein, 10 aa, Artificial Sequence
  Description of Artificial Sequence: Synthetic peptide
  Mass spectrometry peptide No. 271
LDFSSQADLR

SEQ ID NO: 272 - protein, 9 aa, Artificial Sequence
  Description of Artificial Sequence: Synthetic peptide
  Mass spectrometry peptide No. 272
LDNIYSSDK

SEQ ID NO: 273 - protein, 9 aa, Artificial Sequence
  Description of Artificial Sequence: Synthetic peptide
  Mass spectrometry peptide No. 273
LDVTTSIGR

SEQ ID NO: 274 - protein, 6 aa, Artificial Sequence
  Description of Artificial Sequence: Synthetic peptide
  Mass spectrometry peptide No. 274
LEDTPK

SEQ ID NO: 275 - protein, 14 aa, Artificial Sequence
  Description of Artificial Sequence: Synthetic peptide
  Mass spectrometry peptide No. 275
LEIQSQVDSQHVGH

SEQ ID NO: 276 - protein, 20 aa, Artificial Sequence
  Description of Artificial Sequence: Synthetic peptide
  Mass spectrometry peptide No. 276
LEIQSQVDSQHVGHSVLTAK

SEQ ID NO: 277 - protein, 24 aa, Artificial Sequence
  Description of Artificial Sequence: Synthetic peptide
  Mass spectrometry peptide No. 277
LELELRPTGEIEQYSVSATYELQR

SEQ ID NO: 278 - protein, 5 aa, Artificial Sequence
  Description of Artificial Sequence: Synthetic peptide
  Mass spectrometry peptide No. 278
LEPLK

SEQ ID NO: 279 - protein, 13 aa, Artificial Sequence
  Description of Artificial Sequence: Synthetic peptide
  Mass spectrometry peptide No. 279
LEVANMQAELVAK

SEQ ID NO: 280 - protein, 18 aa, Artificial Sequence
  Description of Artificial Sequence: Synthetic peptide
  Mass spectrometry peptide No. 280
LEVLNFDFQANAQLSNPK

SEQ ID NO: 281 - protein, 8 aa, Artificial Sequence
  Description of Artificial Sequence: Synthetic peptide
  Mass spectrometry peptide No. 281
LGNNPVSK

SEQ ID NO: 282 - protein, 8 aa, Artificial Sequence
  Description of Artificial Sequence: Synthetic peptide
  Mass spectrometry peptide No. 282
LHVAGNLK

SEQ ID NO: 283 - protein, 9 aa, Artificial Sequence
  Description of Artificial Sequence: Synthetic peptide
  Mass spectrometry peptide No. 283
LIDVISMYR

SEQ ID NO: 284 - protein, 11 aa, Artificial Sequence
  Description of Artificial Sequence: Synthetic peptide
  Mass spectrometry peptide No. 284
LIVAMSSWLQK

SEQ ID NO: 285 - protein, 7 aa, Artificial Sequence
  Description of Artificial Sequence: Synthetic peptide
  Mass spectrometry peptide No. 285
LLLMGAR

SEQ ID NO: 286 - protein, 18 aa, Artificial Sequence
  Description of Artificial Sequence: Synthetic peptide
  Mass spectrometry peptide No. 286
LLLQMDSSATAYGSTVSK

SEQ ID NO: 287 - protein, 15 aa, Artificial Sequence
  Description of Artificial Sequence: Synthetic peptide
  Mass spectrometry peptide No. 287
LLSGGNTLHLVSTTK

SEQ ID NO: 288 - protein, 7 aa, Artificial Sequence
  Description of Artificial Sequence: Synthetic peptide
  Mass spectrometry peptide No. 288
LNELSFK

SEQ ID NO: 289 - protein, 8 aa, Artificial Sequence
  Description of Artificial Sequence: Synthetic peptide
  Mass spectrometry peptide No. 289
LNGESNLR

SEQ ID NO: 290 - protein, 5 aa, Artificial Sequence
  Description of Artificial Sequence: Synthetic peptide
  Mass spectrometry peptide No. 290
LNIPK

SEQ ID NO: 291 - protein, 15 aa, Artificial Sequence
  Description of Artificial Sequence: Synthetic peptide
  Mass spectrometry peptide No. 291
LNIPKLDFSSQADLR

SEQ ID NO: 292 - protein, 16 aa, Artificial Sequence
  Description of Artificial Sequence: Synthetic peptide
  Mass spectrometry peptide No. 292
LPQQANDYLNSFNWER

SEQ ID NO: 293 - protein, 12 aa, Artificial Sequence
  Description of Artificial Sequence: Synthetic peptide
  Mass spectrometry peptide No. 293
LPYTIITTPPLK

SEQ ID NO: 294 - protein, 14 aa, Artificial Sequence
  Description of Artificial Sequence: Synthetic peptide
  Mass spectrometry peptide No. 294
LQDFSDQLSDYYEK

SEQ ID NO: 295 - protein, 5 aa, Artificial Sequence
  Description of Artificial Sequence: Synthetic peptide
  Mass spectrometry peptide No. 295
LQQLK

SEQ ID NO: 296 - protein, 17 aa, Artificial Sequence
  Description of Artificial Sequence: Synthetic peptide
  Mass spectrometry peptide No. 296
LSLESLTSYFSIESSTK

SEQ ID NO: 297 - protein, 13 aa, Artificial Sequence
  Description of Artificial Sequence: Synthetic peptide
  Mass spectrometry peptide No. 297
LSNDMMGSYAEMK

SEQ ID NO: 298 - protein, 6 aa, Artificial Sequence
  Description of Artificial Sequence: Synthetic peptide
  Mass spectrometry peptide No. 298
LTALTK

SEQ ID NO: 299 - protein, 5 aa, Artificial Sequence
  Description of Artificial Sequence: Synthetic peptide
  Mass spectrometry peptide No. 299
LTIFK

SEQ ID NO: 300 - protein, 10 aa, Artificial Sequence
  Description of Artificial Sequence: Synthetic peptide
  Mass spectrometry peptide No. 300
LTISEQNIQR

SEQ ID NO: 301 - protein, 8 aa, Artificial Sequence
  Description of Artificial Sequence: Synthetic peptide
  Mass spectrometry peptide No. 301
LTLDIQNK

SEQ ID NO: 302 - protein, 10 aa, Artificial Sequence
  Description of Artificial Sequence: Synthetic peptide
  Mass spectrometry peptide No. 302
LVGFIDDAVK

SEQ ID NO: 303 - protein, 6 aa, Artificial Sequence
  Description of Artificial Sequence: Synthetic peptide
  Mass spectrometry peptide No. 303
LVTELR

SEQ ID NO: 304 - protein, 6 aa, Artificial Sequence
  Description of Artificial Sequence: Synthetic peptide
  Mass spectrometry peptide No. 304
LYSILK

SEQ ID NO: 305 - protein, 7 aa, Artificial Sequence
  Description of Artificial Sequence: Synthetic peptide
  Mass spectrometry peptide No. 305
MDMTFSK

SEQ ID NO: 306 - protein, 9 aa, Artificial Sequence
  Description of Artificial Sequence: Synthetic peptide
  Mass spectrometry peptide No. 306
MGLAFESTK

SEQ ID NO: 307 - protein, 4 aa, Artificial Sequence
  Description of Artificial Sequence: Synthetic peptide
  Mass spectrometry peptide No. 307
MNFK

SEQ ID NO: 308 - protein, 14 aa, Artificial Sequence
  Description of Artificial Sequence: Synthetic peptide
  Mass spectrometry peptide No. 308
MTSNFPVDLSDYPK

SEQ ID NO: 309 - protein, 12 aa, Artificial Sequence
  Description of Artificial Sequence: Synthetic peptide
  Mass spectrometry peptide No. 309
MYQMDIQQELQR

SEQ ID NO: 310 - protein, 11 aa, Artificial Sequence
  Description of Artificial Sequence: Synthetic peptide
  Mass spectrometry peptide No. 310
NAEWVYQGAIR

SEQ ID NO: 311 - protein, 7 aa, Artificial Sequence
  Description of Artificial Sequence: Synthetic peptide
  Mass spectrometry peptide No. 311
NFATSNK

SEQ ID NO: 312 - protein, 6 aa, Artificial Sequence
  Description of Artificial Sequence: Synthetic peptide
  Mass spectrometry peptide No. 312
NIFNFK

SEQ ID NO: 313 - protein, 9 aa, Artificial Sequence
  Description of Artificial Sequence: Synthetic peptide
  Mass spectrometry peptide No. 313
NIILPVYDK

SEQ ID NO: 314 - protein, 11 aa, Artificial Sequence
  Description of Artificial Sequence: Synthetic peptide
  Mass spectrometry peptide No. 314
NIQEYLSILTD

SEQ ID NO: 315 - protein, 15 aa, Artificial Sequence
  Description of Artificial Sequence: Synthetic peptide
  Mass spectrometry peptide No. 315
NIQEYLSILTDPDGK

SEQ ID NO: 316 - protein, 16 aa, Artificial Sequence
  Description of Artificial Sequence: Synthetic peptide
  Mass spectrometry peptide No. 316
NLEVKENFAGEATLQR

SEQ ID NO: 317 - protein, 18 aa, Artificial Sequence
  Description of Artificial Sequence: Synthetic peptide
  Mass spectrometry peptide No. 317
NLIGDFEVAEKINAFRAK

SEQ ID NO: 318 - protein, 15 aa, Artificial Sequence
  Description of Artificial Sequence: Synthetic peptide
  Mass spectrometry peptide No. 318
NLQNNAEWVYQGAIR

SEQ ID NO: 319 - protein, 16 aa, Artificial Sequence
  Description of Artificial Sequence: Synthetic peptide
  Mass spectrometry peptide No. 319
NLTDFAEQYSIQDWAK

SEQ ID NO: 320 - protein, 9 aa, Artificial Sequence
  Description of Artificial Sequence: Synthetic peptide
  Mass spectrometry peptide No. 320
NNALDFVTK

SEQ ID NO: 321 - protein, 9 aa, Artificial Sequence
  Description of Artificial Sequence: Synthetic peptide
  Mass spectrometry peptide No. 321
NNALDFVTK

SEQ ID NO: 322 - protein, 11 aa, Artificial Sequence
  Description of Artificial Sequence: Synthetic peptide
  Mass spectrometry peptide No. 322
NNQLTLDSNTK

SEQ ID NO: 323 - protein, 12 aa, Artificial Sequence
  Description of Artificial Sequence: Synthetic peptide
  Mass spectrometry peptide No. 323
NPNGYSFSIPVK

SEQ ID NO: 324 - protein, 21 aa, Artificial Sequence
  Description of Artificial Sequence: Synthetic peptide
  Mass spectrometry peptide No. 324
NQDVHSINLPFFETLQEYFER

SEQ ID NO: 325 - protein, 11 aa, Artificial Sequence
  Description of Artificial Sequence: Synthetic peptide
  Mass spectrometry peptide No. 325
NRNNALDFVTK

SEQ ID NO: 326 - protein, 11 aa, Artificial Sequence
  Description of Artificial Sequence: Synthetic peptide
  Mass spectrometry peptide No. 326
NRNNALDFVTK

SEQ ID NO: 327 - protein, 11 aa, Artificial Sequence
  Description of Artificial Sequence: Synthetic peptide
  Mass spectrometry peptide No. 327
NSEEFAAAMSR

SEQ ID NO: 328 - protein, 23 aa, Artificial Sequence
  Description of Artificial Sequence: Synthetic peptide
  Mass spectrometry peptide No. 328
NSLFFSAQPFEITASTNNEGNLK

SEQ ID NO: 329 - protein, 12 aa, Artificial Sequence
  Description of Artificial Sequence: Synthetic peptide
  Mass spectrometry peptide No. 329
NTFTLSCDGSLR

SEQ ID NO: 330 - protein, 12 aa, Artificial Sequence
  Description of Artificial Sequence: Synthetic peptide
  Mass spectrometry peptide No. 330
NTLELSNGVIVK

SEQ ID NO: 331 - protein, 17 aa, Artificial Sequence
  Description of Artificial Sequence: Synthetic peptide
  Mass spectrometry peptide No. 331
NYEAESSSGVPGTADSR

SEQ ID NO: 332 - protein, 6 aa, Artificial Sequence
  Description of Artificial Sequence: Synthetic peptide
  Mass spectrometry peptide No. 332
NYQLYK

SEQ ID NO: 333 - protein, 7 aa, Artificial Sequence
  Description of Artificial Sequence: Synthetic peptide
  Mass spectrometry peptide No. 333
PETEEAK

SEQ ID NO: 334 - protein, 7 aa, Artificial Sequence
  Description of Artificial Sequence: Synthetic peptide
  Mass spectrometry peptide No. 334
PLPFGGK

SEQ ID NO: 335 - protein, 8 aa, Artificial Sequence
  Description of Artificial Sequence: Synthetic peptide
  Mass spectrometry peptide No. 335
PPETEEAK

SEQ ID NO: 336 - protein, 7 aa, Artificial Sequence
  Description of Artificial Sequence: Synthetic peptide
  Mass spectrometry peptide No. 336
QAEAVLK

SEQ ID NO: 337 - protein, 8 aa, Artificial Sequence
  Description of Artificial Sequence: Synthetic peptide
  Mass spectrometry peptide No. 337
QELNGNTK

SEQ ID NO: 338 - protein, 10 aa, Artificial Sequence
  Description of Artificial Sequence: Synthetic peptide
  Mass spectrometry peptide No. 338
QGFFPDSVNK

SEQ ID NO: 339 - protein, 9 aa, Artificial Sequence
  Description of Artificial Sequence: Synthetic peptide
  Mass spectrometry peptide No. 339
QHIEAIDVR

SEQ ID NO: 340 - protein, 8 aa, Artificial Sequence
  Description of Artificial Sequence: Synthetic peptide
  Mass spectrometry peptide No. 340
QIDDIDVR

SEQ ID NO: 341 - protein, 6 aa, Artificial Sequence
  Description of Artificial Sequence: Synthetic peptide
  Mass spectrometry peptide No. 341
QNALLR

SEQ ID NO: 342 - protein, 8 aa, Artificial Sequence
  Description of Artificial Sequence: Synthetic peptide
  Mass spectrometry peptide No. 342
QSFDLSVK

SEQ ID NO: 343 - protein, 7 aa, Artificial Sequence
  Description of Artificial Sequence: Synthetic peptide
  Mass spectrometry peptide No. 343
QSWSVCK

SEQ ID NO: 344 - protein, 9 aa, Artificial Sequence
  Description of Artificial Sequence: Synthetic peptide
  Mass spectrometry peptide No. 344
QTEATMTFK

SEQ ID NO: 345 - protein, 12 aa, Artificial Sequence
  Description of Artificial Sequence: Synthetic peptide
  Mass spectrometry peptide No. 345
QTIIVVVENVQR

SEQ ID NO: 346 - protein, 18 aa, Artificial Sequence
  Description of Artificial Sequence: Synthetic peptide
  Mass spectrometry peptide No. 346
QVFLYPEKDEPTYILNIK

SEQ ID NO: 347 - protein, 10 aa, Artificial Sequence
  Description of Artificial Sequence: Synthetic peptide
  Mass spectrometry peptide No. 347
RESDEETQIK

SEQ ID NO: 348 - protein, 17 aa, Artificial Sequence
  Description of Artificial Sequence: Synthetic peptide
  Mass spectrometry peptide No. 348
RGIISALLVPPETEEAK

SEQ ID NO: 349 - protein, 13 aa, Artificial Sequence
  Description of Artificial Sequence: Synthetic peptide
  Mass spectrometry peptide No. 349
RMNFKQELNGNTK

SEQ ID NO: 350 - protein, 7 aa, Artificial Sequence
  Description of Artificial Sequence: Synthetic peptide
  Mass spectrometry peptide No. 350
SCDGSLR

SEQ ID NO: 351 - protein, 11 aa, Artificial Sequence
  Description of Artificial Sequence: Synthetic peptide
  Mass spectrometry peptide No. 351
SDGRVKYTLNK

SEQ ID NO: 352 - protein, 10 aa, Artificial Sequence
  Description of Artificial Sequence: Synthetic peptide
  Mass spectrometry peptide No. 352
SEEFAAAMSR

SEQ ID NO: 353 - protein, 11 aa, Artificial Sequence
  Description of Artificial Sequence: Synthetic peptide
  Mass spectrometry peptide No. 353
SEILAHWSPAK

SEQ ID NO: 354 - protein, 11 aa, Artificial Sequence
  Description of Artificial Sequence: Synthetic peptide
  Mass spectrometry peptide No. 354
SEYQADYESLR

SEQ ID NO: 355 - protein, 14 aa, Artificial Sequence
  Description of Artificial Sequence: Synthetic peptide
  Mass spectrometry peptide No. 355
SFDYHQFVDETNDK

SEQ ID NO: 356 - protein, 15 aa, Artificial Sequence
  Description of Artificial Sequence: Synthetic peptide
  Mass spectrometry peptide No. 356
SGSSTASWIQNVDTK

SEQ ID NO: 357 - protein, 7 aa, Artificial Sequence
  Description of Artificial Sequence: Synthetic peptide
  Mass spectrometry peptide No. 357
SHDELPR

SEQ ID NO: 358 - protein, 16 aa, Artificial Sequence
  Description of Artificial Sequence: Synthetic peptide
  Mass spectrometry peptide No. 358
SINLPFFETLQEYFER

SEQ ID NO: 359 - protein, 9 aa, Artificial Sequence
  Description of Artificial Sequence: Synthetic peptide
  Mass spectrometry peptide No. 359
SISAALEHK

SEQ ID NO: 360 - protein, 8 aa, Artificial Sequence
  Description of Artificial Sequence: Synthetic peptide
  Mass spectrometry peptide No. 360
SKEVPEAR

SEQ ID NO: 361 - protein, 12 aa, Artificial Sequence
  Description of Artificial Sequence: Synthetic peptide
  Mass spectrometry peptide No. 361
SKPTVSSSMEFK

SEQ ID NO: 362 - protein, 8 aa, Artificial Sequence
  Description of Artificial Sequence: Synthetic peptide
  Mass spectrometry peptide No. 362
SLHMYANR

SEQ ID NO: 363 - protein, 11 aa, Artificial Sequence
  Description of Artificial Sequence: Synthetic peptide
  Mass spectrometry peptide No. 363
SLVTTLNSDLK

SEQ ID NO: 364 - protein, 7 aa, Artificial Sequence
  Description of Artificial Sequence: Synthetic peptide
  Mass spectrometry peptide No. 364
SLWDFLK

SEQ ID NO: 365 - protein, 8 aa, Artificial Sequence
  Description of Artificial Sequence: Synthetic peptide
  Mass spectrometry peptide No. 365
SMYRELLK

SEQ ID NO: 366 - protein, 11 aa, Artificial Sequence
  Description of Artificial Sequence: Synthetic peptide
  Mass spectrometry peptide No. 366
SNTVASLHTEK

SEQ ID NO: 367 - protein, 10 aa, Artificial Sequence
  Description of Artificial Sequence: Synthetic peptide
  Mass spectrometry peptide No. 367
SPAFTDLHLR

SEQ ID NO: 368 - protein, 9 aa, Artificial Sequence
  Description of Artificial Sequence: Synthetic peptide
  Mass spectrometry peptide No. 368
SPSQADINK

SEQ ID NO: 369 - protein, 7 aa, Artificial Sequence
  Description of Artificial Sequence: Synthetic peptide
  Mass spectrometry peptide No. 369
SQAIATK

SEQ ID NO: 370 - protein, 10 aa, Artificial Sequence
  Description of Artificial Sequence: Synthetic peptide
  Mass spectrometry peptide No. 370
SSVIDALQYK

SEQ ID NO: 371 - protein, 9 aa, Artificial Sequence
  Description of Artificial Sequence: Synthetic peptide
  Mass spectrometry peptide No. 371
SVGFHLPSR

SEQ ID NO: 372 - protein, 19 aa, Artificial Sequence
  Description of Artificial Sequence: Synthetic peptide
  Mass spectrometry peptide No. 372
SVMAPFTMTIDAHTNGNGK

SEQ ID NO: 373 - protein, 17 aa, Artificial Sequence
  Description of Artificial Sequence: Synthetic
  Mass spectrometry peptide No. 373
SVSDGIAALDLNAVANK

SEQ ID NO: 374 - protein, 13 aa, Artificial Sequence
  Description of Artificial Sequence: Synthetic peptide
  Mass spectrometry peptide No. 374
SVSLPSLDPASAK

SEQ ID NO: 375 - protein, 18 aa, Artificial Sequence
  Description of Artificial Sequence: Synthetic peptide
  Mass spectrometry peptide No. 375
SYTLILPSLELPVLHVPR

SEQ ID NO: 376 - protein, 11 aa, Artificial Sequence
  Description of Artificial Sequence: Synthetic peptide
  Mass spectrometry peptide No. 376
TEVIPPLIENR

SEQ ID NO: 377 - protein, 8 aa, Artificial Sequence
  Description of Artificial Sequence: Synthetic peptide
  Mass spectrometry peptide No. 377
TFIEDVNK

SEQ ID NO: 378 - protein, 10 aa, Artificial Sequence
  Description of Artificial Sequence: Synthetic peptide
  Mass spectrometry peptide No. 378
TGISPLALIK

SEQ ID NO: 379 - protein, 20 aa, Artificial Sequence
  Description of Artificial Sequence: Synthetic peptide
  Mass spectrometry peptide No. 379
TIDQMQNSELQWPVPDIYLR

SEQ ID NO: 380 - protein, 13 aa, Artificial Sequence
  Description of Artificial Sequence: Synthetic peptide
  Mass spectrometry peptide No. 380
TIEMSAFGYVFPK

SEQ ID NO: 381 - protein, 18 aa, Artificial Sequence
  Description of Artificial Sequence: Synthetic peptide
  Mass spectrometry peptide No. 381
TILGTMPAFEVSLQALQK

SEQ ID NO: 382 - protein, 13 aa, Artificial Sequence
  Description of Artificial Sequence: Synthetic peptide
  Mass spectrometry peptide No. 382
TKNSEEFAAAMSR

SEQ ID NO: 383 - protein, 13 aa, Artificial Sequence
  Description of Artificial Sequence: Synthetic peptide
  Mass spectrometry peptide No. 383
TLADLTLLDSPIK

SEQ ID NO: 384 - protein, 6 aa, Artificial Sequence
  Description of Artificial Sequence: Synthetic peptide
  Mass spectrometry peptide No. 384
TLQELK

SEQ ID NO: 385 - protein, 14 aa, Artificial Sequence
  Description of Artificial Sequence: Synthetic peptide
  Mass spectrometry peptide No. 385
TLQGIPQMIGEVIR

SEQ ID NO: 386 - protein, 13 aa, Artificial Sequence
  Description of Artificial Sequence: Synthetic peptide
  Mass spectrometry peptide No. 386
TMTIDAHTNGNGK

SEQ ID NO: 387 - protein, 8 aa, Artificial Sequence
  Description of Artificial Sequence: Synthetic peptide
  Mass spectrometry peptide No. 387
TNGEHTSK

SEQ ID NO: 388 - protein, 7 aa, Artificial Sequence
  Description of Artificial Sequence: Synthetic peptide
  Mass spectrometry peptide No. 388
TPALHFK

SEQ ID NO: 389 - protein, 18 aa, Artificial Sequence
  Description of Artificial Sequence: Synthetic peptide
  Mass spectrometry peptide No. 389
TQFNNNEYSQDLDAYNTK

SEQ ID NO: 390 - protein, 7 aa, Artificial Sequence
  Description of Artificial Sequence: Synthetic peptide
  Mass spectrometry peptide No. 390
TSQCTLK

SEQ ID NO: 391 - protein, 14 aa, Artificial Sequence
  Description of Artificial Sequence: Synthetic peptide
  Mass spectrometry peptide No. 391
TSQCTLKEVYGFNP

SEQ ID NO: 392 - protein, 15 aa, Artificial Sequence
  Description of Artificial Sequence: Synthetic peptide
  Mass spectrometry peptide No. 392
TSSFALNLPTLPEVK

SEQ ID NO: 393 - protein, 6 aa, Artificial Sequence
  Description of Artificial Sequence: Synthetic peptide
  Mass spectrometry peptide No. 393
TTEVLR

SEQ ID NO: 394 - protein, 8 aa, Artificial Sequence
  Description of Artificial Sequence: Synthetic peptide
  Mass spectrometry peptide No. 394
VEDIPLAR

SEQ ID NO: 395 - protein, 7 aa, Artificial Sequence
  Description of Artificial Sequence: Synthetic peptide
  Mass spectrometry peptide No. 395
VHELIER

SEQ ID NO: 396 - protein, 17 aa, Artificial Sequence
  Description of Artificial Sequence: Synthetic peptide
  Mass spectrometry peptide No. 396
VIGNMGQTMEQLTPELK

SEQ ID NO: 397 - protein, 6 aa, Artificial Sequence
  Description of Artificial Sequence: Synthetic peptide
  Mass spectrometry peptide No. 397
VIGTLK

SEQ ID NO: 398 - protein, 5 aa, Artificial Sequence
  Description of Artificial Sequence: Synthetic peptide
  Mass spectrometry peptide No. 398
VLADK

SEQ ID NO: 399 - protein, 14 aa, Artificial Sequence
  Description of Artificial Sequence: Synthetic peptide
  Mass spectrometry peptide No. 399
VLLDQLGTTISFER

SEQ ID NO: 400 - protein, 10 aa, Artificial Sequence
  Description of Artificial Sequence: Synthetic peptide
  Mass spectrometry peptide No. 400
VLVDHFGYTK

SEQ ID NO: 401 - protein, 9 aa, Artificial Sequence
  Description of Artificial Sequence: Synthetic peptide
  Mass spectrometry peptide No. 401
VNDESTEGK

SEQ ID NO: 402 - protein, 16 aa, Artificial Sequence
  Description of Artificial Sequence: Synthetic peptide
  Mass spectrometry peptide No. 402
VNQNLVYESGSLNFSK

SEQ ID NO: 403 - protein, 16 aa, Artificial Sequence
  Description of Artificial Sequence: Synthetic peptide
  Mass spectrometry peptide No. 403
VNWEEEAASGLLTSLK

SEQ ID NO: 404 - protein, 21 aa, Artificial Sequence
  Description of Artificial Sequence: Synthetic peptide
  Mass spectrometry peptide No. 404
VNWEEEAASGLLTSLKDNVPK

SEQ ID NO: 405 - protein, 18 aa, Artificial Sequence
  Description of Artificial Sequence: Synthetic peptide
  Mass spectrometry peptide No. 405
VPLLLSEPINIIDALEMR

SEQ ID NO: 406 - protein, 20 aa, Artificial Sequence
  Description of Artificial Sequence: Synthetic peptide
  Mass spectrometry peptide No. 406
VPSYTLILPSLELPVLHVPR

SEQ ID NO: 407 - protein, 9 aa, Artificial Sequence
  Description of Artificial Sequence: Synthetic peptide
  Mass spectrometry peptide No. 407
VQGVEFSHR

SEQ ID NO: 408 - protein, 15 aa, Artificial Sequence
  Description of Artificial Sequence: Synthetic peptide
  Mass spectrometry peptide No. 408
VSALLTPAEQTGTWK

SEQ ID NO: 409 - protein, 7 aa, Artificial Sequence
  Description of Artificial Sequence: Synthetic peptide
  Mass spectrometry peptide No. 409
VSQEGLK

SEQ ID NO: 410 - protein, 7 aa, Artificial Sequence
  Description of Artificial Sequence: Synthetic peptide
  Mass spectrometry peptide No. 410
VSSFYAK

SEQ ID NO: 411 - protein, 9 aa, Artificial Sequence
  Description of Artificial Sequence: Synthetic peptide
  Mass spectrometry peptide No. 411
VSTAFVYTK

SEQ ID NO: 412 - protein, 12 aa, Artificial Sequence
  Description of Artificial Sequence: Synthetic peptide
  Mass spectrometry peptide No. 412
WKNEVRIHSGSF

SEQ ID NO: 413 - protein, 13 aa, Artificial Sequence
  Description of Artificial Sequence: Synthetic peptide
  Mass spectrometry peptide No. 413
WNFYYSPQSSPDK

SEQ ID NO: 414 - protein, 16 aa, Artificial Sequence
  Description of Artificial Sequence: Synthetic peptide
  Mass spectrometry peptide No. 414
YEAESSSGVPGTADSR

SEQ ID NO: 415 - protein, 9 aa, Artificial Sequence
  Description of Artificial Sequence: Synthetic peptide
  Mass spectrometry peptide No. 415
YENYELTLK

SEQ ID NO: 416 - protein, 12 aa, Artificial Sequence
  Description of Artificial Sequence: Synthetic peptide
  Mass spectrometry peptide No. 416
YGMVAQVTQTLK

SEQ ID NO: 417 - protein, 16 aa, Artificial Sequence
  Description of Artificial Sequence: Synthetic peptide
  Mass spectrometry peptide No. 417
YKLQDFSDQLSDYYEK

SEQ ID NO: 418 - protein, 11 aa, Artificial Sequence
  Description of Artificial Sequence: Synthetic peptide
  Mass spectrometry peptide No. 418
YNALDLTNNGK

SEQ ID NO: 419 - protein, 5 aa, Artificial Sequence
  Description of Artificial Sequence: Synthetic peptide
  Mass spectrometry peptide No. 419
YTLNK

SEQ ID NO: 420 - protein, 20 aa, Artificial Sequence
  Description of Artificial Sequence: Synthetic peptide
  Mass spectrometry peptide No. 420
YTYNYEAESSSGVPGTADSR

SEQ ID NO: 421 - protein, 7 aa, Artificial Sequence
  Description of Artificial Sequence: Synthetic peptide
  Mass spectrometry peptide No. 421
YYELEEK

(19) United States
(12) Patent Application Publication
(10) Pub. No.: US 2011/0190151 A1
(43) Pub. Date: Aug. 4, 2011

(54) METHODS OF DIAGNOSING CHRONIC CARDIAC ALLOGRAFT REJECTION

(76) Inventors: Bruce McManus, Vancouver (CA); Zsuzsanna Hollander, Vancouver (CA); David Lin, Richmond (CA); Robert Balshaw, Vancouver (CA); Robert McMaster, Vancouver (CA); Paul Keown, Delta (CA); Gabriela Cohen Freue, Vancouver (CA); Janet Wilson-McManus, Vancouver (CA); Raymond Ng, Vancouver (CA)

(21) Appl. No.: 12/937,221

(22) PCT Filed: Apr. 9, 2009
(86) PCT No.: PCT/CA2009/000514

§ 371 Date: Mar. 9, 2011

Related U.S. Application Data
(60) Provisional application No. 61/071,056, filed on Apr. 10, 2008.
(60) Provisional application No. 61/157,166, filed on Mar. 3, 2009.

(51) Int. Cl. C40B 30/04; C12Q 1/68; G01N 33/68; G01N 33/566

(52) U.S. Cl. 506/9; 435/6.1; 435/6.12; 435/6.11; 435/6.18; 436/501; 435/7.1; 435/7.92

(57) ABSTRACT

The present invention relates to methods of diagnosing chronic rejection of a cardiac allograft using genomic expression profiling, proteomic expression profiling, or a combination of genomic and proteomic expression profiling.

This application claims priority benefit of U.S. Provisional applications 61/071,056, filed Apr. 10, 2008; and U.S. 61/157,166, filed Mar. 3, 2009, both of which are herein incorporated by reference.

### FIELD OF INVENTION

The present invention relates to methods of diagnosing chronic rejection of a cardiac allograft using genomic expression profiling, proteomic expression profiling, or a combination of genomic and proteomic expression profiling.

### BACKGROUND OF THE INVENTION

Transplantation is considered the primary therapy for patients with end-stage vital organ failure. While the availability of immunosuppressants such as cyclosporine and Tacrolimus has improved allograft recipient survival and wellbeing, identification of rejection of the allograft as early and as accurately as possible, and effective monitoring and adjusting immunosuppressive medication doses is still of primary importance to the continuing survival of the allograft recipient.

Rejection of an allograft may be generally described as the result of recipient's immune response to nonself antigens expressed by the donor tissues. Acute rejection may occur within days or weeks of the transplant, while chronic rejection may be a slower process, occurring months or years following the transplant.

At present, invasive biopsies, such as endomyocardial, liver core, and renal fine-needle aspiration biopsies, are widely regarded as the gold standard for the surveillance and diagnosis of allograft rejections, but are invasive procedures which carry risks of their own (e.g. Mehra M R, et al. Curr. Opin. Cardiol. 2002 March; 17(2):131-136.). Biopsy results may also be subject to reproducibility and interpretation issues due to sampling errors and inter-observer variabilities, despite the availability of international guidelines such as the Banff schema for grading liver allograft rejection (Ormonde et al 1999. Liver Transplantation 5:261-268) or the Revised ISHLT transplantation scale (Stewart et al. 2005. J Heart Lung Transplant, 2005; 24: 1710-20). Although less invasive (imaging) techniques have been developed such as angiography and IVUS for monitoring chronic heart rejection, these alternatives are also susceptible to limitations similar to those associated with biopsies.

Development of cardiac allograft vasculopathy (CAV)) is widely recognized as a key limiting factor for the long term survival of cardiac transplant recipients and an indicator of chronic rejection of the allograft (CR). Current, the most commonly used standard for detection of CAV is coronary angiography, a procedure which is invasive and relatively insensitive. CAV is typically characterized by vascular injury and concentric fibrous intimal hyperplasia/vascular lesions along the lengths of affected coronary vessels in the heart allograft. As CAV is considered the major causes of death in patients who survive the first year after transplantation, early detection has become increasingly important. However, early diagnosis of CAV is often a difficult task, partly due by the lack of clinical symptoms for ischemia as a result of cardiac denervation. At present, coronary angiography is used as the standard diagnosis for CAV. Intravascular ultrasound (IVUS), a relatively more sensitive technique, albeit not as widely used in transplant centers, is another tool for the diagnosis of CAV (reviewed in Schmauss et al., 2008. Circulation 117:2131-2141). Life expectancy is also affected by the effects of chronic rejection—the long term (i.e. 10-year) survival rate of heart recipients is roughly 50%, and is largely limited by the development of cardiac allograft vasculopathy (CAV) as an expression of chronic rejection (CR).

The severity of acute allograft rejection as determined by biopsy may be graded to provide standardized reference indicia. The International Society for Heart and Lung Transplantation scale (ISHLT) provides a means of grading biopsy samples for heart transplant subjects (Table 1).

TABLE 1
International Society for Heart and Lung Transplantation grading of
acute heart transplant rejection for histopathologic biopsy analysis

| Grade | Comment |
|---|---|
| 0R | No acute cellular rejection: No evidence of mononuclear |
| | inflammation or myocyte damage or necrosis. |
| 1R | Mild, low-grade, acute cellular rejection: Mononuclear cells are |
| | present and there may be limited myocyte damage and necrosis. |
| 2R | Moderate, intermediate-grade, acute cellular rejection: Two or |
| | more foci of mononuclear cells with associated myocyte damage |
| | and necrosis are present. The damage may be found in the same |
| | biopsy, or two separate biopsies. Eosinophils may be present. |
| 3R | Severe, high-grade, acute cellular rejection: Widespread, diffuse |
| | myocyte damage and necrosis, and disruption of normal archi- |
| | tecture across several biopsies. Edema, interstitial hemorrhage |
| | and vasculitis may be present. The infiltrate may be |
| | polymorphous. |

Indicators of allograft rejection may include a heightened and localized immune response as indicated by one or more of localized or systemic inflammation, tissue injury, allograft infiltration of immune cells, altered composition and concentration of tissue- and blood-derived proteins, differential oxygenation of allograft tissue, edema, thickening of the endothelium, increased collagen content, altered intramyocardial blood flow, infection, necrosis of the allograft and/or surrounding tissue, and the like.

Allograft rejection may be described as ‘acute’ or ‘chronic’. Acute rejection is generally considered to be rejection of a tissue or organ allograft within ˜6 months of the subject receiving the allograft. Acute rejection may be characterized by cellular and humoral insults on the donor tissue, leading to rapid graft dysfunction and failure of the tissue or organ. Chronic rejection is generally considered to be rejection of a tissue or organ allograft beyond 6 months, and may be several years after receiving the allograft. Chronic rejection may be characterized by progressive tissue remodeling triggered by the alloimmune response and may lead to gradual neointimal formation within arteries, contributing to obliterative vasculopathy, parenchymal fibrosis and consequently, failure and loss of the graft. Generally, it is clinically assessed or diagnosed by IUVS (Intra Vascular Ultrasound), angiography and/or echocardiography, and may further include biopsy if deemed necessary (see, for example, Tsutsui et al 2001 Circulation 104:653-7; Kobashigawa et al 2005. J. American College of Cardiology 45:1532-7; Tuzcu et a12005. J American College of cardiology 45:1538-42). Depending on the nature and severity of the rejection, there may be overlap in the indicators or clinical variables observed in a subject undergoing, or suspected of undergoing, allograft rejection—either chronic or acute.

Attempts have been made to reduce the number of biopsies and invasive surveillance techniques per patient, but may be generally unsuccessful, due in part to the difficulty in pinpointing the sites where rejection starts or progresses, and also to the difficulty in assessing tissue without performing the actual biopsy. Noninvasive surveillance techniques have been investigated, and may provide a reasonable negative prediction of allograft rejection, but may be of less practical utility in a clinical setting (Mehra et al., supra).

Within the field of chronic allograft rejection, a myriad of markers are recited and apparently conflicting results may be presented in some cases. This conflict in the literature, added to the complexity of the genome (estimates range upwards of 30,000 transcriptional units), the variety of cell types (estimates range upwards of 200), organs and tissues, and expressed proteins or polypeptides (estimates range upwards of 80,000)) in the human body, renders the number of possible nucleic acid sequences, genes, proteins, metabolites or combinations thereof useful for diagnosing organ rejection is staggering. Variation between individuals presents additional obstacles, as well as the dynamic range of protein concentration in plasma (ranging from 10⁻⁶ to 10³ μg/mL, with many of the proteins present at very low concentrations) and an overwhelming quantities of the few, most abundant plasma proteins (constituting ˜99% of the total protein mass).

PCT Publications WO2006/083986, WO2006/122407, US Publications 2008/0153092, 2006/0141493, U.S. Pat. No. 7,026,121 and U.S. Pat. No. 7,235,358 disclose methods for using panels of biomarkers (proteomic or genomic) for diagnosing or detecting various disease states ranging from cancer to organ transplantation.

Borozdenkova et al. 2004 (J. Proteome Research 3:282-288) discloses that alpha B-crystallin and tropmyosin were elevated in a set of cardiac transplant subjects.

Roussoulieres et al., 2005 (Circulation 111:2636-44) discloses an implication of CHD5 in acute rejection in a mouse model of human heart transplantation.

Ishihara, 2008 (J. Mol Cell Cardiology 45:S33) discloses that ADIPOQ may have a role in cardiac transplantation, and Nakano (Transplant Immunology 2007 17:130-136) suggests that upregulation of ADIPOQ may be necessary for overcoming rejection in liver transplant subjects.

Hedman et al., 2007 (Pediatr Transplantation 11:481-490 discloses that a high APOB/APOA1 ratio is associated with angiographically detectable vasculopathy in pediatric cardiac allograft recipients, and that low HDL-C predicts the onset of transplant vasculpathy in these patient on pravastatin therapy.

Alterations in levels of IGFBP3, MST1, CDH5 have been observed in acute renal allograft rejection (Fukuda et al., 1998 Growth Horm IGF Res 8:481-6; Sarwal et al., 2003. New England J. Med 349:125-138; Roussoulieres et al., 2007 J. Biomed Biotechnol. doi:10.1155/2007/41705).

Matsui et al., 2003 (Physiol Genomics 15:199-208) disclose a gene expression profile of tolerizing allografts after costimulatory signal blockade in a murine cardiac transplant model.

A review by Fildes et al 2008 (Transplant Immunology 19:1-11) discusses the role of cell types in immune processes following lung transplantation, and discloses that AICL (CLEC2B) interaction with NK cell proteins may have a role in acute and chronic rejection.

Integration of multiple platforms (proteomics, genomics) has been suggested for diagnosis and monitoring of various cancers, however discordance between protein and mRNA expression is identified in the field (Chen et al., 2002. Mol Cell Proteomics 1:304-313; Nishizuka et al., 2003 Cancer Research 63:5243-5250). Previous studies have reported low correlations between genomic and proteomic data (Gygi S P et al. 1999. Mol Cell Biol. 19:1720-1730; Huber et al., 2004 Mol Cell Proteomics 3:43-55).

Methods of assessing or diagnosing allograft rejection, including chronic rejection, that are less invasive, repeatable and more robust (less susceptible to sampling and interpretation errors) are greatly desirable.

### SUMMARY OF THE INVENTION

The present invention relates to methods of diagnosing chronic rejection of a cardiac allograft using genomic expression profiling, proteomic expression profiling, or a combination of genomic and proteomic expression profiling,

The present invention relates to methods of diagnosing rejection, including chronic rejection, of a cardiac allograft using genomic or proteomic expression profiling.

In accordance with one aspect of the invention, there is provided a method of diagnosing chronic allograft rejection in a subject, the method comprising a) determining a genomic expression profile of one or more than one genomic markers in a biological sample from the subject, the genomic markers selected from the group comprising CHPT1, RPS26, GBP3, KLRC1/KLRC2, ZCCHC2, 242907_at, CLEC2B, PDK4, OSBP2, IFIT5; b) comparing the expression profile of the one or more than one genomic markers to a non-rejector profile; and c) determining whether the expression level of the one or more than one genomic markers is increased or decreased relative to the non-rejector profile, wherein the increase or decrease of the one or more than one genomic markers is indicative of the rejection status of the subject.

In accordance with another aspect of the invention, there is provided a kit for diagnosing chronic allograft rejection in a subject, the kit comprising reagents for specific and quantitative detection of one or more than one of CHPT1, RPS26, GBP3, KLRC1/KLRC2, ZCCHC2, 242907_at, CLEC2B, PDK4, OSBP2 or IFIT5 along with instructions for the use of such reagents and optionally, methods for analyzing the resulting data. The kit may further comprise one or more oligonucleotides for selective hybridization to one or more of a gene or transcript encoding some or part of CHPT1, RPS26, GBP3, KLRC1/KLRC2, ZCCHC2, 242907_at, CLEC2B, PDK4, OSBP2 or IFIT5. Instructions or other information useful to combine the kit results with those of other assays to provide a non-rejection cutoff index or control for the diagnosis of a subject's rejection status may also be provided in the kit.

In accordance with another aspect of the invention, CHPT1, RPS26, GBP3, KLRC1/KLRC2, ZCCHC2, 242907_at, CLEC2B, PDK4 and IFIT5 may be decreased relative to a control, and OSBP2 may be increased relative to a control.

In accordance with another aspect of the invention, the method further comprises obtaining a value for one or more clinical variables and comparing the one or more clinical variables to a control.

In accordance with another aspect of the invention, the control is an autologous control.

In accordance with another aspect of the invention, the method may further comprise determining the expression profile of one or more markers listed in Table 6.

In accordance with another aspect of the invention, the control is a non-rejection, allograft recipient subject or a non-allograft recipient subject.

In accordance with another aspect of the invention, the expression profile of the one or more than one genomic markers may be determined by detecting an RNA sequence corresponding to the one or more than one markers.

In accordance with another aspect of the invention, the genomic expression profile of the one or more than one genomic markers may be determined by PCR.

In accordance with another aspect of the invention, the genomic expression profile of the one or more than one genomic markers may be determined by hybridization.

In accordance with another aspect of the invention, the hybridization may be to an oligonucleotide.

In accordance with another aspect of the invention, the biological sample is a blood sample.

In accordance with another aspect of the invention, there is provided a method of a) determining proteomic expression profile of one or more than one proteomic markers in a biological sample from the subject, the one or more than one proteomic markers selected from the group comprising polypeptides encoded by IGFBP3, MST1, CDH5, C1QB, CFHR2, CPN1, APOB, HBB, GC and C9; b) comparing the expression profile of the one or more than one proteomic markers to a non-rejector profile; and c) determining whether an expression level of the one or more than one proteomics markers is increased or decreased relative to the non-rejector profile, wherein increase or decrease of the level of the one or more than one proteomic markers is indicative of the rejection status of the subject.

In accordance with another aspect of the invention, there is provided a kit for diagnosing chronic allograft rejection in a subject, the kit comprising reagents for specific and quantitative detection of one or more than one of the polypeptides encoded by CFHR2, CPN1, APOB, HBB, GC, C9, IGFBP3, MST1, CDH5 and C1QB along with instructions for the use of such reagents and methods for analyzing the resulting data. Instructions or other information useful to combine the kit results with those of other assays to provide a non-rejection cutoff index or control for the diagnosis of a subject's rejection status may also be provided in the kit.

In accordance with another aspect of the invention the level of polypeptides encoded by IGFBP3, MST1, CDH5 and C1QB may be decreased relative to a control, and CFHR2, CPN1, APOB, HBB, GC and C9 may be increased relative to a control.

In accordance with another aspect of the invention, the method further comprises obtaining a value for one or more clinical variables and comparing the one or more clinical variables to a control.

In accordance with another aspect of the invention, the control is an autologous control.

In accordance with another aspect of the invention the non-rejector profile is obtained from a non rejecting, allograft recipient subject or a non-allograft recipient subject.

In accordance with another aspect of the invention the proteomic expression profile may be determined by an immunologic assay.

In accordance with another aspect of the invention the proteomic expression profile may be determined by ELISA.

In accordance with another aspect of the invention the proteomic expression profile may be determined by mass spectrometry.

In accordance with another aspect of the invention the proteomic expression profile may be determined by an isotope or isobaric tagging method.

It is therefore an advantage of some aspects of the present invention to provide a method of diagnosing chronic allograft rejection without a biopsy of the transplanted tissue or organ.

The present invention also relates to methods of diagnosing chronic rejection of a cardiac allograft using genomic expression and proteomic expression profiling. In accordance with another aspect of the invention, there is provided a method of diagnosing allograft rejection in a subject, the method comprising: a) determining the genomic expression profile of one or more than one markers in a biological sample from the subject, the markers selected from the group comprising genomic marker OSBP2, CHPT1, RPS26, GBP3, KLRC1, ZCCHC2, 242907, CLEC2B, PDK4 and IFIT5; b) determining the proteomic expression profile of proteomic markers selected from the group comprising a polypeptide encoded by CFHR2, CPN1, APOB, HBB, GC, C9, IGFBP3, MST1, CDH5 and C1QB in the biological sample; c) comparing the genomic and proteomic expression profiles to a control profile; and d) determining whether the genomic or proteomic expression level of the one or more than one markers is increased or decreased relative to the control profile, wherein increase or decrease of the one or more than one markers is indicative of rejection status.

In accordance with another aspect of the invention, there is provided a method of determining the chronic allograft rejection status of a subject using a combined panel of genomic and proteomic markers, the method comprising: a) determining the genomic expression profile of CHPT1, GBP3, 242907_at and CLEC2B genomic markers in a biological sample from the subject; b) determining proteomic expression profile of proteomic markers selected from the group comprising a polypeptide encoded by CFHR2, CPN1, GC and C1QB in the biological sample; c) comparing the genomic and proteomic expression profile to a control profile; and d) determining whether the genomic or proteomic expression level of the genomic and proteomic markers is increased or decreased relative to the control profile, wherein an increase in genomic markers CLDC2B, CHPT1, 242907_at, GB3 and an increase in the polypeptides encoded by CFHR2, CPN1 and GC and a decrease in the polypeptide encoded by C1QB is indicative of the chronic rejection status of the subject.

In accordance with another aspect of the invention, the method further comprises obtaining a value for one or more clinical variables and comparing the one or more clinical variables to a control.

In accordance with another aspect of the invention, the control is a non-rejection, allograft recipient subject or a non-allograft recipient subject.

In accordance with another aspect of the invention, there is provided a kit for assessing, predicting or diagnosing chronic allograft rejection in a subject, the kit comprising reagents for specific and quantitative detection of one or more than one of comprising genomic markers OSBP2, CHPT1, RPS26, GBP3, KLRC1, ZCCHC2, 242907, CLEC2B, PDK4 and IFIT5, and proteomic markers comprising a polypeptide encoded by CFHR2, CPN1, APOB, HBB, GC, C9, IGFBP3, MST1, CDH5 and C1QB, along with instructions for the use of such reagents and optionally, methods for analyzing the resulting data. Instructions or other information useful to combine the kit results with those of other assays to provide a non-rejection cutoff index or control for the diagnosis of a subject's rejection status may also be provided in the kit.

It is therefore an advantage of some aspects of the present invention to provide a method of diagnosing chronic allograft rejection without a biopsy of the transplanted tissue or organ.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1. A biomarker panel of 10 genes is used to classify chronic rejection (n=7) (solid diamond) from stable subjects (n=6) (solid circle). The list of genes for this biomarker panel include: choline phosphotransferase 1, ribosomal protein S26, guanylate binding protein 3, killer cell lectin-like receptor subfamily C, member 1, zinc finger, CCHC domain containing 2, 242907_at, C-type lectin domain family 2, member B, pyruvate dehydrogenase kinase, isozyme 4, oxysterol binding protein 2, interferon-induced protein with tetratricopeptide repeats 5.

FIG. 2. The biomarker panel as identified in FIG. 1 was applied using LDA to 31 samples to evaluate the classification value of the panel. 83% of those with chronic rejection (solid line) as identified by the methods above were correctly classified. 91% of the stable subjects (stippled line) were classified correctly.

FIG. 3 shows a proposed relationship between the biomarkers NKG2C, NKGWa, PDK4 and CHPT1.

FIG. 4 shows a heatmap based on the 106 probe sets, corresponding to 106 genes, with FDR <10%.

FIG. 5 shows a heatmap based on the 14 differentially expressed protein groups (p-value <0.05). The protein group codes are listed along the right hand side of the heatmap. Chronic samples (grey bar)—leftmost seven columns (1-7); stable samples (black bar)—rightmost six columns (8-13).

FIG. 6 shows a Striplot based on the classification results of the 12 test cohort samples using genomic, proteomic and combinatorial biomarker panels. Values for linear discriminant (LD) variables for all three classifiers (‘HP4’, ‘H4’ and ‘Combinatorial” for the genomic, proteomic and combinatorial classifiers, respectively) have been re-centered to calibrate the cut-off lines for classification to zero. Centers of the LD variable values (or the classifier ‘score’) for CR (open star) and S (solid star) samples in the training set are shown. The solid circles and solid squares correspond to the LD variable/classifier score for each of the S and CR samples, respectively in the test cohort.

FIG. 7 A-T shows target sequences (SEQ ID NOs: 1-10, 37-46) of nucleic acid markers useful for diagnosis of chronic cardiac allograft rejection, listed in Table 6.

FIG. 8 A-R shows amino acid sequences (SEQ ID NOs: 11-12, 14-17, 21-23, 25, 27-28 and 31-36) of proteomic markers useful for diagnosis of chronic cardiac allograft rejection, listed in Table 8.

FIG. 9 shows exemplary peptides identified in iTRAQ assays according to some embodiments of the present invention. The list further includes their assigned protein group codes and SEQ ID NOs: 47-421.

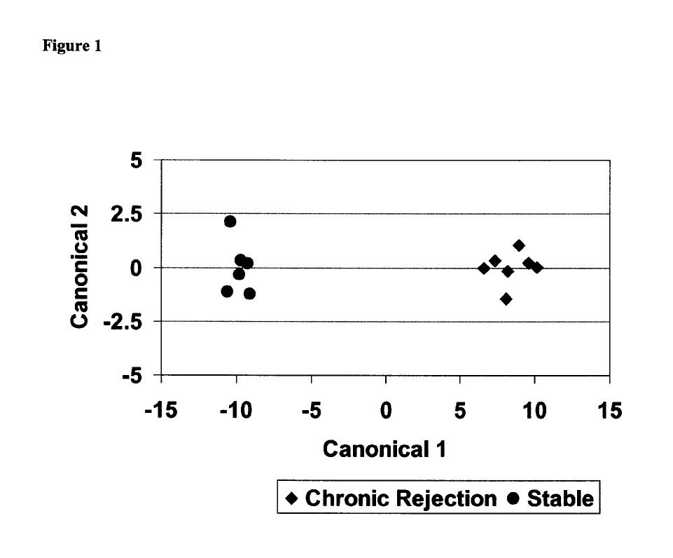

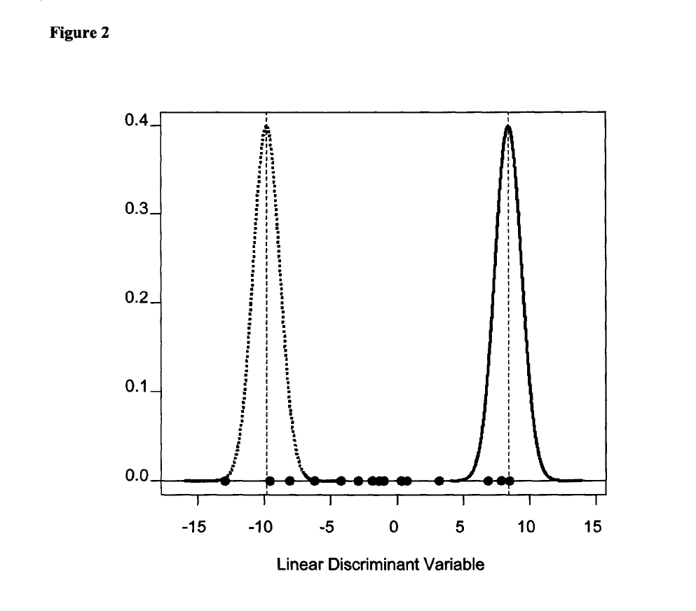

### DETAILED DESCRIPTION

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The present invention provides for methods of diagnosing rejection in a subject that has received a tissue or organ allograft, specifically a cardiac allograft.

The present invention provides genomic, nucleic acid, proteomic expression profiles or a combination of genomic and proteomic expression profiles related to the assessment, prediction or diagnosis of allow-aft rejection in a subject. While several of the elements in the genomic or proteomic expression profiles may be individually known in the existing art, the specific combination of the altered expression levels (increased or decreased relative to a control) of specific sets of genomic or proteomic markers comprise a novel combination useful for assessment, prediction or diagnosis or allograft rejection in a subject.

An allograft is an organ or tissue transplanted between two genetically different subjects of the same species. The subject receiving the allograft is the ‘recipient’, while the subject providing the allograft is the ‘donor’. A tissue or organ allograft may alternately be referred to as a ‘transplant’, a ‘graft’, an ‘allograft’, a ‘donor tissue’ or ‘donor organ’, or similar terms. A transplant between two subjects of different species is a xenograft.

Subjects may present with a variety of symptoms or clinical variables well-known in the literature, however none of these of itself is predictive or diagnostic of allograft rejection. A myriad of clinical variables may be used in assessing a subject having, or suspected of having, allograft rejection, in addition to biopsy of the allograft. The information obtained from these clinical variables is then used by a clinician, physician, veterinarian or other practitioner in a clinical field in attempts to determine if rejection is occurring, and how rapidly it progresses, to allow for modification of the immunosuppressive drug therapy of the subject. Examples of clinical variables are described in Table 2.

Clinical variables (optionally accompanied by biopsy), while currently the only practical tools available to a clinician in mainstream medical practice, are not always able to cleanly differentiate between an CR (chronic rejector) and an NR (non rejector, stable, or control) subject. While the extreme subjects may be correctly classified as CR or NR, the bulk of the subjects fall in the middle range and their status is unclear. This does not negate the value of the clinical variables in the assessment of allograft rejection, but instead indicates their limitation when used in the absence of other methods.

TABLE 2
Clinical variables for possible use in assessment of allograft rejection.

| | Renal/Heart/ | |
|---|---|---|
| Clinical Variable Name | Liver/All | Variable Explanation |
| Primary Diagnosis | All | Diagnosis leading to transplant |
| Secondary Diagnosis | All | Diagnosis leading to transplant |
| “Transplant Procedure - Living | | |
| related, Living unrelated, or | | |
| cadaveric” | | |
| Blood Type | All | Blood Type |
| Blood Rh | All | Blood Rh |
| Height (cm) | All | Height (cm) |
| Weight (kg) | All | Weight (kg) |
| BMI | All | Calculation: Weight/(Height)2 |
| Liver Ascites | All | |
| HLA A1 | All | |
| HLA A2 | All | |
| HLA B1 | All | |
| HLA B2 | All | |
| HLA DR1 | All | |
| HLA DR2 | All | |
| CMV | All | Viral Status |
| CMV Date | All | Date of viral status |
| HIV | All | Viral Status |
| HBV | All | Viral Status |
| HBV Date | All | Date of viral status |
| HbsAb | All | Viral Status |
| HbcAb (Total) | All | Viral Status |
| HBvDNA | All | Viral Status |
| HCV | All | Viral Status |
| HCV Genotype | All | Hepatitis C genotype |
| HCV Genotype Sub | All | “Hepatitis C genotype, subtype” |
| EBV | All | Viral Status |
| Zoster | All | Viral Status |
| Dialysis Start Date | All | Dialysis Start Date |
| Dialysis Type | All | Dialysis Type |
| Cytoxicity Current Level | All | |
| Cytoxicity Current Date | All | |
| Cytoxicity Peak Level | All | |
| Cytoxicity Peak Date | All | |
| Flush Soln | All | Type of Flush Solution used at transplant |
| Cold Time 1 | All | |
| Cold Time 2 | All | |
| Re-Warm Time 1 | All | |
| Re-Warm Time 2 | All | |
| HTLV 1 | All | |
| HTLV 2 | All | |
| HCV RNA | All | |
| 24 hr Urine | All | 24 Hour urine output |
| Systolic Blood Pressure | All | Blood Pressure reading |
| Diastolic Blood Pressure | All | Blood Pressure reading |
| 24 Hr Urine | All | 24 hour urine |
| Sodium | All | Blood test |
| Potassium | All | Blood test |
| Chloride | All | Blood test |
| Total CO2 | All | Blood test |
| Albumin | All | Blood test |
| Protein | All | Blood test |
| Calcium | All | Blood test |
| Inorganic Phosphate | All | Blood test |
| Magnesium | All | Blood test |
| Uric Acid | All | Blood test |
| Glucose | All | Blood test |
| Hemoglobin A1C | All | Blood test |
| CPK | All | Blood test |
| Parathyroid Hormone | All | Blood test |
| Homocysteine | All | Blood test |
| Urine Protein | All | Urine test |
| Creatinine | All | Blood test |
| BUN | All | Blood test |
| Hemaglobin | All | Blood test |
| Platelet Count | All | Blood test |
| WBC Count | All | Blood test |
| Prothrombin Time | All | Blood test |
| Partial Thromboplastin Time | All | Blood test |
| INR | All | Blood test |
| Gamma GT | All | Blood test |
| AST | All | Blood test |
| Alkaline Phosphatase | All | Blood test |
| Amylase | All | Blood test |
| Total Bilirubin | All | Blood test |
| Direct Bilirubin | All | Blood test |
| LDH | All | Blood test |
| ALT | All | Blood test |
| Triglycerides | All | Blood test |
| Cholesterol | All | Blood test |
| HDL Cholesterol | All | Blood test |
| LDL Cholesterol | All | Blood test |
| FEV1 | All | Lung function test |
| FVC | All | Lung function test |
| Total Ferritin | All | Blood test |
| TIBC | All | Blood test |
| Transferrin Saturated | All | Blood test |
| Ferritin | All | Blood test |
| Angiography | Heart | Heart function test |
| Intravascular ultrasound | Heart | Heart function test |
| Dobutamine Stress | Heart | Heart function test |
| Echocardiography | | |
| Cyclosporine WB | All | Immunosuppressive levels |
| Cyclosporine 2 hr | All | Immunosuppressive levels |
| Tacrolimus WB | All | Immunosuppressive levels |
| Sirolimus WB | All | Immunosuppressive total daily dose |
| Solumedrol | All | Immunosuppressive total daily dose |
| Prednisone | All | Immunosuppressive total daily dose |
| Prednisone ALT | All | Immunosuppressive total daily dose |
| Tacrolimus | All | Immunosuppressive total daily dose |
| Cyclosporine | All | Immunosuppressive total daily dose |
| Imuran | All | Immunosuppressive total daily dose |
| Mycophonelate Mofetil | All | Immunosuppressive total daily dose |
| Sirolimus | All | Immunosuppressive total daily dose |
| OKT3 | All | Immunosuppressive total daily dose |
| ATG | All | Immunosuppressive total daily dose |
| ALG | All | Immunosuppressive total daily dose |
| Basiliximab | All | Immunosuppressive total daily dose |
| Daclizumab | All | Immunosuppressive total daily dose |
| Ganciclovir | All | Anti-viral total daily dose |
| Lamivudine | All | Anti-viral total daily dose |
| Riboviron | All | Anti-viral total daily dose |
| Interferon | All | Anti-viral total daily dose |
| Hepatisis C Virus RNA | All | test for presence of HCV values ( ) |
| CMV Antigenemia | All | Antiviral and Virus |
| Valganciclovir | All | Anti-viral total daily dose |
| Neutrophil Number | All | Blood test |
| C Peptide | All | Blood test |
| Peg Interferon | All | Anti-viral total daily dose |
| GFR | All | Glomerular Filtration Rate |
| Complication Events | All | Complication Type |
| Biopsy Scores | Renal | (for acute rejection) |
| | | Borderline, 1A, 1B, 2A, 2B, 3, |
| | | Hyperacute |
| Biopsy Scores | Liver | (for acute rejection) |
| | | Portal inflammation, Bile duct |
| | | inflammation damage, Venous |
| | | endothelial inflammation each scored |
| | | from 1 to 3 |
| Donor Blood Type | All | Donor Blood Type |
| Donor Blood Rh | All | Donor Rh |
| Donor HLA A1 | All | Donor HLA A1 |
| Donor HLA A2 | All | Donor HLA A2 |
| Donor HLA B1 | All | Donor HLA B1 |
| Donor HLA B2 | All | Donor HLA B2 |
| Donor HLA DR1 | All | Donor HLA DR1 |
| Donor HLA DR2 | All | Donor HLA DR2 |
| Donor CMV | All | Donor CMV |
| Donor HIV | All | Donor HIV |
| Donor HBV | All | Donor HBV |
| Donor HbsAb | All | Donor HbsAb |
| Donor HbcAb (total) | All | Donor HbcAb (total) |
| Donor Hbdna | All | Donor Hbdna |
| Donor HCV | All | Donor HCV |
| Donor EBV | All | Donor EBV |

The multifactorial nature of allograft rejection prediction, diagnosis and assessment is considered in the art to exclude the possibility of a single biomarker that meets even one of the needs of prediction, diagnosis or assessment of allograft rejection. Strategies involving a plurality of markers may take into account this multifactorial nature. Alternately, a plurality of markers may be assessed in combination with clinical variables that are less invasive (e.g. a biopsy not required) to tailor the prediction, diagnosis and/or assessment of allograft rejection in a subject.

Regardless of the methods used for prediction, diagnosis and assessment of allograft rejection, earlier is better—from the viewpoint of preserving organ or tissue function and preventing more systemic detrimental effects. There is no ‘cure’ for allograft rejection, only maintenance of the subject at a suitably immunosuppressed state, or in some cases, replacement of the organ if rejection has progressed too rapidly or is too severe to correct with immunosuppressive drug intervention therapy.

Applying a plurality of mathematical and/or statistical analytical methods to a protein or polypeptide dataset, metabolite concentration data set, or nucleic acid expression dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers, uncertainty may be reduced, and clinically relevant core group of markers may be identified.

“Markers”, “biological markers” or “biomarkers” may be used interchangeably and refer generally to detectable (and in some cases quantifiable) molecules or compounds in a biological sample. A marker may be down-regulated (decreased), up-regulated (increased) or effectively unchanged in a subject following transplantation of an allograft. Markers may include nucleic acids (DNA or RNA), a gene, or a transcript, or a portion or fragment of a transcript in reference to ‘genomic’ markers (alternately referred to as “nucleic acid markers”); polypeptides, peptides, proteins or their precursors or isoforms, or fragments or portions thereof for ‘proteomic’ markers, or selected molecules, their precursors, intermediates or breakdown products (e.g. fatty acid, amino acid, sugars, hormones, or fragments or subunits thereof) (“metabolite markers” or “metabolomic markers”). A proteomic marker may be a polypeptide encoded by a gene. In some usages, these terms may reference the level or quantity of a particular protein, peptide, nucleic acid or polynucleotide, or metabolite (in absolute terms or relative to another sample or standard value) or the ratio between the levels of two proteins, polynucleotides, peptides or metabolites, in a subject's biological sample. The level may be expressed as a concentration, for example micrograms per milliliter; as a colorimetric intensity, for example 0.0 being transparent and 1.0 being opaque at a particular wavelength of light, with the experimental sample ranked accordingly and receiving a numerical score based on transmission or absorption of light at a particular wavelength; or as relevant for other means for quantifying a marker, such as are known in the art. In some examples, a ratio may be expressed as a unitless value. A “marker” may also reference to a ratio, or a net value following subtraction of a baseline value. A marker may also be represented as a ‘fold-change’, with or without an indicator of directionality (increase or decrease/up or down). The increase or decrease in expression of a marker may also be referred to as ‘down-regulation’ or ‘up-regulation’, or similar indicators of an increase or decrease in response to a stimulus, physiological event, or condition of the subject. A marker may be present in a first biological sample, and absent in a second biological sample; alternately the marker may be present in both, with a statistically significant difference between the two. Expression of the presence, absence or relative levels of a marker in a biological sample may be dependent on the nature of the assay used to quantify or assess the marker, and the manner of such expression will be familiar to those skilled in the art.

A marker may be described as being differentially expressed when the level of expression in a subject who is rejecting an allograft is significantly different from that of a subject or sample taken from a non-rejecting subject. A differentially expressed marker may be overexpressed or underexpressed as compared to the expression level of a normal or control sample.

A “profile” is a set of one or more markers and their presence, absence, relative level or abundance (relative to one or more controls). For example, a metabolite profile is a dataset of the presence, absence, relative level or abundance of metabolic markers. A proteomic profile is a dataset of the presence, absence, relative level or abundance of proteomic markers. A genomic or nucleic acid profile a dataset of the presence, absence, relative level or abundance of expressed nucleic acids (e.g. transcripts, mRNA, EST or the like). A profile may alternately be referred to as an expression profile.

The increase or decrease, or quantification of the markers in the biological sample may be determined by any of several methods known in the art for measuring the presence and/or relative abundance of a gene product or transcript. The level the markers may be determined as an absolute value, or relative to a baseline value, and the level of the subject's markers compared to a cutoff index (e.g. a non-rejection cutoff index). Alternately the relative abundance of the marker or markers may be determined relative to a control. The control may be a clinically normal subject (e.g. one who has not received an allograft) or may be an allograft recipient that has not previously demonstrated rejection.

In some embodiments, the control may be an autologous control, for example a sample or profile obtained from the subject before undergoing allograft transplantation. In some embodiments, the profile obtained at one or more time points (before, after or before and after transplantation) may be compared to one or more than one profiles obtained previously from the same subject. By repeatedly sampling the same biological sample from the same subject over time, a composite profile, illustrating marker level or expression over time may be provided. Sequential samples can also be obtained from the subject and a profile obtained for each, to allow the course of increase or decrease in one or more markers to be followed over time For example, an initial sample or samples may be taken before the transplantation, with subsequent samples being taken weekly, biweekly, monthly, bimonthly or at another suitable, interval and compared with profiles from samples taken previously. Samples may also be taken before, during and after administration of a course of a drug, for example an immunosuppressive drug.

Techniques, methods, tools, algorithms, reagents and other necessary aspects of assays that may be employed to detect and/or quantify a particular marker or set of markers are varied. Of significance is not so much the particular method used to detect the marker or set of markers, but what markers to detect. As is reflected in the literature, tremendous variation is possible. Once the marker or set of markers to be detected or quantified is identified, any of several techniques may be well suited, with the provision of appropriate reagents. One of skill in the art, when provided with the set of markers to be identified, will be capable of selecting the appropriate assay (for example, a PCR based or a microarray based assay for nucleic acid markers, an ELISA, protein or antibody microarray or similar immunologic assay, or in some examples, use of an iTRAQ, iCAT or SELDI proteomic mass spectrometric based method) for performing the methods disclosed herein.

The present invention provides nucleic acid expression profiles and proteomic expression profiles related to the assessment, prediction or diagnosis of allograft rejection in a subject. While several of the elements in the genomic or proteomic expression profiles may be individually known in the existing art, the specific combination of the altered expression levels (increased or decreased relative to a control) of specific sets of genomic or proteomic markers comprise a novel combination useful for assessment, prediction or diagnosis or allograft rejection in a subject.

For example, detection or determination, and in some cases quantification, of a nucleic acid may be accomplished by any one of a number methods or assays employing recombinant DNA technologies known in the art, including but not limited to, as sequence-specific hybridization, polymerase chain reaction (PCR), RT-PCR, microarrays and the like. Such assays may include sequence-specific hybridization, primer extension, or invasive cleavage. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of designing and selecting probes for use in microarrays or biochips, or for selecting or designing primers for use in PCR-based assays are known in the art. Once the marker or markers are identified and the sequence of the nucleic acid determined by, for example, querying a database comprising such sequences, or by having an appropriate sequence provided (for example, a sequence listing as provided herein), one of skill in the art will be able to use such information to select appropriate probes or primers and perform the selected assay.

Standard reference works setting forth the general principles of recombinant DNA technologies known to those of skill in the art include, for example: Ausubel et al, Current Protocols In Molecular Biology, John Wiley & Sons, New York (1998 and Supplements to 2001); Sambrook et al, Molecular Cloning: A Laboratory Manual, 2d Ed., Cold Spring Harbor Laboratory Press, Plainview, N.Y. (1989); Kaufman et al, Eds., Handbook Of Molecular And Cellular Methods In Biology And Medicine, CRC Press, Boca Raton (1995); McPherson, Ed., Directed Mutagenesis: A Practical Approach, IRL Press, Oxford (1991).

Proteins, protein complexes or proteomic markers may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids. Examples of methods employing mass spectrometry include those described in, for example, PCT Publication WO 2004/019000, WO 2000/00208, U.S. Pat. No. 6,670,194. Immunologic and non-immunologic methods may be combined to identify or characterize a protein or protein complex. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of producing antibodies for use in protein or antibody arrays, or other immunology based assays are known in the art. Once the marker or markers are identified and the amino acid sequence of the protein or polypeptide is identified, either by querying of a database or by having an appropriate sequence provided (for example, a sequence listing as provide herein), one of skill in the art will be able to use such information to prepare one or more appropriate antibodies and perform the selected assay.

For preparation of monoclonal antibodies directed towards a biomarker, any technique that provides for the production of antibody molecules by continuous cell lines in culture may be used. Such techniques include, but are not limited to, the hybridoma technique originally developed by Kohler and Milstein (1975, Nature 256:495-497), the trioma technique (Gustafsson et al., 1991, Hum. Antibodies Hybridomas 2:26-32), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). Human antibodies may be used and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). Techniques developed for the production of “chimeric antibodies” (Morrison et al, 1984, Proc. Natl. Acad. Sci. USA 81:6851-6855; Neuberger et al, 1984, Nature 312:604-608; Takeda et al, 1985, Nature 314:452-454) by splicing the genes from a mouse antibody molecule specific for a biomarker together with genes from a human antibody molecule of appropriate biological activity can be used; such antibodies are within the scope of this invention. Techniques described for the production of single chain antibodies (U.S. Pat. No. 4,946,778) can be adapted to produce a biomarker-specific antibodies. An additional embodiment of the invention utilizes the techniques described for) the construction of Fab expression libraries (Huse et al, 1989, Science 246:1275-1281) to allow rapid and easy identification of monoclonal Fab fragments with the desired specificity for a biomarker proteins. Non-human antibodies can be “humanized” by known methods (e.g., U.S. Pat. No. 5,225,539).

Antibody fragments that contain the idiotypes of a biomarker can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, are useful in the present invention

Standard reference works described herein and known to those skilled in the relevant art describe both immunologic and non-immunologic techniques, their suitability for particular sample types, antibodies, proteins or analyses. Standard reference works setting forth the general principles of immunology and assays employing immunologic methods known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993).

Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A subject's rejection status may be described as an “chronic rejector” (CR) or as a “non-rejector” (NR) or “stable” (S) and may be determined by comparison of the concentration of the markers to that of a non-rejector cutoff index. A “non-rejector cutoff index” is a numerical value or score, beyond or outside of which a subject is categorized as having a CR rejection status. The non-rejector cutoff index may be alternately referred to as a ‘control value’, a ‘control index’, or simply as a ‘control’. A non-rejector cutoff-index may be the concentration of individual markers in a control subject population and considered separately for each marker measured; alternately the non-rejector cutoff index may be a combination of the concentration of the markers, and compared to a combination of the concentration of the markers in the subject's sample provided for diagnosing. The control subject population may be a normal or healthy control population, or may be an allograft recipient population that has not, or is not, rejecting the allograft. The control may be a single subject, and for some embodiments, may be an autologous control. A control, or pool of controls, may be constant e.g. represented by a static value, or may be cumulative, in that the sample population used to obtain it may change from site to site, or over time and incorporate additional data points. For example, a central data repository, such as a centralized healthcare information system, may receive and store data obtained at various sites (hospitals, clinical laboratories or the like) and provide this cumulative data set for use with the methods of the invention at a single hospital, community clinic, for access by an end user (i.e. an individual medical practitioner, medical clinic or center, or the like).

The non-rejector cutoff index may be alternately referred to as a ‘control value’, a ‘control index’ or simply as a ‘control’. In some embodiments the cutoff index may be further characterized as being a metabolite cutoff index (for metabolite profiling of subjects), a genomic cutoff index (for genomic expression profiling of subjects), a proteomic cutoff index (for proteomic profiling of subjects), or the like.

A “biological sample” refers generally to body fluid or tissue or organ sample from a subject. For example, the biological sample may a body fluid such as blood, plasma, lymph fluid, serum, urine or saliva. A tissue or organ sample, such as a non-liquid tissue sample may be digested, extracted or otherwise rendered to a liquid form—examples of such tissues or organs include cultured cells, blood cells, skin, liver, heart, kidney, pancreas, islets of Langerhans, bone marrow, blood, blood vessels, heart valve, lung, intestine, bowel, spleen, bladder, penis, face, hand, bone, muscle, fat, cornea or the like. A plurality of biological samples may be collected at any one time. A biological sample or samples may be taken from a subject at any time, including before allograft transplantation, at the time of translation or at anytime following transplantation. A biological sample may comprise nucleic acid, such as deoxyribonucleic acid or ribonucleic acid, or a combination thereof, in either single or double-stranded form. When an organ is removed from a donor, the spleen of the donor or a part of it may be kept as a biological sample from which to obtain donor T-cells. When an organ is removed from a living donor, a blood sample may be taken, from which donor T-cells may be obtained. Alloreactive T-cells may be isolated by exploiting their specific interaction with antigens (including the MHC complexes) of the allograft. Methods to enable specific isolation of alloreactive T-cells are described in, for example PCT Publication WO 2005/05721, herein incorporated by reference.

A lymphocyte is nucleated or ‘white’ blood cell (leukocyte) of lymphoid stem cell origin. Lymphocytes include T-cells, B-cells natural killer cells and the like, and other immune regulatory cells. A “T-cell” is a class of lymphocyte responsible for cell-mediated immunity, and for stimulating B-cells. A stimulated B-cell produces antibodies for specific antigens. Both B-cells and T-cells function to recognize non-self antigens in a subject. Non-self antigens include those of viruses, bacteria and other infectious agents as well as allografts.

An alloreactive T-cell is a T-cell that is activated in response to an alloantigen. A T-cell that is reactive to a xenoantigen is a xenoreactive T-cell. A xenoantigen is an antigen from another species or species' tissue, such as a xenograft. Alloreactive T cells are the front-line of the graft rejection immune response. They are a subset (˜0.1-1%) of the peripheral blood mononuclear cells (PBMC) which recognize allogeneic antigens present on the foreign graft. They may infiltrate the foreign graft, to initiate a cascade of anti-graft immune response, which, if unchecked, will lead to rejection and failure of the graft. Alloreactive T cells, therefore provide specificity compared to other sources of markers, or may function as a complementary source of markers that differentiate between stages of organ rejection.

The term “subject” or “patient” generally refers to mammals and other animals including humans and other primates, companion animals, zoo, and farm animals, including, but not limited to, cats, dogs, rodents, rats, mice, hamsters, rabbits, horses, cows, sheep, pigs, goats, poultry, etc. A subject includes one who is to be tested, or has been tested for prediction, assessment or diagnosis of allograft rejection. The subject may have been previously assessed or diagnosed using other methods, such as those described herein or those in current clinical practice, or may be selected as part of a general population (a control subject).

A fold-change of a marker in a subject, relative to a control may be at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more, or any amount therebetween. The fold change may represent a decrease, or an increase, compared to the control value.

One or more than one includes 1, 2, 3, 4, 5, 6, 7, 8, 9, 10, 11, 12, 13, 14, 15, 16 or more.

“Down-regulation” or ‘down-regulated may be used interchangeably and refer to a decrease in the level of a marker, such as a gene, nucleic acid, metabolite, transcript, protein or polypeptide. “Up-regulation” or “up-regulated” may be used interchangeably and refer to an increase in the level of a marker, such as a gene, nucleic acid, metabolite, transcript, protein or polypeptide. Also, a pathway, such as a signal transduction or metabolic pathway may be up- or down-regulated.

Once a subject is identified as a chronic rejector, or at risk for becoming a chronic rejector by any method (genomic, proteomic or a combination thereof), therapeutic measures may be implemented to alter the subject's immune response to the allograft. The subject may undergo additional monitoring of clinical values more frequently, or using more sensitive monitoring methods. Additionally the subject may be administered immunosuppressive medicaments to decrease or increase the subject's immune response. Even though a subject's immune response needs to be suppressed to prevent rejection of the allograft, a suitable level of immune function is also needed to protect against opportunistic infection. Various medicaments that may be administered to a subject are known; see for example, Goodman and Gilman's The Pharmacological Basis of Therapeutics 11^{th} edition. Ch 52, pp 1405-1431 and references therein; L L Brunton, J S Lazo, K L Parker editors. Standard reference works setting forth the general principles of medical physiology and pharmacology known to those of skill in the art include: Fauci et al., Eds., Harrison's Principles Of Internal Medicine, 14th Ed., McGraw-Hill Companies, Inc. (1998). Other preventative and therapeutic strategies are reviewed in the medical literature—see, for example Kobashigawa et al. 2006. Nature Clinical Practice. Cardiovascular Medicine 3:203-21.

Therefore, the invention further provides for a method of predicting, assessing or diagnosing allograft rejection in a subject as provided by the present invention comprises 1) measuring the increase or decrease of one or more than one markers selected from the group comprising CHPT1, RPS26, GBP3, KLRC1/KLRC2, ZCCHC2, 242907_at, CLEC2B, PDK4, OSBP2, IFIT5; and 2) determining the ‘rejection status’ of the subject, wherein the determination of ‘rejection status’ of the subject is based on comparison of the subject's marker expression profile to a control marker expression profile.

Genomic Nucleic Acid Expression Profiling

A method of diagnosing chronic allograft rejection in a subject as provided by the present invention comprises 1) determining the expression profile of one or more than one markers in a biological sample from the subject, the markers selected from the group comprising CHPT1, RPS26, GBP3, KLRC1/KLRC2, ZCCHC2, 242907_at, CLEC2B, PDK4, OSBP2, IFIT5; 2) comparing the expression profile of the one or more than one markers to a non-rejector control profile; and 3) determining whether the expression level of the one or more than one markers is up-regulated or down-regulated relative to the control profile, wherein up-regulation or down-regulation of the one or more than one markers is indicative of the rejection status.

Using genomics methodologies, 106 genes which identified which were differentially expressed (FDR <10%) between 7 chronic rejection (CR) and 6 non-chronic rejection/stable (S) samples. 10 of these genes were further identified, based on a more stringent statistical cut-off (FDR <5% and fold-change >2), as the biomarker panel. Internal validation of this genomic biomarker panel using Linear Discriminant Analysis demonstrated that the 10 genes together, was able to classify 12 new ‘test’ samples with 83% sensitivity and specificity.

The phrase “gene expression data”, “gene expression profile” or “marker expression profile” as used herein refers to information regarding the relative or absolute level of expression of a gene or set of genes in a biological sample. The level of expression of a gene may be determined based on the level of RNA, such as mRNA, encoded by the gene. Alternatively, the level of expression may be determined based on the level of a polypeptide or fragment thereof encoded by the gene.

A ‘polynucleotide’, ‘oligonucleotide’ or ‘nucleotide polymer’ as used herein may include synthetic or mixed polymers of nucleic acids, including RNA, DNA or both RNA and DNA, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

An oligonucleotide includes variable length nucleic acids, which may be useful as probes, primers and in the manufacture of microarrays (arrays) for the detection and/or amplification of specific nucleic acids. Oligonucleotides may comprise DNA, RNA, PNA or other polynucleotide moieties as described in, for example, U.S. Pat. No. 5,948,902. Such DNA, RNA or oligonucleotide strands may be synthesized by the sequential addition (5′-3′ or 3′-5′) of activated monomers to a growing chain which may be linked to an insoluble support. Numerous methods are known in the art for synthesizing oligonucleotides for subsequent individual use or as a part of the insoluble support, for example in arrays (Bernfield M R. and Rottman F M. J. Biol. Chem. (1967) 242(18):4134-43; Sulston J. et al. PNAS (1968) 60(2):409-415; Gillam S. et al. Nucleic Acid Res. (1975) 2(5):613-624; Bonora G M. et al. Nucleic Acid Res. (1990) 18(11):3155-9; Lashkari D A. et al. PNAS (1995) 92(17):7912-5; McGall G. et al. PNAS (1996) 93(24):13555-60; Alber T J. et al. Nucleic Acid Res. (2003) 31(7):e35; Gao X. et al. Biopolymers (2004) 73(5):579-96; and Moorcroft M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or splicing of introns, for example, or may as yet to have any function attributed to them beyond the occurrence of the mutation of interest. A gene may also include one or more promoters, enhancers, transcription factor binding sites, termination signals or other regulatory elements. A gene or a transcript, may comprise nucleic acid.

The term “microarray,” “array,” or “chip” refers to a plurality of defined nucleic acid probes coupled to the surface of a substrate in defined locations. The substrate may be preferably solid. Microarrays have been generally described in the art in, for example, U.S. Pat. Nos. 5,143,854 (Pirrung), 5,424,186 (Fodor), 5,445,934 (Fodor), 5,677,195 (Winkler), 5,744,305 (Fodor), 5,800,992 (Fodor), 6,040,193 (Winkler), and Fodor et al. 1991. Science, 251:767-777. Each of these references is incorporated by reference herein in their entirety.

“Hybridization” includes a reaction in which one or more polynucleotides and/or oligonucleotides interact in an ordered manner (sequence-specific) to form a complex that is stabilized by hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York.), herein incorporated by reference.

Hybridization reactions can be performed under conditions of different “stringency”. The stringency of a hybridization reaction includes the difficulty with which any two nucleic acid molecules will hybridize to one another. Stringency may be increased, for example, by increasing the temperature at which hybridization occurs, by decreasing the ionic concentration at which hybridization occurs, or a combination thereof. Under stringent conditions, nucleic acid molecules at least 60%, 65%, 70%, 75% or more identical to each other remain hybridized to each other, whereas molecules with low percent identity cannot remain hybridized. An example of stringent hybridization conditions are hybridization in 6× sodium chloride/sodium citrate (SSC) at about 44-45° C., followed by one or more washes in 0.2×SSC, 0.1% SDS at 50° C., 55° C., 60° C., 65° C., or at a temperature therebetween.

Hybridization between two nucleic acids may occur in an antiparallel configuration—this is referred to as ‘annealing’, and the paired nucleic acids are described as complementary. A double-stranded polynucleotide may be “complementary”, if hybridization can occur between one of the strands of the first polynucleotide and the second. The degree of which one polynucleotide is complementary with another is referred to as homology, and is quantifiable in terms of the proportion of bases in opposing strands that are expected to hydrogen bond with each other, according to generally accepted base-pairing rules.

In general, sequence-specific hybridization involves a hybridization probe, which is capable of specifically hybridizing to a defined sequence. Such probes may be designed to differentiate between sequences varying in only one or a few nucleotides, thus providing a high degree of specificity. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Probes used in hybridization may include double-stranded DNA, single-stranded DNA and RNA oligonucleotides, and peptide nucleic acids. Hybridization conditions and methods for identifying markers that hybridize to a specific probe are described in the art—see, for example, Brown, T. “Hybridization Analysis of DNA Blots” in Current Protocols in Molecular Biology. F M Ausubel et al, editors. Wiley & Sons, 2003. doi: 10.1002/0471142727.mb0210s21. Suitable hybridization probes for use in accordance with the invention include oligonucleotides, polynucleotides or modified nucleic acids from about 10 to about 400 nucleotides, alternatively from about 20 to about 200 nucleotides, or from about 30 to about 100 nucleotides in length.

Specific sequences may be identified by hybridization with a primer or a probe, and this hybridization subsequently detected.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in Beverly, S M. Enzymatic Amplification of RNA by PCR (RT-PCR) in Current Protocols in Molecular Biology. F M Ausubel et al, editors. Wiley & Sons, 2003. doi: 10.1002/0471142727.mb1505s56. Synthesis of the replicate copies may include incorporation of a nucleotide having a label or tag, for example, a fluorescent molecule, biotin, or a radioactive molecule. The replicate copies may subsequently be detected via these tags, using conventional methods.

A primer may also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

A “probe set” (or ‘primer set’) as used herein refers to a group of oligonucleotides that may be used to detect one or more expressed nucleic acids or expressed genes. Detection may be, for example, through amplification as in PCR and RT-PCR, or through hybridization, as on a microarray, or through selective destruction and protection, as in assays based on the selective enzymatic degradation of single or double stranded nucleic acids. Probes in a probe set may be labeled with one or more fluorescent, radioactive or other detectable moieties (including enzymes). Probes may be any size so long as the probe is sufficiently large to selectively detect the desired gene—generally a size range from about 15 to about 25, or to about 30 nucleotides is of sufficient size. A probe set may be in solution, e.g. for use in multiplex PCR. Alternately, a probe set may be adhered to a solid surface, as in an array or microarray.

In some embodiments of the invention, a probe set for detection of nucleic acids expressed by a set of genomic markers comprising one or more CHPT1, RPS26, GBP3, KLRC1/KLRC2, ZCCHC2, 242907_at, CLEC2B, PDK4, OSBP2, IFIT5 is provided. Such a probe set may be useful for determining the rejection status of a subject. The probe set may comprise one or more pairs of primers for specific amplification (e.g. PCR or RT-PCR) of nucleic acid sequences corresponding to one or more of CHPT1, RPS26, GBP3, KLRC1/KLRC2, ZCCHC2, 242907_at, CLEC2B, PDK4, OSBP2, IFIT5. In another embodiment of the invention, the probe set is part of a microarray.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput array (Bortolin S. et al. 2004 Clinical Chemistry 50: 2028-36). This method amplifies genomic DNA by PCR followed by sequence-specific primer extension with universally tagged primers. The products are then sorted on a Tag-It array and detected using the Luminex xMAP system.

It will be appreciated by a person of skill in the art that any numerical designations of nucleotides within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular nucleotides at and around a mutational site. For example, the sequences represented by accession numbers CH471094.1, AC007068.17, AC91814.10, AY142147.1, BC005254.1, AB015628.1, AL550908.3, BG503026.1, BG540007.1, BG779377.1, X96719.1, DQ892509.2, DQ895723.2 all represent human CLEC2B nucleotide sequences, but may have some sequence differences, and numbering differences between them. As another example, the sequences represented by accession numbers NP_{—}005118.2, EAW96127.1, BAA76495.1, BAG36638.1, CAA65480.1, Q92478.2 all represent human CLEC2B polypeptide sequences, but may have some sequence differences, and numbering differences, between them.

Selection and/or design of probes, primers or probe sets for specific detection of expression of any gene of interest, including any of the above genes is within the ability of one of skill in the relevant art, when provided with one or more nucleic acid sequences of the gene of interest. Further, any of several probes, primers or probe sets, or a plurality of probes, primers or probe sets may be used to detect a gene of interest, for example, an array may include multiple probes for a single gene transcript—the aspects of the invention as described herein are not limited to any specific probes exemplified.

Sequence identity or sequence similarity may be determined using a nucleotide sequence comparison program (for DNA or RNA sequences, or fragments or portions thereof) or an amino acid sequence comparison program (for protein, polypeptide or peptide sequences, or fragments or portions thereof), such as that provided within DNASIS (for example, but not limited to, using the following parameters: GAP penalty 5, # of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (e.g. GAP, BESTFIT, FASTA, and BLAST), or by manual alignment and visual inspection.

If a nucleic acid or gene, polypeptide or sequence of interest is identified and a portion or fragment of the sequence (or sequence of the gene polypeptide or the like) is provided, other sequences that are similar, or substantially similar, may be identified using the programs exemplified above. For example, when constructing a microarray or probe sequences, the sequence and location are known, such that if a microarray experiment identifies a ‘hit’ (the probe at a particular location hybridizes with one or more nucleic acids in a sample, the sequence of the probe will be known (either by the manufacturer or producer of the microarray, or from a database provided by the manufacturer—for example the NetAffx databases of Affymetrix, the manufacturer of the Human Genome U133 Plus 2.0 Array). If the identity of the sequence source is not provided, it may be determined by using the sequence of the probe in a sequence-based search of one or more databases. For peptide or peptide fragments identified by proteomics assays, for example iTRAQ, the sequence of the peptide or fragment may be used to query databases of amino acid sequences as described above. Examples of such a database include those maintained by the National Centre for Biotechnology Information, or those maintained by the European Bioinformatics Institute.

A protein or polypeptide, nucleic acid or fragment or portion thereof may be considered to be specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity. For example, regarding a protein with multiple isoforms (either resulting from, for example, separate genes or variant splicing of the nucleic acid transcript from the gene, or post translational processing), an isoform may be specifically identified when it is differentiated from other isoforms from the same or a different species, by specific detection of a structure, sequence or motif that is present on one isoform and is absent, or not detectable on one or more other isoforms.

Access to the methods of the invention may be provided to an end user by, for example, a clinical laboratory or other testing facility performing the individual marker tests—the biological samples are provided to the facility where the individual tests and analyses are performed and the predictive method applied; alternately, a medical practitioner may receive the marker values from a clinical laboratory and use a local implementation or an internet-based implementation to access the predictive methods of the invention.

Determination of statistical parameters such as multiples of the median, standard error, standard deviation and the like, as well as other statistical analyses as described herein are known and within the skill of one versed in the relevant art. Use of a particular coefficient, value or index is exemplary only and is not intended to constrain the limits of the various aspects of the invention as disclosed herein.

Interpretation of the large body of gene expression data obtained from, for example, microarray experiments, or complex RT-PCR experiments may be a formidable task, but is greatly facilitated through use of algorithms and statistical tools designed to organize the data in a way that highlights systematic features. Visualization tools are also of value to represent differential expression by, for example, varying intensity and hue of colour (Eisen et al. 1998. Proc Natl Acad Sci 95:14863-14868). The algorithm and statistical tools available have increased in sophistication with the increase in complexity of arrays and the resulting datasets, and with the increase in processing speed, computer memory, and the relative decrease in cost of these.

Mathematical and statistical analysis of gene expression profiles may accomplish several things—identification of groups of genes that demonstrate coordinate regulation in a pathway or a domain of a biological system, identification of similarities and differences between two or more biological samples, identification of features of a gene expression profile that differentiate between specific events or processes in a subject, or the like. This may include assessing the efficacy of a therapeutic regimen or a change in a therapeutic regimen, monitoring or detecting the development of a particular pathology, differentiating between two otherwise clinically similar (or almost identical) pathologies, or the like.

Clustering methods are known and have been applied to microarray datasets, for example, hierarchical clustering, self-organizing maps, k-means or deterministic annealing. (Eisen et al, 1998 Proc Natl Acad Sci USA 95:14863-14868; Tamayo, P., et al. 1999. Proc Natl Acad Sci USA 96:2907-2912; Tavazoie, S., et al. 1999. Nat Genet 22:281-285; Alon, U., et al. 1999. Proc Natl Acad Sci USA 96:6745-6750). Such methods may be useful to identify groups of genes in a gene expression profile that demonstrate coordinate regulation, and also useful for the identification of novel genes of otherwise unknown function that are likely to participate in the same pathway or system as the others demonstrating coordinate regulation.

The pattern of gene expression in a biological sample may also provide a distinctive and accessible molecular picture of its functional state and identity (DeRisi 1997; Cho 1998; Chu 1998; Holstege 1998; Spellman 1998). Two different samples that have related gene expression patterns are therefore likely to be biologically and functionally similar to one another, conversely two samples that demonstrate significant differences may not only be differentiated by the complex expression pattern displayed, but may indicate a diagnostic subset of gene products or transcripts that are indicative of a specific pathological state or other physiological condition, such as allograft rejection.

Applying a plurality of mathematical and/or statistical analytical methods to a microarray dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers to all, the uncertainty is reduced, and clinically relevant core group of markers is identified.

Genomic Expression Profiling Markers (“Genomic Markers”)

The present invention provides for a core group of markers useful for the assessment, prediction or diagnosis of allograft rejection, including chronic allograft rejection, comprising genomic markers CHPT1, RPS26, GBP3, KLRC1/KLRC2, ZCCHC2, 242907_at, CLEC2B, PDK4, OSBP2, IFIT5.

Of the 22 genes or transcripts (Table 6) that were detected, quantified and found to demonstrate a statistically significant fold change in the whole-blood CR samples relative to non-rejecting transplant (NR) controls for at least one of the three modified t-tests applied, 10 markers are in the union set (statistically significant for all three tests). The fold-change for each marker in the larger set of 22 was at least two-fold, and may represent an increase/up-regulation or decrease/down-regulation of the gene or transcript in question.

The choline phosphotransferase 1 (CPT, CPT1) gene encodes a product involved in lipid metabolism, and possibly regulation of cell growth. Nucleotide sequences of human CHPT1 are known (e.g. GenBank Accession No. BC020819, BC050429, NW_{—}001838061, and NW_{—}925395).

The C-type lectin domain family 2, member B (CLEC2B, CLECSF2) gene encodes a member of the C-type lectin/C-type lectin-like domain (CTL/CTLD) superfamily. Members of this family share a common protein fold and have diverse functions, such as cell adhesion, cell-cell signalling, glycoprotein turnover, and roles in inflammation and immune response. The encoded type 2 transmembrane protein may function as a cell activation antigen. Nucleotide sequences of human CLEC2B are known (e.g. GenBank Accession No. CH471094.1, AC007068.17, AC91814.10, AY142147.1, BC005254.1, AB015628.1, AL550908.3, BG503026.1, BG540007.1, BG779377.1, X96719.1, DQ892509.2, DQ895723.2).

RPS26 (LOC644166/LOC644191/LOC728937/) may encode a ribosomal protein similar to the 40S ribosomal 26 protein. Nucleotide sequences related to this locus are known (e.g. GenBank Accession no. XM001721435.1, AC000134.1)

The gene encoding guanylate binding protein 3 (GBP3, DKFZp686E0974, DKFZp686L15228, FLJ10961) encodes a member of the guanylate-binding protein family, and may have interact with a member of the germinal center kinase family. Nucleotide sequences of human GBP3 are known (e.g. GenBank Accession No. NW_{—}001838589, NW_{—}921795, and NM_{—}018284).

Genes of the KLRC1/KLRC2 (killer cell lectin-like receptor subfamily C, member 1/killer cell lectin-like receptor subfamily C, member 2) family encode products that are transmembrane proteins preferentially expressed in NK cells and may have a role Plays a role as a receptor for the recognition of MHC class I HLA-E molecules by NK cells and some cytotoxic T-cells. Nucleotide sequences of human KLRC1 are known (e.g. GenBank Accession No.: NM_{—}213658, NM_{—}213657, NM_{—}007328, NM_{—}002259, BC012550, NW_{—}001838052 and NW_{—}925328). Nucleotide sequences of human KLRC2 are known (e.g. GenBank Accession No.: NM_{—}002260, NW_{—}001838052, NW_{—}925328, BC112039, BC093644, and BC106069).

The gene ZCCHC2 (zinc finger, CCHC domain containing 2) is also known as FLJ20281; KIAA1744; MGC13269; DKFZp451A185. Nucleotide sequences of human ZCCHC2 are known (e.g. GenBank Accession No.: NM_{—}017742, NW_{—}001838469, NW_{—}927106, NT_{—}025028.13 and BC006340).

The gene for PDK4 (pyruvate dehydrogenase kinase, isozyme 4) is a member of the PDK/BCKDK protein kinase family and encodes a mitochondrial protein That inhibits the mitochondrial pyruvate dehydrogenase complex by phosphorylation of the E1_alpha subunit, thus contributing to the regulation of glucose metabolism. Nucleotide sequences of human PDK4 are known (e.g. GenBank Accession No.: NM_{—}002612, NW_{—}001839064, NT_{—}079595, NW_{—}923574, and BC040239).

OSBP2 (oxysterol binding protein 2)—the membrane-bound protein encoded by this gene contains a pleckstrin homology (PH) domain and an oxysterol-binding region. Nucleotide sequences of human OSBP2 are known (e.g. GenBank Accession No.: NM_{—}030758, NM_{—}002556, BC118914, and AF288742).

The gene product of IFIT5 (interferon-induced protein with tetratricopeptide repeats 5) may have a role in interferon-regulated signaling and/or growth. Nucleotide sequences of human IFIT5 are known (e.g. GenBank Accession No.: NM_{—}012420, BC025786, CR457031, NW_{—}001838005, NW_{—}924884, and NT_{—}030059).

The present invention provides gene expression profiles related to the assessment, prediction or diagnosis of allograft rejection in a subject. While several of the elements in the gene expression profiles may be individually known in the existing art, the specific combination of their altered expression levels (increased or decreased relative to a control comprise a novel combination useful for assessment, prediction or diagnosis or allograft rejection in a subject.

Once a subject is identified as a chronic rejector, or at risk for becoming an chronic rejector, therapeutic measures may be implemented to alter the subject's immune response to the allograft. The subject may undergo additional monitoring of clinical values more frequently, or using more sensitive monitoring methods. Additionally the subject may be administered immunosuppressive medicaments to decrease or increase the subject's immune response. Even though a subject's immune response needs to be suppressed to prevent rejection of the allograft, a suitable level of immune function is also needed to protect against opportunistic infection. Various medicaments that may be administered to a subject are known; see for example, Goodman and Gilman's The Pharmacological Basis of Therapeutics 11^{th} edition. Ch 52, pp 1405-1431 and references therein; L L Brunton, J S Lazo, K L Parker editors. Other preventative and therapeutic strategies are reviewed in the medical literature—see, for example Kobashigawa et al. 2006. Nature Clinical Practice. Cardiovascular Medicine 3:203-21.

Biological Pathways Associated with Biomarkers of the Invention

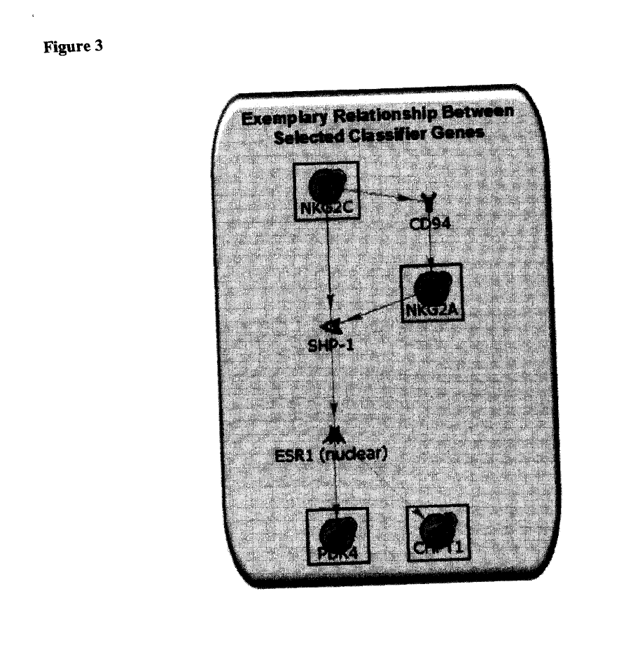

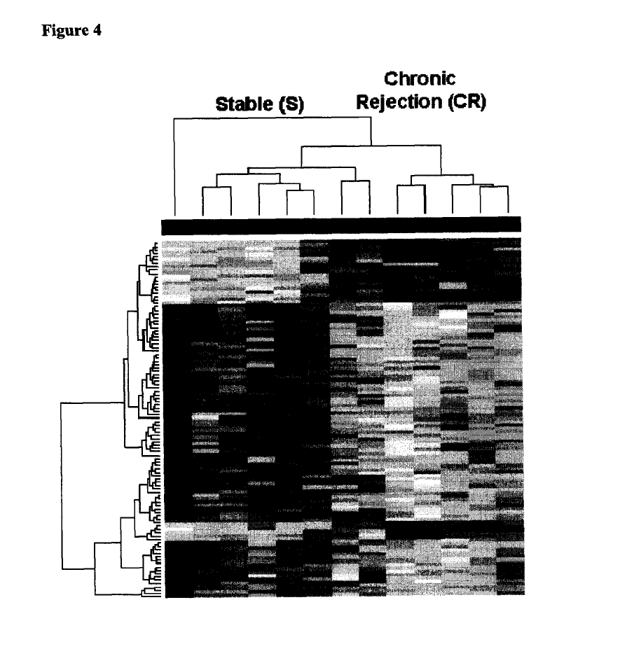

Biomarkers of the present invention are associated with biological pathways that may be particularly affected by the immune processes and a subject's response to an allograft rejection. Without wishing to be bound by theory, FIG. 3 illustrates a pathway-based relationship between the biomarkers KLRC2, KLRC1, PKD4 and CHPT1.

### 1. NKG2C (KLRC2)→CD94→NKG2A (KLRC1)

### 2. NKG2C/NKG2A (KLRC2/KLRC1)→SHP1→ESR1→PDK4 and CHPT1

### 3. ESR1→PDK4 and CHPT1

KLRC2, KLRC1, PKD4 and CHPT1 may, therefore have a biological role in the allograft rejection process, and represent therapeutic targets.

Without wishing to be bound by theory, HLA genes/polymorphism may have an impact on the outcome of transplantations (e.g. rejection, non rejection).

Large scale gene expression analysis methods, such as microarrays have indicated that groups of genes that have an interaction (often with two or more degrees of separation) are expressed together and may have common regulatory elements. Other examples of such coordinate regulation are known in the art, see, for example, the diauxic shift of yeast (DiRisi et al 1997 Science 278:680-686; Eisen et al. 1998. Proc Natl Acad Sci 95:14863-14868).

Without wishing to be bound by theory, other genes or transcript described herein, for example CHPT1, RPS26, GBP3, KLRC1/KLRC2, ZCCHC2, 242907_at, CLEC2B, PDK4, OSBP2 or IFIT5 may have a biological role in the allograft rejection process, and represent a therapeutic target.

The invention also provides for a kit for use in predicting or diagnosing a subject's rejection status. The kit may comprise reagents for specific and quantitative detection of one or more than one of CHPT1, RPS26, GBP3, KLRC1/KLRC2, ZCCHC2, 242907_at, CLEC2B, PDK4, OSBP2, IFIT5, along with instructions for the use of such reagents and methods for analyzing the resulting data. The kit may comprise reagents for specific and quantitative detection of one or more than one of CHPT1, RPS26, GBP3, KLRC1/KLRC2, ZCCHC2, 242907_at, CLEC2B, PDK4, OSBP2, IFIT5. The kit may be used alone for predicting or diagnosing a subject's rejection status, or it may be used in conjunction with other methods for determining clinical variables, or other assays that may be deemed appropriate. The kit may include, for example, a labelled oligonucleotide capable of selectively hybridizing to the marker. The kit may further include, for example, an oligonucleotide operable to amplify a region of the marker (e.g. by PCR). Instructions or other information useful to combine the kit results with those of other assays to provide a non-rejection cutoff index for the prediction or diagnosis of a subject's rejection status may also be provided. The kit may further include reagents for isolation of allo-reactive T-cells, and equipment or tools for isolation of the allo-reactive cells e.g.—magnetic beads, tubes for blood collection, buffers and the like, along with instructions for their use.

Alloreactive T-Cell Profiling

Profiling of the nucleic acids expressed in alloreactive lymphocytes, such as T-cells or T-lymphocytes (“alloreactive T-cell profiling”) may also be used for diagnosing allograft rejection. Alloreactive T-cell profiling may be used alone, or in combination with genomic expression profiling, proteomic profiling or metabolomic profiling.

Alloreactive T cells are the front-line of the graft rejection immune response. They are a subset (˜0.1-1%) of the peripheral blood mononuclear cells (PBMC) which recognize allogeneic antigens present on the foreign graft. They may infiltrate the foreign graft, to initiate a cascade of anti-graft immune response, which, if unchecked, will lead to rejection and failure of the graft. Alloreactive T cells, therefore, provide specificity compared to other sources of markers, or may function as a complementary source of markers that differentiate between stages of organ rejection. Gene expression profiles from an alloreactive T cell population may further be used across different organ transplants, and may also serve to better distinguish between organ rejection and immune activation due to other reasons (allergies, viral infection and the like).

Alloreactive T-cell profiling may also be used in combination with metabolite (“metabolomics”), genomic or proteomic profiling. Minor alterations in a subject's genome, such as a single base change or polymorphism, or expression of the genome (e.g. differential gene expression) may result in rapid response in the subject's small molecule metabolite profile. Small molecule metabolites may also be rapidly responsive to environmental alterations, with significant metabolite changes becoming evident within seconds to minutes of the environmental alteration—in contrast, protein or gene expression alterations may take hours or days to become evident. The list of clinical variables indicates several metabolites that may be used to monitor, for example, cardiovascular disease, obesity or metabolic syndrome—examples include cholesterol, homocysteine, glucose, uric acid, malondialdehyde and ketone bodies. Other non-limiting examples of small molecule metabolites are listed in Table 3.

Markers from alloreactive T-cells may be used alone for the diagnosis of allograft rejection, or may be used in combination with markers from whole blood.

Proteomic Profiling for Diagnosing Allograft Rejection

Proteomic profiling may also be used for diagnosing allograft rejection. Proteomic profiling may be used alone, or in combination with genomic expression profiling, metabolite profiling, or alloreactive T-cell profiling.

In some embodiments, the invention provides for a method of diagnosing or determining chronic allograft rejection in a subject comprising 1) determining the expression profile of one or more than one proteomic markers in a biological sample from the subject, the proteomic markers selected from the group comprising a polypeptide encoded by IGFBP3, MST1, CDH5, C1QB, CFHR2, CPN1, APOB, HBB, GC and C9; 2) comparing the expression profile of the one or more than one proteomic markers to a control profile; and 3) determining whether the expression level of the one or more than one proteomic markers is increased or decreased relative to the control profile, wherein increase or decrease of the one or more than one proteomic markers is indicative of the chronic rejection status.

The invention also provides for a method of predicting, assessing or diagnosing allograft rejection in a subject as provided by the present invention comprises 1) measuring the increase or decrease of one or more than one proteomic markers selected from the group comprising a polypeptide encoded by IGFBP3, MST1, CDH5, C1QB, CFHR2, CPN1, APOB, HBB, GC and C9; and 2) determining the chronic ‘rejection status’ of the subject, wherein the determination of ‘rejection status’ of the subject is based on comparison of the subject's proteomic marker expression profile to a control proteomic marker expression profile.

A myriad of methods for protein identification and quantitation are currently available, such as glycopeptide capture (Zhang et al., 2005. Mol Cell Proteomics 4:144-155), multidimensional protein identification technology (Mud-PIT) Washburn et al., 2001 Nature Biotechnology (19:242-247), and surface-enhanced laser desorption ionization (SELDI-TOF) (Hutches et al., 1993. Rapid Commun Mass Spec 7:576-580). Several isotope labelling methods which allow quantification of multiple protein samples, such as isobaric tags for relative and absolute protein quantification (iTRAQ) (Ross et al, 2004 Mol Cell Proteomics 3:1154-1169); isotope coded affinity tags (ICAT) (Gygi et al., 1999 Nature Biotechnology 17:994-999), isotope coded protein labelling (ICPL) (Schmidt et al., 2004. Proteomics 5:4-15), and N-terminal isotope tagging (NIT) (Fedjaev et al., 2007 Rapid Commun Mass Spectrom 21:2671-2679; Nam et al., 2005. J Chromatogr B Analyt Technol Biomed Life Sci. 826:91-107), have become increasingly popular due to their high-throughput performance, a trait particular useful in biomarker screening/identification studies.

Using proteomics methodologies, a combination of depletion of 14 most abundant proteins in plasma samples and sensitivity of iTRAQ-MALDI-TOF/TOF also proved to be another effective approach for large-scale screening and quantitative analysis of CR proteomic biomarkers. Briefly, subject plasma samples (control and allograft recipient) were depleted of the 14 most abundant proteins and quantitatively analyzed by iTRAQ-MALDI-TOF/TOF. This analysis resulted in the identification of 129 medium-to-low abundant protein (groups) which were detected in at least ⅔ of the CR and S samples. Of those, 14 had statistically significant, differential relative concentrations (p-value <0.5). 10 of the 14 candidates were further selected based on p-value cutoff at 0.03 as the “top” proteins. The 10 proteins, which together make up the proteomic biomarker panel, demonstrated a sensitivity and specificity of 83% in the internal validation process.

Thus, although single candidate biomarkers may not clearly differentiate groups (with some fold-changes being relatively small), together, the identified markers achieved a classification of about 83% sensitivity and about 83% specificity.

iTRAQ is one exemplary method used to detect, or determine the level of, the proteins, peptides, or fragments thereof that are proteomic markers of chronic allograft rejection. Other methods described herein, for example immunological based methods such as ELISA may also be useful for detecting, or determining the levels of, proteomic markers. Alternately, specific antibodies may be raised against the one or more proteins, isoforms, precursors, polypeptides, peptides, portions or fragments thereof, and the specific antibody used to detect the presence of the one or more proteomic marker in the sample. Methods of selecting suitable peptides, immunizing animals (e.g. mice, rabbits or the like) for the production of antisera and/or production and screening of hybridomas for production of monoclonal antibodies are known in the art, and described in the references disclosed herein.

Proteomic Expression Profiling Markers (“Proteomic Markers”)

One or more precursors, splice variants, isoforms may be encoded by a single gene Examples of genes and the isoforms, precursors and variants encoded are provided in Table 8, under the respective Protein Group Code (PGC).

A polypeptide encoded by CFHR2 (Complement factor H-related protein 2) includes a serum protein that are structurally and immunologically related to complement factor H. Nucleotide sequences encoding CFHR2 are known (e.g. GenBAnk Accession Nos. NM_{—}005666 BC022283.1, X64877.1 and BG566607.1). Amino acid sequences for a polypeptide encoded by CFHR2 (e.g. GenPept Accession Nos. P36980, CAA60375) are known.

A polypeptide encoded by CPN1 (Carboxypeptidase N catalytic chain precursor) includes a plasma metalloprotease that cleaves basic amino acids from the C terminus of peptides and proteins, and has a role in regulating the biologic activity of peptides such as kinins and anaphylatoxins. Nucleotide sequences encoding CPN1 are known (e.g. GenBank Accession Nos. NM_{—}001308 CR608830.1, X14329.1, AW950687.1). Amino acid sequences for a polypeptide encoded by CPN1 are known (e.g. GenPept Accession Nos. NP_{—}001073982, P22792, NP_{—}001295, NP_{—}001299, P15169).

A polypeptide encoded by APOB (Apolipoprotein B-100 (precursor), APOB100) includes an apolipoprotein of chylomicrons and low density lipoproteins (LDL) and is found in the plasma in 2 main forms, apoB48 and apoB100. Nucleic acid sequences encoding APOB are known (e.g. GenBank Accession Nos. NM_{—}019287, AK290844, NM_{—}000384). Amino acid sequences for a polypeptide encoded by APOB are known (e.g. GenPept Accession Nos. NP_{—}000375, P41238, AAB60718, I39470).

A polypeptide encoded by HBB (haemoglobin, beta locus, beta globin) plays a role in oxygen transport in the blood. Nucleotide sequences encoding HBB are known (e.g. GenBank Accession Nos. NM_{—}000518, NG_{—}000007, L48217.1). Amino acid sequences for a polypeptide encoded by HBB are known (e.g. GenPept Accession No. NP_{—}000509).

A polypeptide encoded by HBD (haemoglobin, delta locus) includes a constituent of hemoglobin. Nucleotide sequences encoding HBD are known (e.g. GenBank Accession Nos. AF339104.2, AY0.4468.1, BC069307.1, BC070282.1, BU664913.1). Amino acid sequences for a polypeptide encoded by HBD are known (e.g. GenPept Accession Nos. P02042.2, Q4F786, AAH70282.1).

A polypeptide encoded by GC (Group-specific component, DBP, VDBP, Vitamin D-binding protein) includes a serum protein in the albumin gene family, and has a role in binding and transporting vitamin D to target tissues. Nucleotide sequences encoding GC are known (e.g. GenBank Accession Nos. AK298433, NM_{—}000583, M12654.1 and BC022310.1). Amino acid sequences for a polypeptide encoded by GB are known (e.g. GenPept Accession No. NP_{—}000574, AAD14250, P02774).

A polypeptide encoded by C9 includes a complement component C9 precursor, which is the final component of the membrane attack complex (MAC) in the complement system Nucleic acid sequences encoding C9 are known (e.g. GenBank Accession Nos. NM_{—}001737, BC020721.1, CB157001.1, K02766.1 and CB135741.1.). Amino acid sequences for a polypeptide encoded by C9 are known (e.g. GenPept Accession Nos. NP_{—}001728, P02748)

A polypeptide encoded by IGFBP3 (insulin-like growth factor binding protein 3, IBP3) includes a carrier for IGF2 and IGF2 in the blood. Nucleic acid sequences encoding IGFBP3 are known (e.g. GenBank Accession Nos. NM_{—}000596, NM_{—}000598, NM_{—}001013398). Amino acid sequences for a polypeptide encoded by IGFBP3 are known (e.g. GenPept Accession Nos. P17936, NP_{—}001013416, NP_{—}000589, NP_{—}000587.

A polypeptide encoded by MST1 (macrophage stimulating 1, hepatocyte growth factor-like protein, HGFL) includes a polypeptide that regulates cell growth, cell motility and morphogenesis and has a role in embryonic organ development, adult organ regeneration and wound healing. Nucleic acid sequences encoding MST1 are known (e.g. GenBank Accession Nos. NM_{—}020998, DC315638.1, L11924.1, AK222893.1 and BM672747.1.). Amino acid sequences for a polypeptide encoded by MST1 are known (e.g. GenPept Accession Nos. NP_{—}066278, P26927).

A polypeptide encoded by CDH5 (cadherin-5, vascular endothelial cadherin, VE-cadherin) includes an endothelial adhesion molecule and may have a role in regulating endothelial function and vascular barrier integrity. Nucleic acid sequences encoding CDH5 are known (e.g. GenBank Accession Nos. NM_{—}001795, DC381809.1, X59796.1, U84722.1, AC132186.3 and X79981.1). Amino acid sequences for a polypeptide encoded by CDH5 are known (e.g. GenPept Accession Nos. NP_{—}001786, P33151).

A polypeptide encoded by C1QB (Complement component 1, q subcomponent, B chain) includes a polypeptide that is part of the first subcomponent C1q of the C1 protein of the complement system. Nucleic acid sequences encoding C1QB are known (e.g. GenBank Accession Nos. NG_{—}007283, NM_{—}000491). Amino acid sequences for a polypeptide encoded by C1QB are known (e.g GenPept Accession Nos. NP_{—}000482.3, P02746.2).

Combining Genomic and Proteomic Expression Profiling

A method of diagnosing chronic allograft rejection in a subject as provided by the present invention comprises 1) determining the expression profile of one or more than one markers in a biological sample from the subject, the markers selected from the group comprising genomic marker OSBP2, CHPT1, RPS26, GBP3, KLRC1, ZCCHC2, 242907, CLEC2B, PDK4 and IFIT5, and proteomic markers encoded by CFHR2, CPN1, APOB, HBB, GC, C9, IGFBP3, MST1, CDH5 and C1QB; 2) comparing the expression profile of the one or more than one to markers to a non-rejector profile; and 3) determining whether the expression level of the one or more than one markers is up-regulated or down-regulated relative to the control profile, wherein up-regulation or down-regulation of the one or more than one markers is indicative of the rejection status.

As described herein, robust statistical tests were applied to the genomic and proteomic platforms to identify differentially expressed genes and proteins. Using the candidates identified, a genomic, a proteomic, as well as combinatorial, biomarker panels were developed for discriminating between chronic rejection (CR) and non-chronic rejection/stable (S) samples.

A high-throughput approach and applied microarray plus qPCR, and multiplexed iTRAQ plus ELISA, was employed to identify potential whole blood genomic and plasma proteomic biomarkers of chronic rejection, respectively.

Interestingly, the genomic and proteomic biomarker panels identified in the current study had a similar level of performance in classifying CR and S samples. There does not appear to be an overlap between the identities of the 10 genes and 10 proteins (groups) across the panels. Unlike the proteomic platform which uses plasma samples, peripheral blood was used for the microarrays. Thus, the additional circulating components in the peripheral blood, such as red blood cells, platelets and especially white blood cells, may contribute to the differentially expressed genes detected. The impact of gene expressions from mononuclear cells (MNCs) and polymorphonuclear cells (PMNs) in the peripheral blood may also be significant, given that chronic inflammation is thought to play a major role in the development of CAV.

Gene ontology (GO)-based analyses revealed a greater degree of concordance between the genomic and proteomic panels of chronic cardiac allograft rejection. In general, the list of GO terms associated with each panel was independently unique, yet comparatively similar. At a high level (GO levels 3-5), biomarkers from the genomic and proteomic panels were shown, through enrichment analysis (p<0.05), to be involved in several similar biological and molecular processes. These processes include, but are not limited to: immune response, lipid transport, response to external stimulus and carbohydrate binding activities.

Given the sensitivity and specificity for the genomic and proteomic classifiers were similar, we also explored the possibility of a ‘combinatorial’ biomarker panel and tested its classification capability. Stepwise Discriminant Analysis (SDA) was applied separately to the genomic and proteomic biomarker panels to generate the best combination of candidates from each platform. The resulting biomarker panel incorporated 4 probe sets and 4 protein group codes (PGCs).

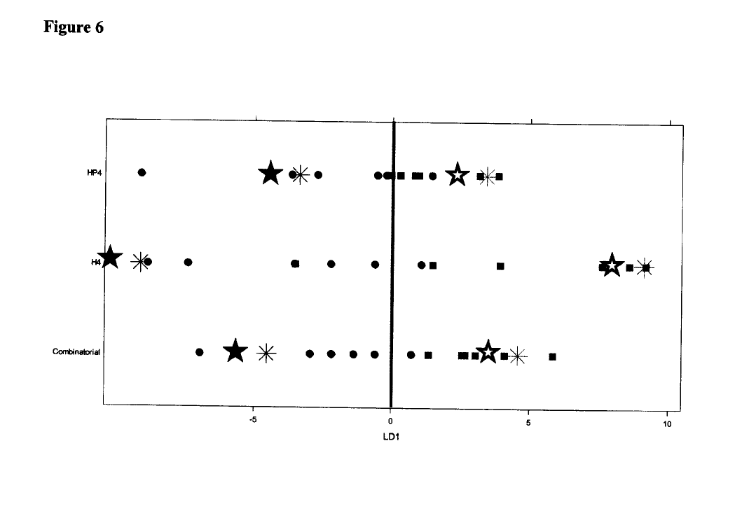

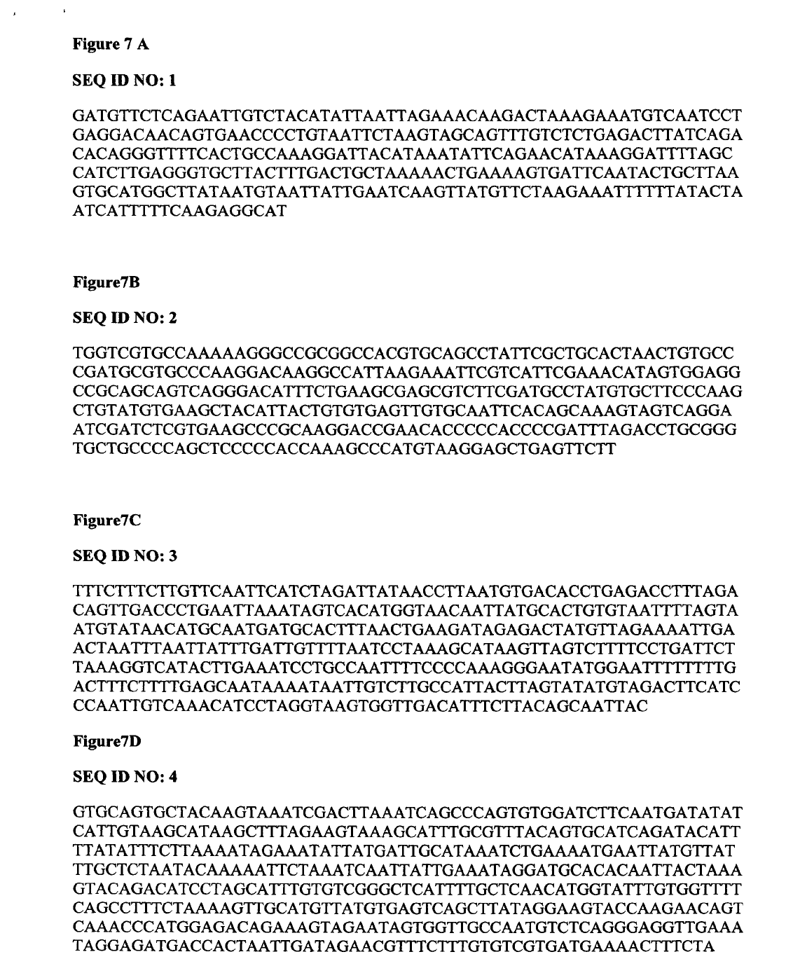

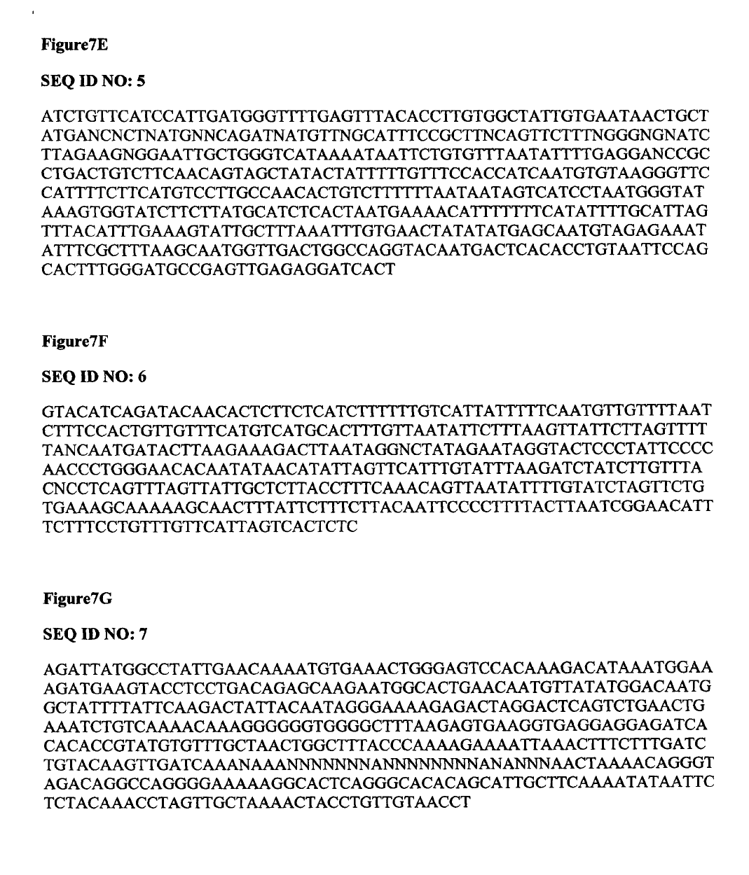

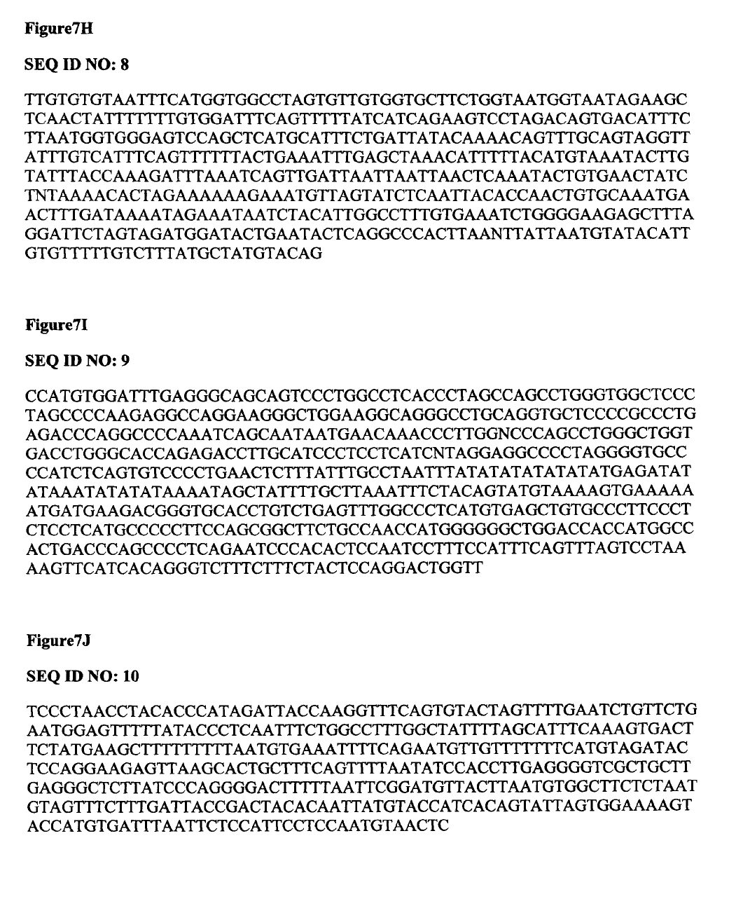

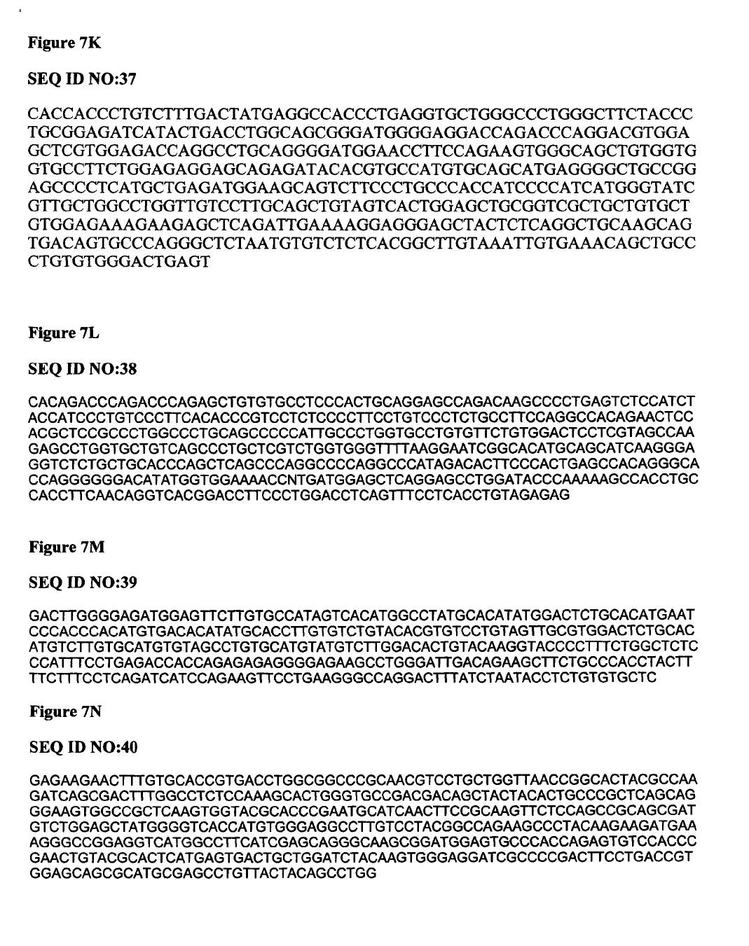

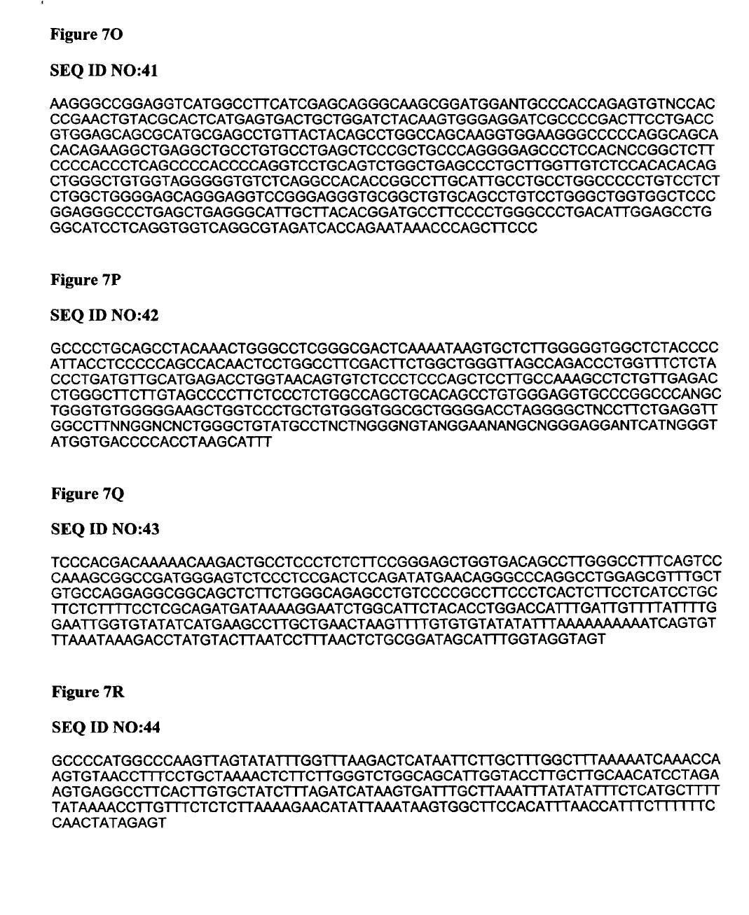

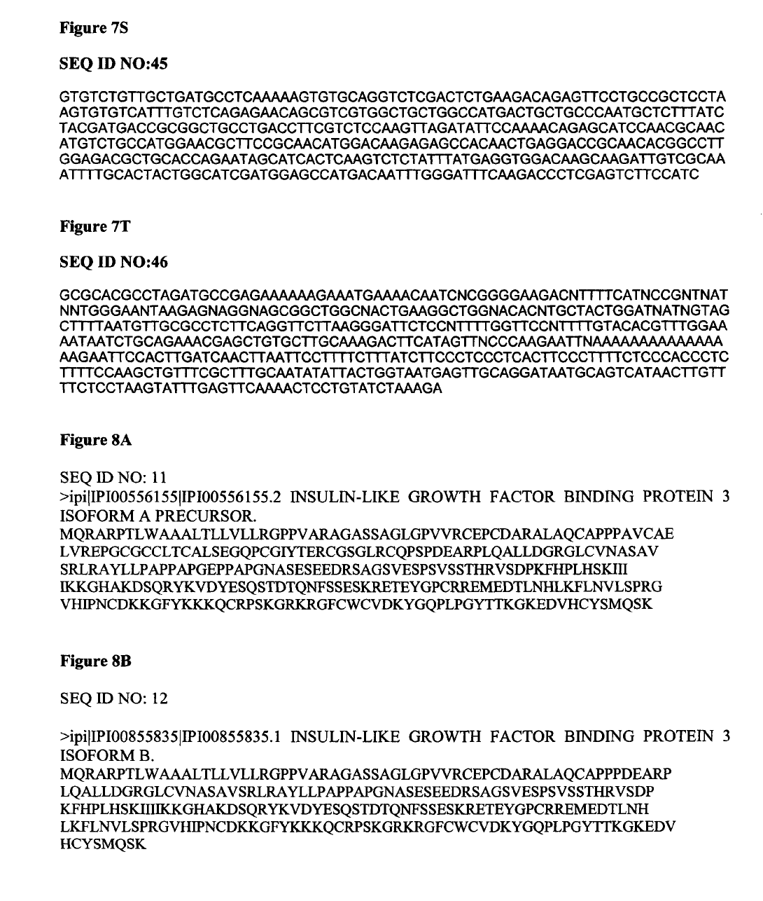

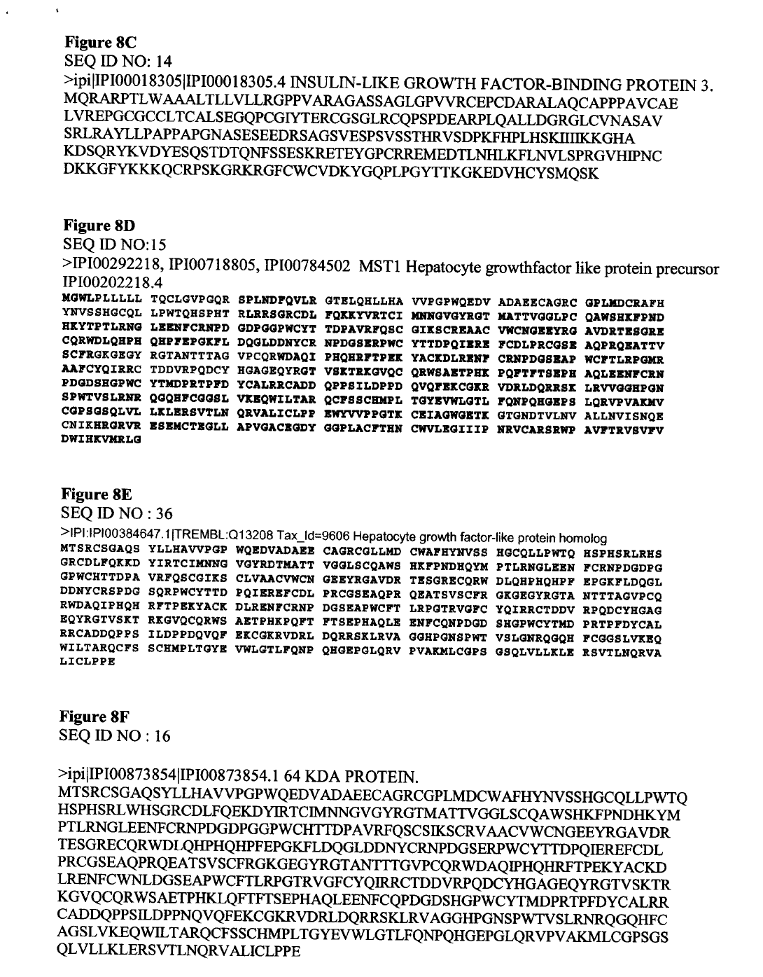

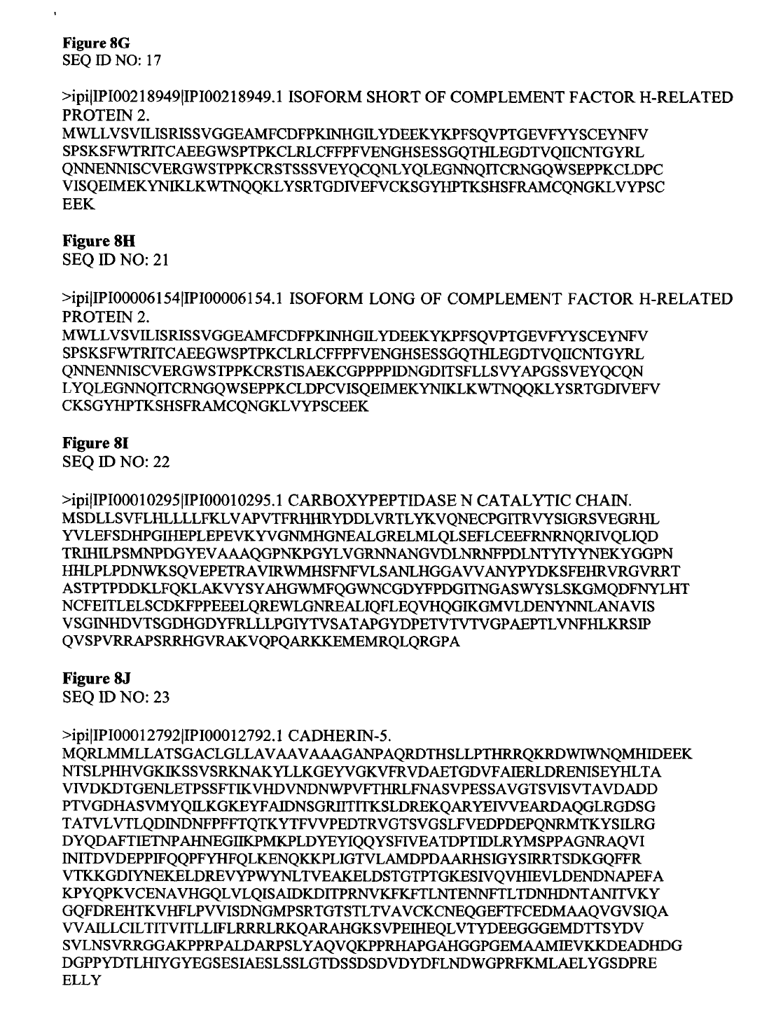

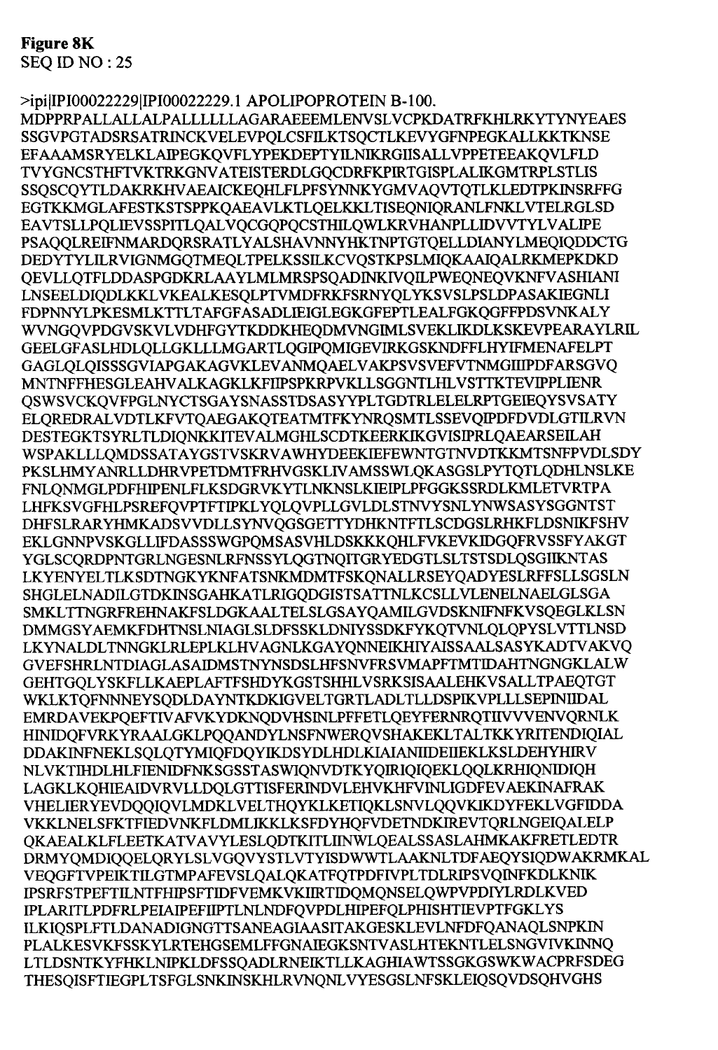

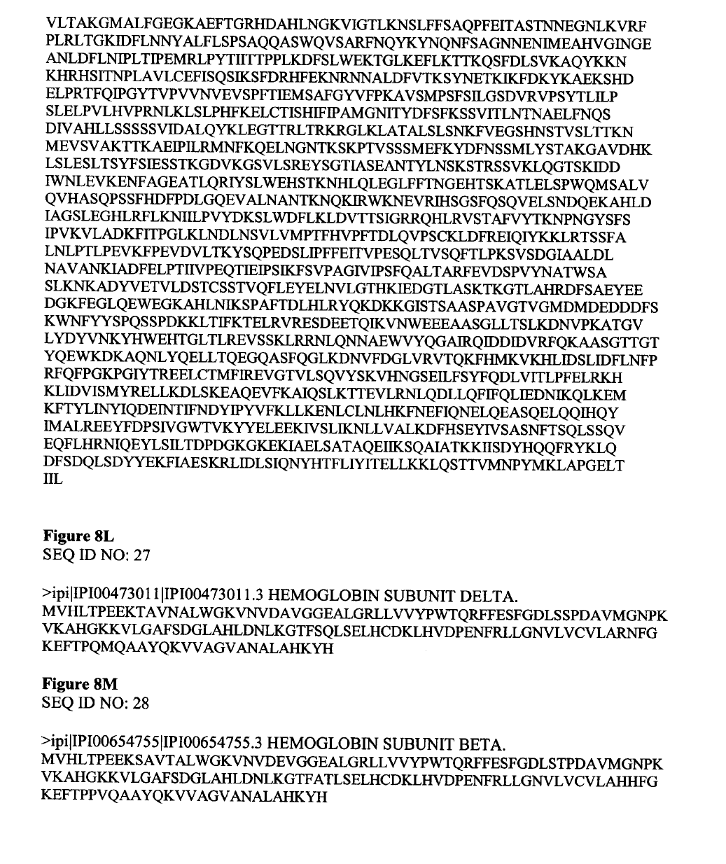

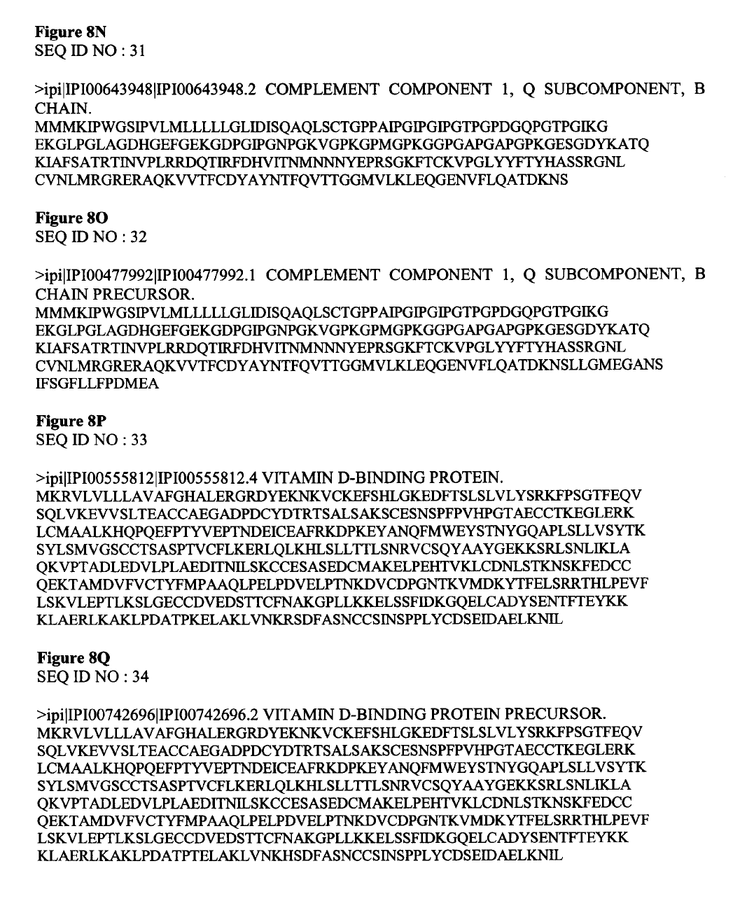

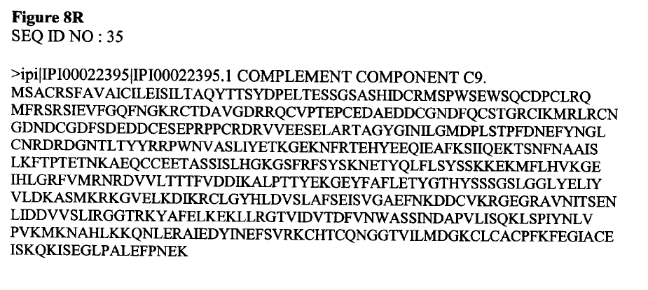

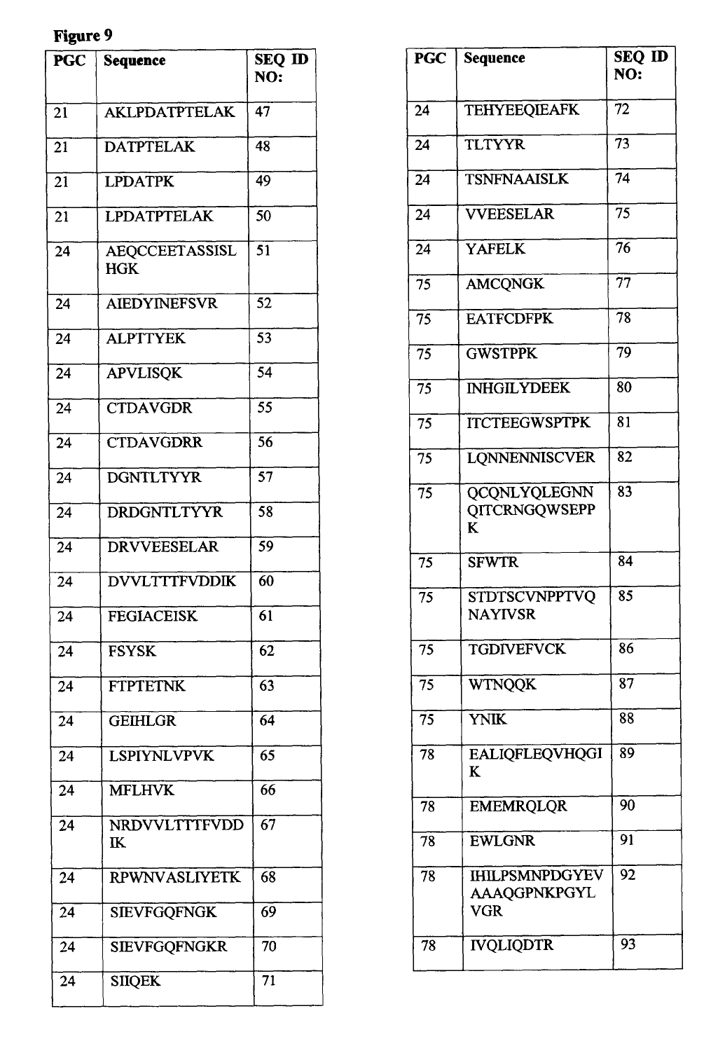

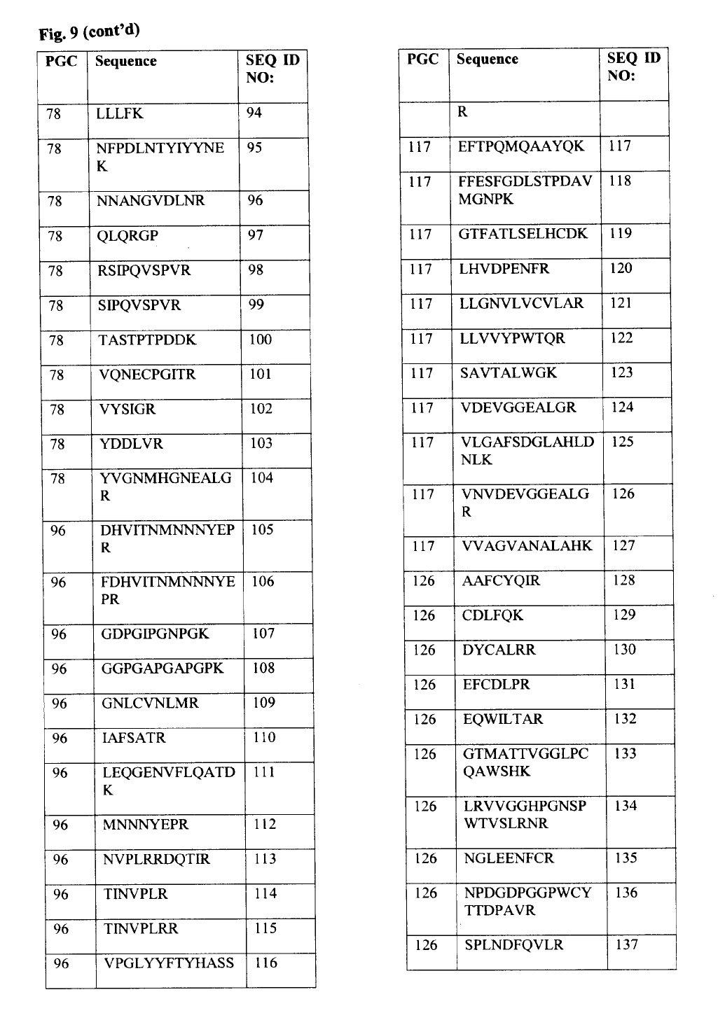

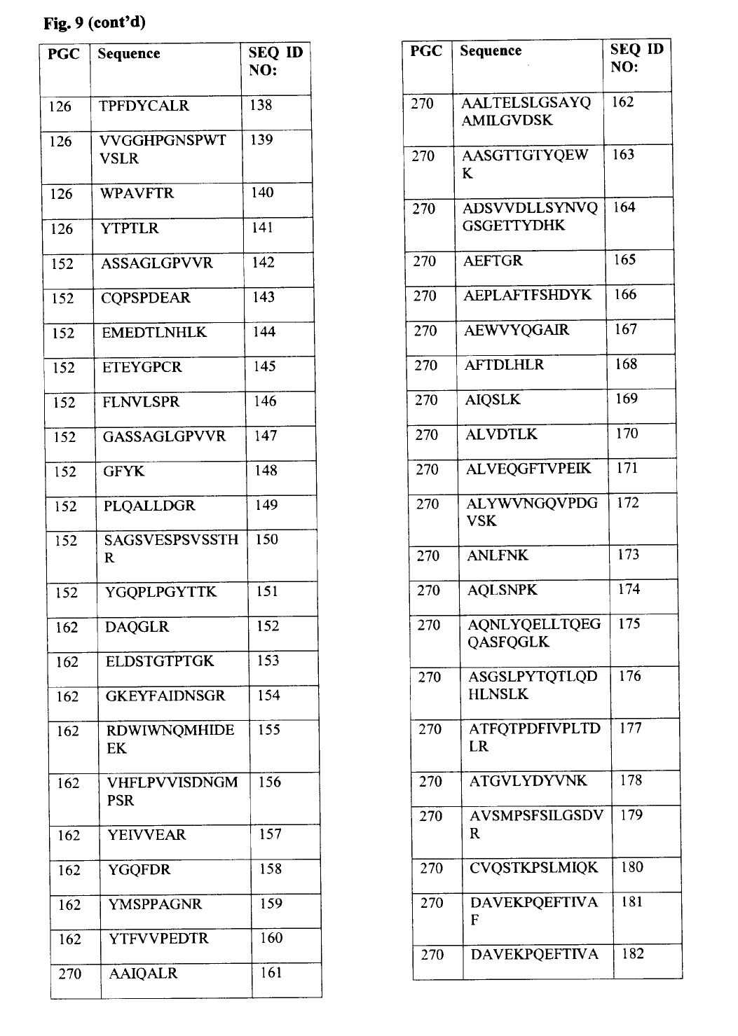

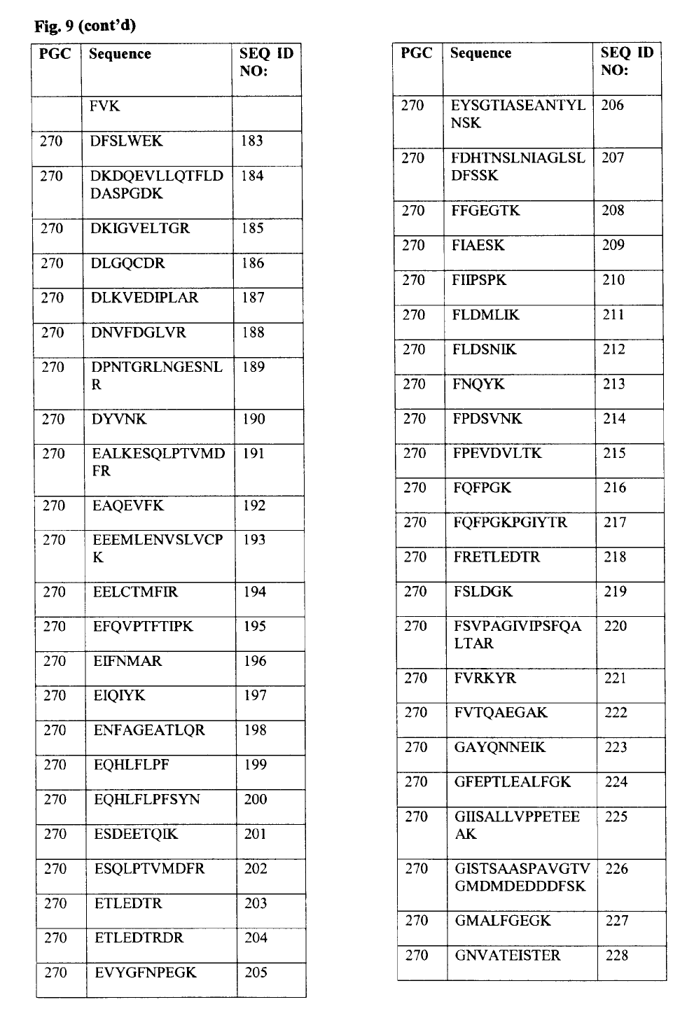

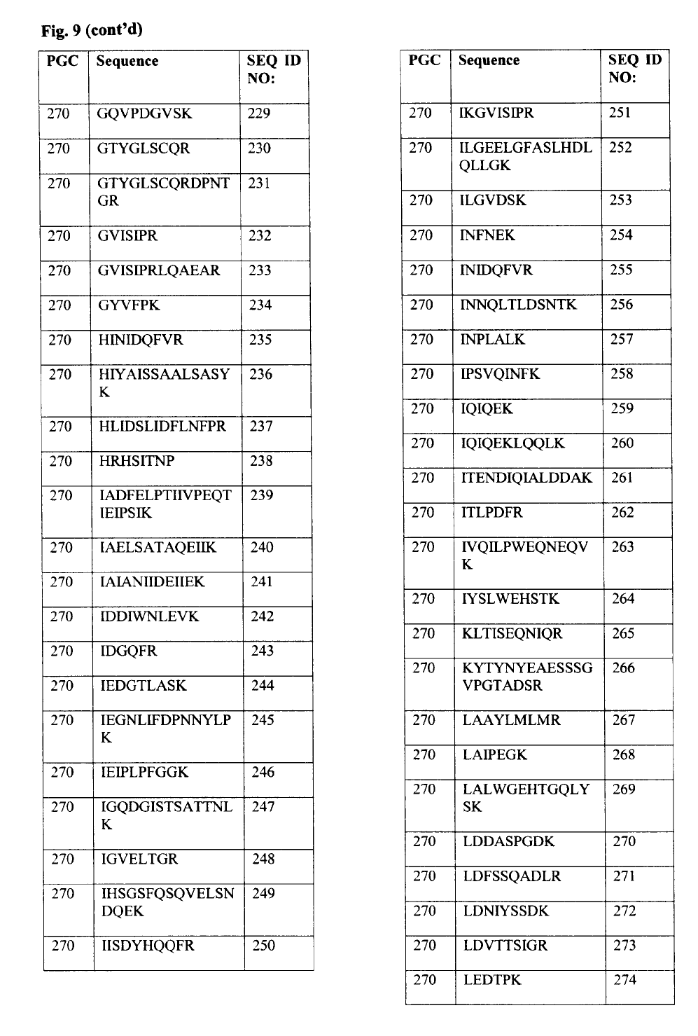

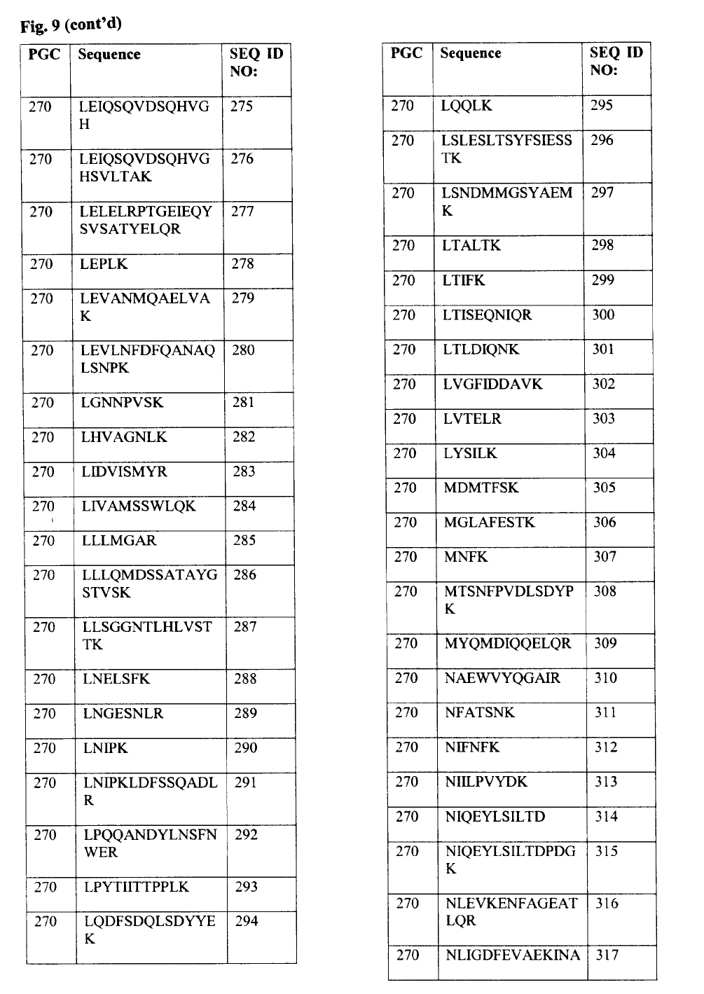

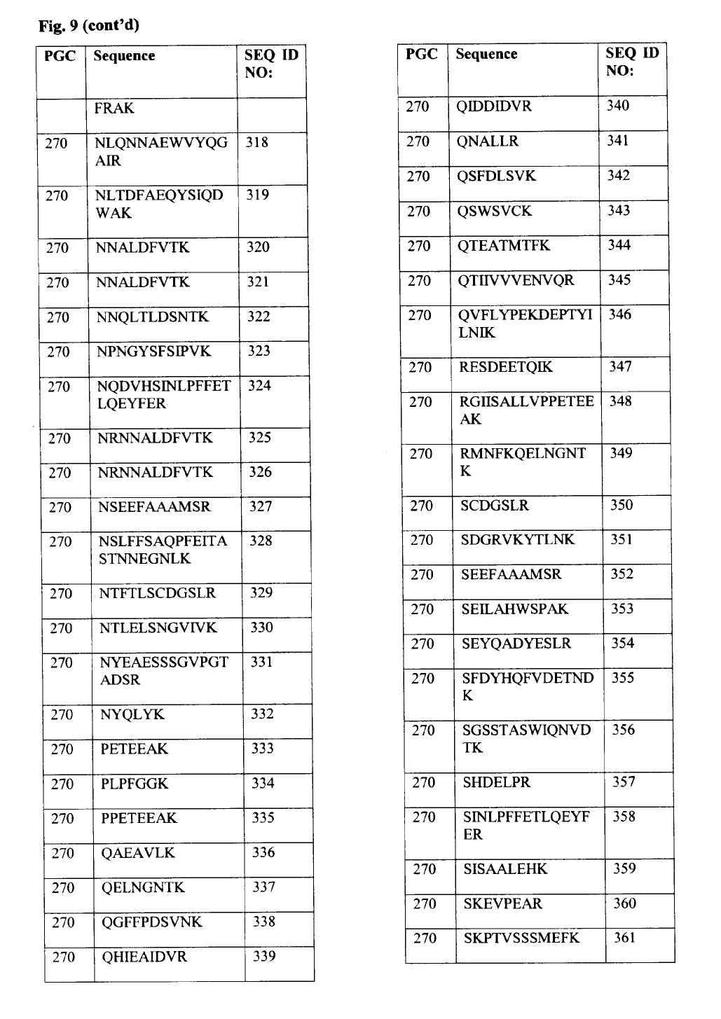

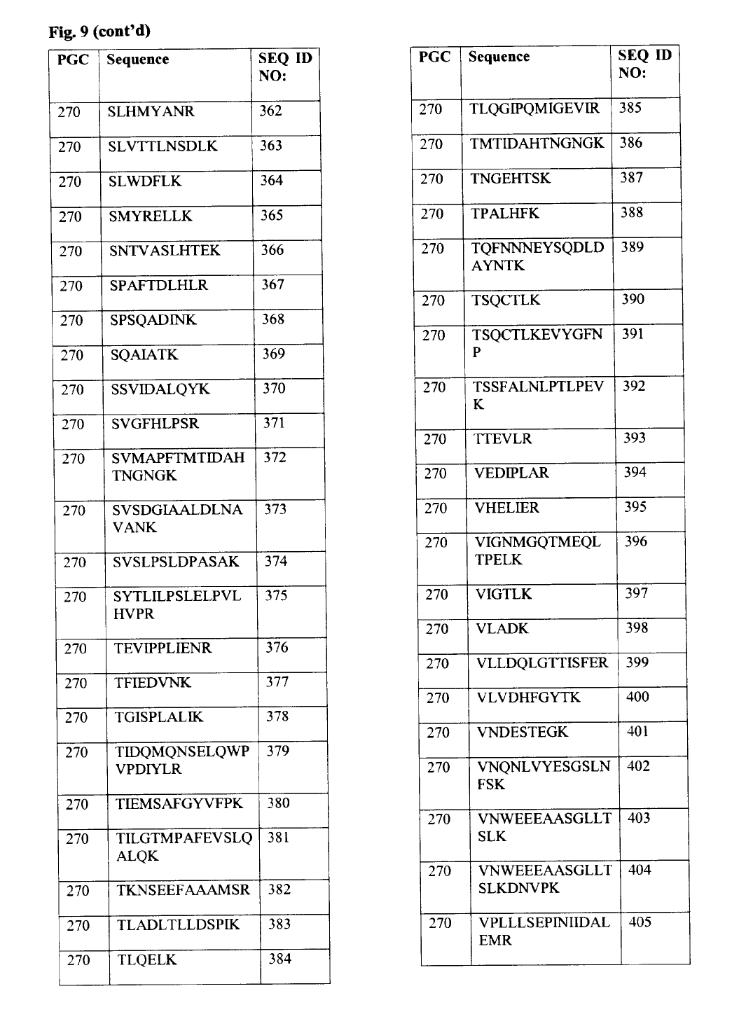

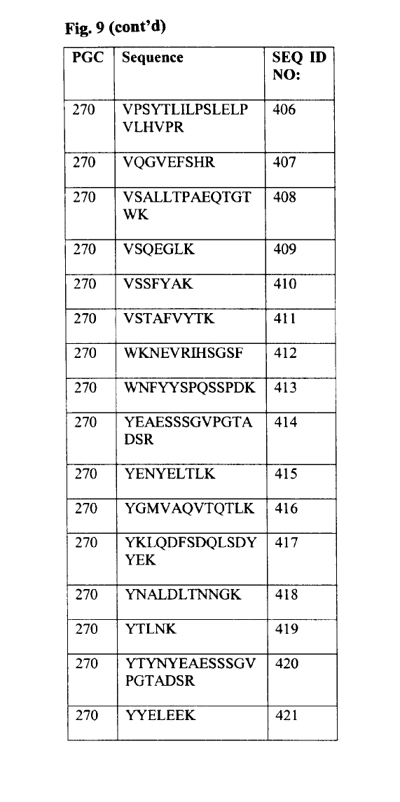

The combinatorial biomarker panel/classifier demonstrated an improvement in classification performance (FIG. 6). When the combinatorial classifier was applied to the same test cohort used in the genomic and proteomic internal validations, it was able to correctly discriminate between the CR and S samples with 100% sensitivity and 83% specificity (as compared to 83% sensitivity and specificity using the genomic and proteomic classifiers independently). The enhanced performance observed in our combinatorial panel is partly due to the fact that by applying both proteomic and genomic approaches, biomarkers found to be differentially expressed across the cohorts were less likely related to, or influenced by, platform specific bias.

Importantly, the genomic and proteomic panels, while each contain unique set of biomarkers, demonstrated comparable ability to discriminate between CR and S samples. The internal validation result for the combinatorial panel also highlights the potential advantage associated with a multi-platform approach.

The invention also provides for a kit for use in predicting or diagnosing a subject's rejection status. The kit may comprise reagents for specific and quantitative detection of comprising genomic marker OSBP2, CHPT1, RPS26, GBP3, KLRC1, ZCCHC2, 242907, CLEC2B, PDK4 and IFIT5, and proteomic markers encoded by CFHR2, CPN1, APOB, HBB, GC, C9, IGFBP3, MST1, CDH5 and C1QB, along with instructions for the use of such reagents and methods for analyzing the resulting data. The kit may be used alone for predicting or diagnosing a subject's rejection status, or it may be used in conjunction with other methods for determining clinical variables, or other assays that may be deemed appropriate. Instructions or other information useful to combine the kit results with those of other assays to provide a non-rejection cutoff index for the prediction or diagnosis of a subject's rejection status may also be provided.

### Other Embodiments

Alloreactive T Cell Profiling, Metabolomics

Alloreactive T-cell profiling and/or metabolite (“metabolomics”) profiling may be used in combination with genomic and/or proteomic profiling. Minor alterations in a subject's genome, such as a single base change or polymorphism, or expression of the genome (e.g. differential gene expression) may result in rapid response in the subject's small molecule metabolite profile. Small molecule metabolites may also be rapidly responsive to environmental alterations, with significant metabolite changes becoming evident within seconds to minutes of the environmental alteration—in contrast, protein or gene expression alterations may take hours or days to become evident. The list of clinical variables indicates several metabolites that may be used to monitor, for example, cardiovascular disease, obesity or metabolic syndrome—examples include cholesterol, homocysteine, glucose, uric acid, malondialdehyde and ketone bodies. Other non-limiting examples of small molecule metabolites are listed in Table 3.

TABLE 3
Metabolites identified and quantified in NMR spectra
of serum samples obtained from subject population.
Compound Name

| | |
|---|---|
| Glucose | Lactate |
| Glutamine | Alanine |
| Glycine | Proline |
| Glycerol | Valine |
| Taurine | Lysine |
| Citrate | Serine |
| Leucine | Ornithine |
| Creatinine | Tyrosine |
| Phenylalanine | Pyruvate |
| Histidine | Carnitine |
| Glutamate | Acetate |
| Isoleucine | Asparagine |
| Betaine | 3-Hydroxybutyrate |
| Creatine | Propylene glycol |
| 2-Hydroxybutyrate | Formate |
| Methionine | Choline |
| Acetone | |

Various techniques and methods may be used for obtaining a metabolite profile of a subject. The particulars of sample preparation may vary with the method used, and also on the metabolites of interest—for example, to obtain a metabolite profile of amino acids and small, generally water soluble molecules in the sample may involve filtration of the sample with a low molecular weight cutoff of 2-10 kDa, while obtaining a metabolite profile of lipids, fatty acids and other generally poorly-water soluble molecules may involve one or more steps of extraction with an organic solvent and/or drying and resolubilization of the residues. While some exemplary methods of detecting and/or quantifying markers have been indicated herein, others will be known to those skilled in the art and readily usable in the methods and uses described in this application.

Some examples of techniques and methods that may be used (either singly or in combination) to obtain a metabolite profile of a subject include, but are not limited to, nuclear magnetic resonance (NMR), gas chromatography (GC), gas chromatography in combination with mass spectroscopy (GC-MS), mass spectroscopy, Fourier transform MS (FT-MS), high performance liquid chromatography or the like. Exemplary methods for sample preparation and techniques for obtaining a metabolite profile may be found at, for example, the Human Metabolome Project website (Wishart D S et al., 2007. Nucleic Acids Research 35:D521-6).

The present invention will be further illustrated in the following examples. However it is to be understood that these examples are for illustrative purposes only, and should not be used to limit the scope of the present invention in any manner.

Methods

### Subjects and Specimens

Subjects were enrolled according to Biomarkers in Transplantation standard operating procedures. Subjects waiting for a cardiac transplant at the St. Paul's Hospital, Vancouver, BC were approached by the research coordinators and 39 subjects who consented were enrolled in the study. All cardiac transplants are overseen by the British Columbia Transplant (BCT) and all subjects receive standard immunosuppressive therapy. Blood samples from consented subjects were taken before transplant (baseline) and at weeks 1, 2, 3, 4, 8, 12, 26 and every 6 months up to 3 years post-transplant. Additionally, blood samples were taken from consented subjects at single time-points between 1 and 5 years post-transplant. Blood was collected in PAXGene™ tubes, placed in an ice bath for delivery, frozen at −20° C. for one day and then stored at −80° C. until RNA extraction.

Heart transplant subject data was reviewed and 25 subjects were selected for analysis. A total of 40 blood samples from single or time series samples between years 1 and 13 post-transplant were selected for RNA extraction and microarray analysis. Four baseline blood samples were also processed.

Two types of subjects were enrolled: those who were waiting for a transplant (De Novo), and those who were coming in for their yearly exam (Existing) between March 2005 and February 2008. For the De Novo subjects, serial blood and urine samples were collected from pre-transplant (baseline), at weeks 1, 2, 3, 4, 8, 12 and 26, every 6 months for up to 3 years post-transplant, and at the time of suspected rejection. For the existing subjects, a single sample was collected at least one year post-transplant, during routine post-transplant check-ups. Blood samples from healthy individuals served as controls for the genomic (whole blood) and proteomic (plasma) analyses.

Both De Novo and Existing transplant subjects received basilimax induction followed by standard triple immunosuppressive therapy consisting of cyclosporine, predinosone and mycophenolate mofetil. Cyclosporin was replaced by tacrolimus for women, and by sirolomus in the case of renal impairment. Subjects who had a 2R or 3R ISHLT rejection grade episode within 3 months post-transplant received methylprednisolone.

Screening for chronic rejection (CR) was routinely performed using dobutamine stress echocardiography, coronary angiography and intravascular ultrasounds (IVUS) according to the ‘Protocol for Long-term Surveillance of Cardiac Allograft Vasculopathy’ guidelines, as established by St. Paul's Heart Centre. Diagnoses of chronic rejections were made based on chart reviews (i.e., angiography and/or IVUS measurements) at time points corresponding to the blood sample collection date. For the purpose of this investigation, CR and stable (S) were identified based on clinical confirmation and defined as ≧50% and ≦25% stenosis, respectively.

Analysis Population

The objective of this study was to identify whole blood genomic and plasma proteomic biomarkers that differentiate between chronic rejection (CR) [clinical confirmation and more than 50% stenosis] and stable (S) [clinical confirmation and less than 25% stenosis] samples. A total of 25 blood samples, collected between year one and year five post-transplantation from 17 patients (11 De Novo, 6 Existing), were selected for genomic and proteomic analyses. Subject samples were divided into training and test cohorts. The training cohort consisted of 13 samples collected at one year (7 CR and 6 S) and two years (1 CR) post-transplant from 13 patients. The test cohort consisted of 12 samples (6 CR and 6 S). Seven of these samples (2 CR and 5 S) were collected from the 5 training cohort subjects at later time points, and 5 (4 CR and 1 S) were collected from 4 non-training cohort subjects. Patient demographics were comparable between the training and test cohorts (Table 4).

TABLE 4
Demographics of cardiac transplant subject cohorts

| | Training Subjects | Test Subjects |
|---|---|---|
| | (n = 13) | (n = 9) |
| Age (mean, standard deviation in years) | 52 ± 13 | 54 ± 11 |
| Gender (n male) | 10 | 7 |
| Ethnicity (n) | | |
| Caucasian | 13 | 9 |
| Primary Disease (n) | | |
| Ischemic Heart Disease | 4 | 3 |
| Non-ischemic Cardiomyopathy | 7 | 4 |
| Other | 2 | 2 |

RNA Extraction and Microarray Analysis

RNA extraction was performed on thawed samples using the PAXgene™ Blood RNA Kit [Cat #762134] to isolate total RNA. Between 4 and 10 μg of RNA was routinely isolated from 2.5 ml whole blood and the RNA quality confirmed using the Agilent BioAnalyzer. Samples with 1.5 μg of RNA, an RIN number >5, and A240/A280>1.9 were packaged on dry ice and shipped by Federal Express to the Microarray Core (MAC) Laboratory, Children's Hospital, Los Angeles, Calif. for Affymetrix microarray analysis. The microarray analysis was performed by a single technician at the CAP/CLIA accredited MAC laboratory. Nascent RNA was used for double stranded cDNA synthesis. The cDNA was then labeled with biotin, fragmented, mixed with hybridization cocktail and hybridized onto GeneChip Human Genome U133 Plus 2.0 Arrays. The arrays were scanned with the Affymetrix System in batches of 48 with an internal RNA control made from pooled normal whole blood. Microarrays were checked for quality issues using Affymetrix version 1.16.0 and affyPLM version 1.14.0 BioConductor packages (Bolstad, B., Low Level Analysis of High-density Oligonucleotide Array Data: Background, Normalization and Summarization. 2004, University of California, Berkeley; Irizarry et al. 2003. Biostatistics 4(2): 249-64). The arrays with lower quality were repeated with a different RNA aliquot from the same time point.

Proteomics:

Plasma Processing, Depletion, Trypsin Digest and ITRAQ Labelling

Blood samples were collected in EDTA tubes, immediately stored on ice and processed within 2 hours for plasma before storage. Plasma was obtained from each whole blood sample through centrifugation, aliquoted and stored at −70° C. until the proteomic analysis. One hundred micrograms of total protein from each sample was prepared with iTRAQ reagents. Briefly, plasma samples were depleted of the 14 most abundant plasma proteins (albumin, fibrinogen, transferin, IgG, IgA, IgM, haptoglobin, α2-macroglobulin, α1-acid glycoprotein, α1-antitrypsin, Apoliprotein-I, Apoliprotein-II, complement C3 and Apoliprotein B) by immuno-affinity chromatography (Genway Biotech; San Diego, Calif.), trypsin digested with sequencing grade modified trypsin (Promega; Madison, Wis.) and labelled with iTRAQ reagents according to manufacturer's (Applied Biosystems; Foster City, Calif.) protocol. Labelled samples were pooled and acidified to pH 2.5-3.0. iTRAQ labeled peptides were separated by strong cation exchange chromatography (PolyLC Inc., Columbia, Md. USA). The resulting labelled peptides were pooled, further separated by reverse phase chromatography (Michrom Bioresources Inc., Auburn, Calif. USA) and spotted directly onto 384 spot MALDI ABI 4800 plates, 4 plates per experiment, using a Probot microfration collector (LC Packings, Amsterdam, Netherlands).

Mass Spectrometry and Data Processing

Peptides spotted onto MALDI plates were analyzed by a 4800 MALDI TOF/TOF analyzer (Applied Biosystems; Foster City, Calif.) controlled using 4000 series Explorer version 3.5 software. The mass spectrometer was set in the positive ion mode with an MS/MS collision energy of 1 keV. A maximum of 1400 shots/spectrum were collected for each MS/MS run, causing the total mass time to range from 35 to 40 hours. Peptide identification and quantitation was carried out by ProteinPilot™ Software v2.0 (Applied Biosystems/MDS Sciex, Foster City, Calif. USA) with the integrated new Paragon™ Search Algorithm (Applied Biosystems) and Pro Group™ Algorithm. Database searching was performed against the International Protein Index (IPI HUMAN v3.39) (Kersey et al., 2004. Proteomics 4:1985-1988). The precursor tolerance was set to 150 ppm and the iTRAQ fragment tolerance was set to 0.2 Da. Identification parameters were set for trypsin cleavages, cysteine alkylation by MMTS, with special factors set at urea denaturation and an ID focus on biological modifications. The detected protein threshold was set at 85% confidence interval.

Proteomic Analysis

Pro Group™ Algorithm (Applied Biosystems) assembled the peptide evidence from the Paragon™ Algorithm into a comprehensive summary of the proteins in the sample.

The set of identified proteins from each iTRAQ run were organized into protein groups to avoid redundancies. Each iTRAQ run involved three subject samples plus one pooled control sample—the control was consistently labelled with iTRAQ reagent 114, while the subject samples were randomly labelled between reagents 115, 116 and 117. Relative protein levels (levels of labels 115, 116 and 117 relative to 114, respectively) were estimated for each protein group by Protein Pilot using the corresponding peptide ratios. As each protein group may consist of more than one identified protein, an in-house algorithm, called Protein Group Code Algorithm (PGCA) was employed to link protein groups across all iTRAQ experiments. PGCA assigns an identification code to all the protein groups within each iTRAQ run and a common code to similar protein groups across runs. The latter code, also referred to as the protein group code (PGC), was then used to match proteins across different iTRAQ runs. This process ensures common identifier nomenclature for related proteins and protein families across all experimental runs for comparison purposes.

Statistical Analysis

Single time-point samples from subjects with either chronic rejection (n=6) or a stable course (n=7) at one year post-transplant were diagnosed using IVUS, angiography, dobutamine stress echocardiography and/or clinical review. This “clean cohort” was used for discovery of the chronic rejection diagnostic biomarker panel.

Statistical analysis for both genomic and proteomic data was performed using a “funnel” approach, which was implemented using R version 2.6.0.

Step 1: Pre-filtering
All probe sets on the microarray were filtered to provide a pre-filtered probe set;
All protein groups in the PGCA dictionary were filtered to provide pre-filtered protein groups.

Step 2: Robust t-test
All pre-filtered pro sets and protein groups were subjected to a Robust t-test to provide the differentially expressed (DE) probe set or DE protein groups, respectively.

Step 3: Panel selection
The DE probe sets or protein groups were further analyzed to provide for the genomic biomarker panel or the proeomic biomarker panel, respectively

Step 4: Dimension reduction
The genomic and proteomic biomarker panels were poled to provide a combinatorial biomarker panel.

The statistical analysis was performed using SAS version 9.1, R version 2.6.1 and BioConductor version 2.1 (Gentleman, R., et al., Genome Biology, 2004. 5: p. R80). Robust Multi-array Average (RMA) (Bolstad, et al. Bioinformatics, 2003. 19(2): p. 185-93) technique was used for background correction, normalization and summarization as available in the Affymetrix BioConductor package. A noise minimization was then performed; probe sets with expression values consistently lower than 50 across at least 3 samples were considered as noise and eliminated from further analysis. The remaining probe sets were analyzed using three different moderated T-tests. Two of the methods are available in the Linear Models for Microarray data (limma) BioConductor package—robust fit combined with eBayes and least square fit combined with eBayes. The third statistical analysis method, Statistical Analysis of Microarrays (SAM), is available in the same BioConductor package. A gene was considered statistically significant if it had a false discovery rate (FDR) <0.05 in all three methods and a fold change >2 in all three moderated T-tests (fold change >1.6 for alloreactive T-cells) (Smyth, G., Limma: linear models for microarray data, in Bioinformatics and Computational Biology Solutions using R and Bioconductor, R. Gentleman, et al., Editors. 2005, Springer: New York). The biomarker panel genes were identified by applying Stepwise Discriminant Analysis (SDA) with forward selection on the statistically significant probe sets. Linear Discriminant Analysis (LDA) was used to train and test the biomarker panel as a classifier.

Genomics

In step 1, the Robust Multi-array Average (RMA) technique was used for background correction, normalization and summarization (Affy BioConductor package version 1.6.7). To reduce noise, probe sets with consistently low expression values across all samples were eliminated from further analysis. The remaining probe sets were analyzed using a robust moderated t-test (Step 2) with limma BioConductor package, version 1.9.6. Probe sets with a False Discovery Rate (FDR) <10% were considered statistically significant. Biomarker panel genes were identified by applying a more stringent cut-off criterion, FDR <5% and a fold change >2 (Step 3). An internal validation was performed using Linear Discriminant Analysis (LDA) to estimate the ability of the genomic panel to discriminate CR from S samples.

Proteomics

In step 1, PGCs that were not detected in at least ⅔ of the patients within each group (i.e., 5 out of 7 ARs and 4 out of 6 NRs) were eliminated from further analysis. The remaining protein groups were analyzed using a robust moderated t-test (step 2) with the limma Bioconductor package, version 1.9.6. Protein group codes with differential relative concentrations (relative to pooled control's levels) between the CR and S samples were identified and considered for the proteomic biomarker panel. In step 3, a more rigorous cut-off was then applied (p-value <0.03) to select the biomarker panel proteins. Internal validation was performed using Linear Discriminant Analysis (LDA) to estimate the ability of the proteomic panel to discriminate CR from S samples. In LDA, the relative concentration for each protein undetected in patient sample(s) and/or pooled control was imputated using the average relative concentration calculated from other samples in the training cohort.

Functional Enrichment

Functional enrichment of the differentially expressed genes and proteins identified (Step 2) were examined using FatiGO (Al-Shahrour et al., 2007. Nucleic Acid Research 35:W91-96), available in version 3 of Babelomics (Al-Shahrour et al., 2006. Nucleic Acids Research W472-476), a suite of web-based tools designed for functional analysis.

Combinatorial Analysis

To create a combinatorial biomarker panel, a subset of proteins and probe sets were separately identified using stepwise discriminant analysis (SDA) that maximized the classification accuracy in a leave-one-out cross validation (Weihs, C., Ligges, U., Luebke, K. and Raabe, N. (2005). klaR Analyzing German Business Cycles. In Baier, D., Decker, R. and Schmidt-Thieme, L. (eds.). Data Analysis and Decision Support, 335-343, Springer-Verlag, Berlin). (Step 3). The resulting subsets of proteins and probe sets were then combined into a combinatorial biomarker panel. An internal validation was performed using Linear Discriminant Analysis (LDA) to estimate the ability of the combinatorial panel to discriminate CR from S samples. In both SDA and LDA, for each protein undetected in some patient samples and/or pooled control, an average relative concentration from other samples in the training cohort was used as a replacement.

### Example 1

Following normalization and pre-filtering, 25,215 probe sets remained and were included in the subsequent analysis (Step 2) using the training cohort samples. Using robust-test, a total of 106 probe sets were identified as having FDR <10%. A heatmap was constructed for these probe sets to visualize the relative expression levels between CR and S samples (FIG. 4). In addition, over representation analysis was carried out to observe the type of biological and molecular processes encompassed by the differentially expressed genes compare to the rest of the genes present on the microarray. The significantly enriched gene ontology (GO) terms were identified, and those with p-value <0.05 have been summarized in Table 5.

TABLE 5
Statistically significant gene ontology terms as identified by
enrichment analysis (FatiGo) for genomic expression profiling.

| Process or response | GeneOntology term (GO term) |
|---|---|
| Immune response | GO: 0006955 |
| response to biotic stimulus | GO: 0009607 |
| humoral immune response | GO: 0006959 |
| response to other organism | GO: 0051707 |
| lipid metabolic process | GO: 0006629 |
| antimicrobial humoral response | GO: 0019730 |
| cellular lipid metabolic process | GO: 0044255 |
| lipid transport | GO: 0006869 |
| carbohydrate binding | GO: 0030246 |
| structural constituent of ribosome | GO: 0003735 |
| sugar binding | GO: 0005529 |
| diacylglycerol binding | GO: 0019992 |

P-value < 0.05. The GO terms (biological process and molecular functions) shown are between GO levels 3 and 5.

From the 106 biomarker candidates, 22 differentially expressed probe sets were identified, each of which demonstrated at a least 2-fold difference between samples from chronic rejection patients (CR) and those from the clean cohort (non-rejection patients (NR)) (Table 6). A subset of 10 probe sets were identified using a more stringent criteria (FDR <5% and fold change >2). These 10 probe sets make up the genomic biomarker panel—CHPT1, LOC644166/LOC644191/LOC728937/RPS26, GBP3, KLRC1/KLRC2, ZCCHC2, 242907_at, CLEC2B, PDK4, OSBP2, IFIT5 and allow for categorization of each sample as CR or NR. One of the biomarker panel probe sets/genes (OSBP2) was downregulated in CR relative to S, while the rest (CHPT1, RPS26, GBP3, KLRC1, ZCCHC2, 242907_at, CLEC2B, PDK4, IFIT5) were upregulated. 242907_at (unknown 4) is an unnamed target that exhibited at least a two-fold increase.

An internal validation was performed to estimate the ability of the genomic biomarker panel to jointly classify 12 new samples (6 CR and 6 S). One CR and one S sample were misclassified, which corresponds to 83% sensitivity and specificity for the genomic chronic cardiac allograft rejection biomarker panel.

TABLE 6
Chronic, whole-blood cardiac allograft rejection genomic biomarkers.

| | | | | | | Regulation | Representative |
|---|---|---|---|---|---|---|---|
| | | | Ref Seq Transcript | log2 | Fold | in CR | sequence (SEQ |
| Affy ID | GeneSymbol | GeneTitle | ID | (CR/NR) | Change | versus S | ID NO:) |
| 230364_at | CHPT1 | choline | NM_020244 | −2.00 | 3.99 | down | 1 |
| | | phosphotransferase 1 | | | | | |
| 217753_s_at | LOC644166/ | ribosomal protein | NM_001029; | −1.95 | 3.86 | down | 2 |
| | LOC644191/ | S26 /// similar to | NM_001093731 | | | | |
| | LOC728937/ | 40S ribosomal | XM_001130384; | | | | |
| | RPS26 | protein S26 | XM_001132755; | | | | |
| | | | XM_930072; | | | | |
| | | | XM_941927 | | | | |
| 223434_at | GBP3 | guanylate binding | NM_018284 | −1.78 | 3.44 | down | 3 |
| | | protein 3 | | | | | |
| 206785_s_at | KLRC1/ | “killer cell lectin- | NM_002259; | −1.37 | 2.59 | down | 4 |
| | KLRC2 | like receptor | NM_002260; | | | | |
| | | subfamily C, | NM_007328; | | | | |
| | | member 1 /// killer | NM_213657; | | | | |
| | | cell lectin-like | NM_213658 | | | | |
| | | receptor subfamily | | | | | |
| | | C, member 2” | | | | | |
| 233425_at | ZCCHC2 | “zinc finger, | NM_017742 | −1.20 | 2.30 | down | 5 |
| | | CCHC domain | | | | | |
| | | containing 2” | | | | | |
| 242907_at | “Unknown” | — | *Unknown | −1.15 | 2.23 | down | 6 |
| 1556209_at | CLEC2B | “C-type lectin | NM_005127 | −1.26 | 2.39 | down | 7 |
| | | domain family 2, | | | | | |
| | | member B” | | | | | |
| 225207_at | PDK4 | “pyruvate | NM_002612 | −1.66 | 3.17 | down | 8 |
| | | dehydrogenase | | | | | |
| | | kinase, isozyme 4” | | | | | |
| 223432_at | OSBP2 | oxysterol binding | NM_030758 | 1.05 | 2.06 | up | 9 |
| | | protein 2 | | | | | |
| 203595_s_at | IFIT5 | interferon-induced | NM_012420 | −1.04 | 2.06 | down | 10 |
| | | protein with | | | | | |
| | | tetratricopeptide | | | | | |
| | | repeats 5 | | | | | |
| 211529_x_at | HLA-G | “HLA-G | NM_002127 | −0.42 | 1.34 | down | 37 |
| | | histocompatibility | | | | | |
| | | antigen, class I, G” | | | | | |
| 217045_x_at | NCR2 | natural | NM_004828 | 0.36 | 1.28 | up | 38 |
| | | cytotoxicity | | | | | |
| | | triggering receptor 2 | | | | | |
| 204891_s_at | LCK | lymphocyte- | NM_001042771; | −0.56 | 1.48 | down | 39 |
| | | specific protein | NM_005356 | | | | |
| | | tyrosine kinase | | | | | |
| 1555613_a_at | ZAP70 | zeta-chain (TCR) | NM_001079; | −0.62 | 1.54 | down | 40 |
| | | associated protein | NM_207519 | | | | |
| | | kinase 70 kDa | | | | | |
| 214032_at | ZAP70 | zeta-chain (TCR) | NM_001079; | −0.89 | 1.85 | down | 41 |
| | | associated protein | NM_207519 | | | | |
| | | kinase 70 kDa | | | | | |
| 205536_at | VAV2 | vav 2 guanine | NM_003371 | 0.45 | 1.37 | up | 42 |
| | | nucleotide | | | | | |
| | | exchange factor | | | | | |
| 223049_at | GRB2 | growth factor | NM_002086; | −0.41 | 1.33 | down | 43 |
| | | receptor-bound | NM_203506 | | | | |
| | | protein 2 | | | | | |
| 230337_at | SOS1 | son of sevenless | NM_005633 | −0.71 | 1.64 | down | 44 |
| | | homolog 1 | | | | | |
| | | (Drosophila) | | | | | |
| 200950_at | ARPC1A | “actin related | NM_006409 | −0.31 | 1.24 | down | 45 |
| | | protein ⅔ | | | | | |
| | | complex, subunit | | | | | |
| | | 1A, 41 kDa” | | | | | |
| 213513_x_at | ARPC2 | “actin related | NM_005731; | −0.50 | 1.42 | down | 46 |
| | | protein ⅔ | NM_152862 | | | | |
| | | complex, subunit | | | | | |
| | | 2, 34 kDa” | | | | | |

*242907_at - A nucleotide BLAST search with SEQ ID NO: 6 demonstrates 98% identity with nucleotides 58544174-59543814 of NT_032977.8 (Human chromosome 1 genomic contig, reference assembly). Features flanking this part of subject sequence include: 1603 bp at 5′ side: guanylate binding protein 2, interferon-inducible and 42939 bp at 3′ side: guanylate binding protein 1, interferon-inducible, 67 kD.

### Example 2

### Biological Pathways

Using a combination of bioinformatics and literature-based approaches, various pathways have been identified based on selected differentially expressed genes. Without wishing to be bound by theory, interactions between them have also been elucidated in our current results. FIG. 3 illustrates a pathway-based relationship between the biomarkers NKG2A (KLRC1), NKG2C (KLRC2), PDK4 and CHPT1.

Without wishing to be bound by theory, interactions between the biomarker genes and/or gene products may include:

### 1. NKG2C (KLRC2)→CD94→NKG2A (KLRC1)

NKG2C (KLRC2)→CD94 (Ding et al 1999. Scand. J Immunol 49:459-65; Gunturi et al 2004. Immunol. Res 30:29-34)
CD94→NKG2A (KLRC1) (Brooks et al 1997. J Exp Med 185:795-800; Brooks et al 1999. J. Immunol. 162:305-13; Dulphy et al 2002. Int Immunol 14:471-9)

### 2. NKG2C/NKG2A (KLRC2/KLRC1)→SHP1→ESR1→PDK4 and CHPT1

NKG2C/NKG2A (KLRC2/KLRC1)→SHP1 (Lin Chua et al 2002. Cell Immunol. 219:57-70; Le Drean et al 1998. Eur J Immunol 28:264-76)
SHP1→ESR1 (Grimaldi et al 2002. 109:1625-33)

### 3. ESR1→PDK4 and CHPT1

(Araki et al 2006. FEBS J. 273:1669-80; Laganiere et al 2005. Proc Natl Acad Sci USA. 102:11651-6)

### Example 3

### Proteomic Analysis Results

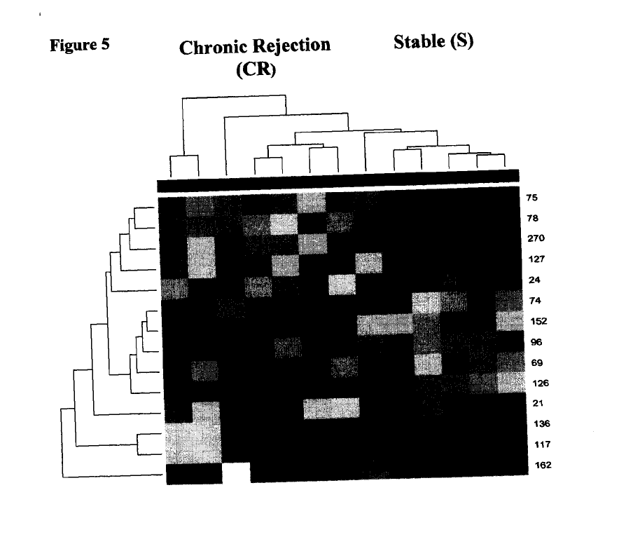

A total of ˜2500 protein groups codes (PGC) were found in at least one of the 13 samples included in the training cohort. These PGCs were pre-filtered (Step 1)—PGCs which were detected in at least ⅔ of the 7 CR and 6 S samples (i.e., 5 CR and 6 S samples) were used for further analysis. Statistical analysis identified 14 of the 129 analyzed proteins with differential relative concentrations with p-value <0.05 (Step 3). A heatmap was constructed to visualize the performance of these significant PGCs in discriminating CR from S samples (FIG. 5). Over representation analysis was also carried out to explore the biological and molecular functions of all the proteins belonging to these protein group codes. The significantly enriched GO terms with p-value <0.05 are shown in Table 7.

TABLE 7
Statistically significant gene ontology terms as identified by enrichment
analysis (FatiGo) for proteomic expression profiling.

| Process or response | GeneOntology term (GO term) |
|---|---|
| response to external stimulus | GO: 0009605 |
| defense response | GO: 0006952 |
| immune response | GO: 0006955 |
| immune effector process | GO: 0002522 |
| humoral immune response | GO: 0006959 |
| innate immune response | GO: 0045087 |
| response to wounding | GO: 0009611 |
| transport | GO: 0006810 |
| adaptive immune response | GO: 0002250 |
| nitric oxide metabolic process | GO: 0046209 |
| regulation of immune system process | GO: 0002682 |
| inflammatory response | GO: 0006954 |
| vitamin transport | GO: 0051180 |
| leukocyte mediated immunity | GO: 0002443 |
| interaction with host | GO: 0051701 |
| oxygen transporter activity | GO: 0005344 |
| oxygen binding | GO: 0019825 |
| lipid transporter activity | GO: 0005319 |
| vitamin transporter activity | GO: 0051183 |
| steroid binding | GO: 0005496 |
| carbohydrate binding | GO: 0030246 |
| ion binding | GO: 0043167 |
| heme binding | GO: 0020037 |
| vitamin D binding | GO: 0005499 |
| hemoglobin binding | GO: 0030492 |

P-value < 0.05. The GO terms (biological process and molecular functions) shown are between GO levels 3 and 5.

From the 14 biomarker candidates, 10 PGCs were identified using a more stringent criterion (p-value <0.03) and constituted the proteomic biomarker panel (Table 8). Six of the biomarker panel PGCs (CFHR2, CPN1, APOB, HBB, GC, C9) were increased in CR relative to S, and four (IGFBP3, MST1, CDH5, C1QB) were decreased.

TABLE 8
Proteomic chronic cardiac allograft rejection biomarker panel.

| | | | | | | | SEQ |
|---|---|---|---|---|---|---|---|
| | | Gene | | | Fold | CR | ID |
| PGC | Accession # | Symbol | Protein Name | P.Value | Change | vs S | NO |
| 152 | IPI00556155.2 | IGFBP3 | insulin-like growth factor binding | 0.0006 | 1.30 | down | 11 |
| | | | protein 3 isoform a precursor | | | | |
| | IPI00855835.1 | — | Insulin-like growth factor binding | | | | 12 |
| | | | protein 3 isoform b | | | | |
| | IPI00018305.4 | IGFBP3 | Insulin-like growth factor-binding | | | | 14 |
| | | | protein 3 precursor | | | | |
| 126 | IPI00292218.4 | MST1 | Hepatocyte growth factor-like protein | 0.0036 | 1.45 | down | 15 |
| | | | precursor | | | | |
| | IPI00384647.1 | MST1 | Hepatocyte growth factor-like protein | | | | 36 |
| | | | homolog | | | | |
| | IPI00873854.1 | MSTP9 | 64 kDa protein | | | | 16 |
| 75 | IPI00218949.1 | CFHR2 | Isoform Short of Complement factor | 0.0044 | 1.27 | up | 17 |
| | | | H-related protein 2 precursor | | | | |
| | IPI00006154.1 | CFHR2 | Isoform Long of Complement factor | | | | 21 |
| | | | H-related protein 2 precursor | | | | |
| 78 | IPI00010295.1 | CPN1 | Carboxypeptidase N catalytic chain | 0.0097 | 1.21 | up | 22 |
| | | | precursor | | | | |
| 162 | IPI00012792.1 | CDH5 | Cadherin-5 precursor | 0.0114 | 1.32 | down | 23 |
| 270 | IPI00022229.1 | APOB | Apolipoprotein B-100 precursor | 0.0118 | 1.23 | up | 25 |
| 117 | IPI00473011.3 | HBB; HBD | Hemoglobin subunit delta | 0.0154 | 1.85 | up | 27 |
| | IPI00654755.3 | HBB | Hemoglobin subunit beta | | | | 28 |
| 96 | IPI00643948.2 | C1QB | Complement component 1, q | 0.0195 | 1.15 | down | 31 |
| | | | subcomponent, B chain | | | | |
| | IPI00477992.1 | C1QB | complement component 1, q | | | | 32 |
| | | | subcomponent, B chain precursor | | | | |
| 21 | IPI00555812.4 | GC | Vitamin D-binding protein precursor | 0.0222 | 2.27 | up | 33 |
| | IPI00742696.2 | GC | vitamin D-binding protein precursor | | | | 34 |
| 24 | IPI00022395.1 | C9 | Complement component C9 | 0.0241 | 1.24 | up | 35 |
| | | | precursor | | | | |

Similarly to the genomic analysis, an internal validation was performed to estimate the ability of the proteomic biomarker panel to classify 12 new samples (6 CR and 6 S). These samples were taken from the same patients at the same timepoint as those in the genomic internal validation. Using the classifier (developed based on the biomarker panel), one CR and one S sample were misclassified, resulting in a sensitivity and specificity of 83% for the proteomic chronic cardiac allograft rejection biomarker panel.

### Example 4

### Combinatorial Analysis Results

Results of the genomic and proteomic internal validations showed the same performance for both panels to distinguish between CR and S samples. As such, we examined the utility of a ‘combinatorial’ biomarker panel composed of both probe sets/genes and proteins. Four probe sets/genes (CLEC2B, CHPT1, 242907_at, GBP3) and four PGCs (CFHR2, CPN1, C1QB, GC) were separately identified using Step Discriminant Analysis (SDA) (Table 9).

TABLE 9
Combinatorial chronic cardiac allograft rejection biomarker panel.

| Affy ID/PGC- | | | | | Fold | CR |
|---|---|---|---|---|---|---|
| Accession# | | GeneSymbol | GeneTitle/Protein Name | P.Value | Change | vs S |
| 1556209_at | | CLEC2B | C-type lectin domain family 2, | 2.87E−05 | 2.39 | up |
| | | | member B | | | |
| 230364_at | | CHPT1 | choline phosphotransferase 1 | 1.14E−06 | 3.99 | up |
| 242907_at | | — | — | 2.64E−05 | 2.23 | up |
| 223434_at | | GBP3 | guanylate binding protein 3 | 3.69E−06 | 3.44 | up |
| 75 | IPI00218949.1 | CFHR2 | Isoform Short of Complement | 4.41E−03 | 1.27 | up |
| | | | factor H-related protein 2 | | | |
| | | | precursor | | | |
| | IPI00006154.1 | CFHR2 | Isoform Long of Complement | | | |
| | | | factor H-related protein 2 | | | |
| | | | precursor | | | |
| 78 | IPI00010295.1 | CPN1 | Carboxypeptidase N catalytic | 9.66E−03 | 1.21 | up |
| | | | chain precursor | | | |
| 96 | IPI00643948.2 | C1QB | Complement component 1, q | 1.95E−02 | 1.15 | down |
| | | | subcomponent, B chain | | | |
| | IPI00477992.1 | C1QB | complement component 1, q | | | |
| | | | subcomponent, B chain | | | |
| | | | precursor | | | |
| 21 | IPI00555812.4 | GC | Vitamin D-binding protein | 2.22E−02 | 2.27 | up |
| | | | precursor | | | |
| | IPI00742696.2 | GC | vitamin D-binding protein | | | |
| | | | precursor | | | |

The combinatorial panel was also evaluated using the same test cohort as described in the previous sections. The performance of the combinatorial panel was superior to that of either the genomic or the proteomic panels. The classifier built based on the combinatorial panel misclassified only one of the S samples, resulting in 100% sensitivity and 83% specificity (as compared to 83% sensitivity and specificity for the genomic and proteomic classifiers).

A striplot was constructed as a visualization tool to help summarize and compare the internal validation results for the genomic, proteomic, and combinatorial chronic cardiac allograft rejection biomarker panels (FIG. 6). To simplify the visualization, values for the linear discriminant (LD) variables for all three classifiers have been re-centered to calibrate the classification cut-off lines to zero. ‘HP4’, ‘H4’ and ‘Combinatorial’ represents the genomic, proteomic and combinatorial classifiers, respectively. Centers of the LD variable values (or the classifier ‘score’) for CR and S samples in the training set are shown using open and solid stars, respectively. The solid circles and solid squares correspond to the LD variable/classifier score for each of the S and CR samples, respectively in the test cohort. Samples with positive LD variables are classified as CR. The distance between the solid and open stars (average LD variable for the CR and S samples in the training cohort, respectively) illustrates the ability of the panels to jointly discriminate CR from S. The performance of each panel in jointly classifying new samples is illustrated with the solid circles and solid squares.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A method of determining the chronic allograft rejection status of a subject, the method comprising the steps of:
a. determining a genomic expression profile of one or more than one genomic markers in a biological sample from the subject, the genomic markers selected from the group comprising CHPT1, RPS26, GBP3, KLRC1/KLRC2, ZCCHC2, 242907_at, CLEC2B, PDK4, OSBP2 and IFIT5;
b. comparing the expression profile of the one or more than one genomic markers to a control profile; and
c. determining whether the expression level of the one or more than one genomic markers is increased or decreased relative to the control profile;
wherein the increase or decrease of the one or more than one genomic markers is indicative of the chronic rejection status of the subject.

2. The method of claim 1 wherein OSBP2 is increased relative to the non-rejector profile, and CHPT1, RPS26, GBP3, KLRC1/KLRC2, ZCCHC2, 242907_at, CLEC2B, PDK4 and IFIT5 are decreased relative to the control profile.

3. The method of claim 1 wherein the control profile is obtained from a non-rejecting, allograft recipient subject or a non-allograft recipient subject.

4. The method of claim 1, further comprising obtaining a value for one or more clinical variables.

5. The method of claim 1, further comprising at step a) determining the expression of one or more markers selected from Table 6.

6. The method of claim 1, wherein the expression profile of the one or more than one genomic markers is determined by detecting an RNA sequence corresponding to the one or more than one markers.

7. The method of claim 1, wherein the genomic expression profile of the one or more than one genomic markers is determined by PCR

8. The method of claim 1, wherein the genomic expression profile of the one or more than one genomic markers is determined by hybridization.

9. The method of claim 9, wherein the hybridization is to an oligonucleotide.

10. A method of determining the chronic allograft rejection status of a subject, the method comprising the steps of:
a. determining proteomic expression profile of one or more than one proteomic markers in a biological sample from the subject, the proteomic markers selected from the group comprising a polypeptide encoded by IGFBP3, MST1, CDH5, C1QB, CFHR2, CPN1, APOB, HBB, GC and C9;
b. comparing the expression profile of the one or more than one proteomic markers to a control profile; and
c. determining whether an expression level of the one or more than one proteomics markers is increased or decreased relative to the control profile;
wherein increase or decrease of the level of the one or more than one proteomic markers is indicative of the chronic rejection status of the subject.

11. The method of claim 10 wherein the level of polypeptides encoded by CFHR2, CPN1, APOB, HBB, GC and C9 are increased relative to a control profile, and the level of polypeptides encoded by IGFBP3, MST1, CDH5 and C1QB are increased relative to a control profile.

12. The method of claim 10 wherein the control profile is obtained from a non rejecting, allograft recipient subject or a non-allograft recipient subject.

13. The method of claim 10 further comprising obtaining a value for one or more clinical variables.

14. The method of claim 10, wherein the proteomic expression profile is determined by an immunologic assay.

15. The method of claim 10, wherein the proteomic expression profile is determined by ELISA.

16. The method of claim 10, wherein the proteomic expression profile is determined by mass spectrometry.

17. The method of claim 10, wherein the proteomic expression profile is determined by an isotope or isobaric tagging method.

18. The method of claim 1 wherein the control is an autologous control.

19. The method of claim 10 wherein the control is an autologous control.

20. A method of determining the chronic allograft rejection status of a subject using a combined panel of genomic and proteomic markers, the method comprising:
a) determining the genomic expression profile of CHPT1, GBP3, 242907_at and CLEC2B genomic markers in a biological sample from the subject;
b) determining proteomic expression profile of proteomic markers selected from the group comprising a polypeptide encoded by CFHR2, CPN1, GC and C1QB in the biological sample;
c) comparing the genomic and proteomic expression profile to a control profile; and
d) determining whether the genomic or proteomic expression level of the genomic and proteomic markers is increased or decreased relative to the control profile, wherein an increase in genomic markers CLDC2B, CHPT1, 242907_at, GB3 and an increase in the polypeptides encoded by CFHR2, CPN1 and GC and a decrease in the polypeptide encoded by C1QB is indicative of the chronic rejection status of the subject.
